SEQUENCE LISTING (353 sequences)

SEQ ID NO: 1 - DNA, 2043 bp, synthetic construct
  misc_feature 1..2043: note = EcoRI-HindIII fragment
  source 1..2043: mol_type = other DNA
gagctcgctcccccgccgtcgttcaatgagaatggataagaggctcgtgggattgacgtgagggggcagggatggctatatttctgggagaattaaccgatcgacgtgcaagcggacatttattttaaattcgataatttttgcaaaaacatttcgacatatttatttattttattattatgagaatcaatcctactacttctggttctggggtttccacggctactagcgaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtgggcaaagaacaaaaactcatttctgaagaagacttgtaactgcagataacccaaataatgttttaaaattttaaaaataatgtaggaggaaaaattatggctagcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagctctagctagagcgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgggggatcaaagctt

SEQ ID NO: 2 - DNA, 22 bp, synthetic construct
  misc_feature 1..22: note = primer
  source 1..22: mol_type = other DNA
ttttctgtcagtggagagggtg

SEQ ID NO: 3 - DNA, 20 bp, synthetic construct
  misc_feature 1..20: note = primer
  source 1..20: mol_type = other DNA
cccagcagctgttacaaact

SEQ ID NO: 4 - skipped

SEQ ID NO: 5 - protein, 100 aa, Arabidopsis thaliana
MEMRALGSSCSTGNGGSAPITLTNISPWITTVFPSTVKLRSSLRTFKGVSSRVRTFKGVSSTRVLSRTKQQFPLFCFLNPDPISFLENDVSEAERTVVLP

SEQ ID NO: 6 - DNA, 300 bp, Arabidopsis thaliana
  source 1..300: mol_type = genomic DNA
atggagatgagagctttgggttcttcgtgttctactggtaatggaggttctgctccgattaccctcacgaatatatctccatggatcacaacagtttttccgtcgacagtgaagctgagaagtagtttgagaaccttcaaaggagtttcgtcaagagtgagaacctttaaaggagtttcttcgacaagagttttgtctcggaccaaacaacagtttcctctgttttgtttcctaaaccctgatccgatctccttcttggaaaatgatgtatctgaagctgaaaggacagtagttttaccg

SEQ ID NO: 7 - protein, 100 aa, Arabidopsis lyrata
MEMRALVSSCATGNGGSDPFSFTKVSPWITTVGGKDRDFPTTVKLRTSMRTFKGVSIRGRTFKGVSTRVLSRNKQQFPLFCFLNPDPTSFRDNDISEAQR

SEQ ID NO: 8 - DNA, 300 bp, Arabidopsis lyrata
  source 1..300: mol_type = genomic DNA
atggagatgagagctttggtttcttcgtgtgctaccggtaatggaggttctgatccgtttagcttcacgaaagtttctccatggatcacaacagttggtggtaaggacagagattttccaacgacagtgaagctaagaactagtatgagaacctttaaaggagtttctataagagggagaacctttaaaggagtttcgacaagagttttgtctcggaacaaacaacagtttcctctgttttgtttcctaaaccctgatccgacctccttccgggataatgatatatctgaagctcaaagg

SEQ ID NO: 9 - DNA, 23 bp, synthetic construct
  misc_feature 1..23: note = gRNA
  source 1..23: mol_type = other DNA
ttggtttcttcgtgtgctaccgg

SEQ ID NO: 10 - DNA, 23 bp, synthetic construct
  misc_feature 1..23: note = gRNA
  source 1..23: mol_type = other DNA
tcttcgtgtgctaccggtaatgg

SEQ ID NO: 11 - DNA, 23 bp, synthetic construct
  misc_feature 1..23: note = gRNA
  source 1..23: mol_type = other DNA
tcgtgtgctaccggtaatggagg

SEQ ID NO: 12 - DNA, 23 bp, synthetic construct
  misc_feature 1..23: note = gRNA
  source 1..23: mol_type = other DNA
gcttcacgaaagtttctccatgg

SEQ ID NO: 13 - DNA, 23 bp, synthetic construct
  misc_feature 1..23: note = gRNA
  source 1..23: mol_type = other DNA
tctccatggatcacaacagttgg

SEQ ID NO: 14 - DNA, 23 bp, synthetic construct
  misc_feature 1..23: note = gRNA
  source 1..23: mol_type = other DNA
ccatggatcacaacagttggtgg

SEQ ID NO: 15 - DNA, 23 bp, synthetic construct
  misc_feature 1..23: note = gRNA
  source 1..23: mol_type = other DNA
gatcacaacagttggtggtaagg

SEQ ID NO: 16 - DNA, 23 bp, synthetic construct
  misc_feature 1..23: note = gRNA
  source 1..23: mol_type = other DNA
actagtatgagaacctttaaagg

SEQ ID NO: 17 - DNA, 23 bp, synthetic construct
  misc_feature 1..23: note = gRNA
  source 1..23: mol_type = other DNA
tttaaaggagtttctataagagg

SEQ ID NO: 18 - DNA, 23 bp, synthetic construct
  misc_feature 1..23: note = gRNA
  source 1..23: mol_type = other DNA
ataagagggagaacctttaaagg

SEQ ID NO: 19 - DNA, 23 bp, synthetic construct
  misc_feature 1..23: note = gRNA
  source 1..23: mol_type = other DNA
tttcgacaagagttttgtctcgg

SEQ ID NO: 20 - DNA, 23 bp, synthetic construct
  misc_feature 1..23: note = gRNA
  source 1..23: mol_type = other DNA
accctgatccgacctccttccgg

SEQ ID NO: 21 - DNA, 23 bp, synthetic construct
  misc_feature 1..23: note = gRNA
  source 1..23: mol_type = other DNA
atgatatatctgaagctcaaagg

SEQ ID NO: 22 - DNA, 300 bp, Arabidopsis lyrata
  source 1..300: mol_type = genomic DNA
cctttgagcttcagatatatcattatcccggaaggaggtcggatcagggtttaggaaacaaaacagaggaaactgttgtttgttccgagacaaaactcttgtcgaaactcctttaaaggttctccctcttatagaaactcctttaaaggttctcatactagttcttagcttcactgtcgttggaaaatctctgtccttaccaccaactgttgtgatccatggagaaactttcgtgaagctaaacggatcagaacctccattaccggtagcacacgaagaaaccaaagctctcatctccat

SEQ ID NO: 23 - DNA, 23 bp, synthetic construct
  misc_feature 1..23: note = gRNA
  source 1..23: mol_type = other DNA
cttcagatatatcattatcccgg

SEQ ID NO: 24 - DNA, 23 bp, synthetic construct
  misc_feature 1..23: note = gRNA
  source 1..23: mol_type = other DNA
agatatatcattatcccggaagg

SEQ ID NO: 25 - DNA, 23 bp, synthetic construct
  misc_feature 1..23: note = gRNA
  source 1..23: mol_type = other DNA
tatatcattatcccggaaggagg

SEQ ID NO: 26 - DNA, 23 bp, synthetic construct
  misc_feature 1..23: note = gRNA
  source 1..23: mol_type = other DNA
tcattatcccggaaggaggtcgg

SEQ ID NO: 27 - DNA, 23 bp, synthetic construct
  misc_feature 1..23: note = gRNA
  source 1..23: mol_type = other DNA
tcccggaaggaggtcggatcagg

SEQ ID NO: 28 - DNA, 23 bp, synthetic construct
  misc_feature 1..23: note = gRNA
  source 1..23: mol_type = other DNA
cccggaaggaggtcggatcaggg

SEQ ID NO: 29 - DNA, 23 bp, synthetic construct
  misc_feature 1..23: note = gRNA
  source 1..23: mol_type = other DNA
aggaggtcggatcagggtttagg

SEQ ID NO: 30 - DNA, 23 bp, synthetic construct
  misc_feature 1..23: note = gRNA
  source 1..23: mol_type = other DNA
gggtttaggaaacaaaacagagg

SEQ ID NO: 31 - DNA, 23 bp, synthetic construct
  misc_feature 1..23: note = gRNA
  source 1..23: mol_type = other DNA
tcttgtcgaaactcctttaaagg

SEQ ID NO: 32 - DNA, 23 bp, synthetic construct
  misc_feature 1..23: note = gRNA
  source 1..23: mol_type = other DNA
tcttatagaaactcctttaaagg

SEQ ID NO: 33 - DNA, 23 bp, synthetic construct
  misc_feature 1..23: note = gRNA
  source 1..23: mol_type = other DNA
gttcttagcttcactgtcgttgg

SEQ ID NO: 34 - DNA, 23 bp, synthetic construct
  misc_feature 1..23: note = gRNA
  source 1..23: mol_type = other DNA
ccaccaactgttgtgatccatgg

SEQ ID NO: 35 - DNA, 23 bp, synthetic construct
  misc_feature 1..23: note = gRNA
  source 1..23: mol_type = other DNA
gaaactttcgtgaagctaaacgg

SEQ ID NO: 36 - DNA, 23 bp, synthetic construct
  misc_feature 1..23: note = gRNA
  source 1..23: mol_type = other DNA
cggatcagaacctccattaccgg

SEQ ID NO: 37 - protein, 100 aa, Cannabis sativa
MEMRALVSSYSTGNGGSDPISLTNGSPWITTVGGGASTMDREFPLTVKLGSSMRAFKGVSTTTVLSRTKQQFPLVCLARNNANSTDPTSFWENDISEVQR

SEQ ID NO: 38 - DNA, 300 bp, Cannabis sativa
  source 1..300: mol_type = genomic DNA
atggagatgagagctttggtttcttcgtattctaccggtaatggaggttctgatccgatcagcctcacgaatggttctccatggatcacaacagttggtggtggtgcaagtaccatggacagagagtttccattgactgtgaagctgggaagtagtatgagagccttcaaaggagtaagcacaacaacagttttgtctcggaccaaacaacagtttcctctggtatgcttagcaagaaacaatgcgaacagcactgatccgacctcgttctgggagaatgatatatctgaagttcaaagg

SEQ ID NO: 39 - DNA, 23 bp, synthetic construct
  misc_feature 1..23: note = gRNA
  source 1..23: mol_type = other DNA
ttggtttcttcgtattctaccgg

SEQ ID NO: 40 - DNA, 23 bp, synthetic construct
  misc_feature 1..23: note = gRNA
  source 1..23: mol_type = other DNA
tcttcgtattctaccggtaatgg

SEQ ID NO: 41 - DNA, 23 bp, synthetic construct
  misc_feature 1..23: note = gRNA
  source 1..23: mol_type = other DNA
tcgtattctaccggtaatggagg

SEQ ID NO: 42 - DNA, 23 bp, synthetic construct
  misc_feature 1..23: note = gRNA
  source 1..23: mol_type = other DNA
gatccgatcagcctcacgaatgg

SEQ ID NO: 43 - DNA, 23 bp, synthetic construct
  misc_feature 1..23: note = gRNA
  source 1..23: mol_type = other DNA
gcctcacgaatggttctccatgg

SEQ ID NO: 44 - DNA, 23 bp, synthetic construct
  misc_feature 1..23: note = gRNA
  source 1..23: mol_type = other DNA
tctccatggatcacaacagttgg

SEQ ID NO: 45 - DNA, 23 bp, synthetic construct
  misc_feature 1..23: note = gRNA
  source 1..23: mol_type = other DNA
ccatggatcacaacagttggtgg

SEQ ID NO: 46 - DNA, 23 bp, synthetic construct
  misc_feature 1..23: note = gRNA
  source 1..23: mol_type = other DNA
tggatcacaacagttggtggtgg

SEQ ID NO: 47 - DNA, 23 bp, synthetic construct
  misc_feature 1..23: note = gRNA
  source 1..23: mol_type = other DNA
tggtggtggtgcaagtaccatgg

SEQ ID NO: 48 - DNA, 23 bp, synthetic construct
  misc_feature 1..23: note = gRNA
  source 1..23: mol_type = other DNA
gtttccattgactgtgaagctgg

SEQ ID NO: 49 - DNA, 23 bp, synthetic construct
  misc_feature 1..23: note = gRNA
  source 1..23: mol_type = other DNA
agtagtatgagagccttcaaagg

SEQ ID NO: 50 - DNA, 23 bp, synthetic construct
  misc_feature 1..23: note = gRNA
  source 1..23: mol_type = other DNA
gcacaacaacagttttgtctcgg

SEQ ID NO: 51 - DNA, 23 bp, synthetic construct
  misc_feature 1..23: note = gRNA
  source 1..23: mol_type = other DNA
gaccaaacaacagtttcctctgg

SEQ ID NO: 52 - DNA, 23 bp, synthetic construct
  misc_feature 1..23: note = gRNA
  source 1..23: mol_type = other DNA
gcactgatccgacctcgttctgg

SEQ ID NO: 53 - DNA, 23 bp, synthetic construct
  misc_feature 1..23: note = gRNA
  source 1..23: mol_type = other DNA
atgatatatctgaagttcaaagg

SEQ ID NO: 54 - DNA, 300 bp, Cannabis sativa
  source 1..300: mol_type = genomic DNA
cctttgaacttcagatatatcattctcccagaacgaggtcggatcagtgctgttcgcattgtttcttgctaagcataccagaggaaactgttgtttggtccgagacaaaactgttgttgtgcttactcctttgaaggctctcatactacttcccagcttcacagtcaatggaaactctctgtccatggtacttgcaccaccaccaactgttgtgatccatggagaaccattcgtgaggctgatcggatcagaacctccattaccggtagaatacgaagaaaccaaagctctcatctccat

SEQ ID NO: 55 - DNA, 23 bp, synthetic construct
  misc_feature 1..23: note = gRNA
  source 1..23: mol_type = other DNA
tatatcattctcccagaacgagg

SEQ ID NO: 56 - DNA, 23 bp, synthetic construct
  misc_feature 1..23: note = gRNA
  source 1..23: mol_type = other DNA
tcattctcccagaacgaggtcgg

SEQ ID NO: 57 - DNA, 23 bp, synthetic construct
  misc_feature 1..23: note = gRNA
  source 1..23: mol_type = other DNA
tttcttgctaagcataccagagg

SEQ ID NO: 58 - DNA, 23 bp, synthetic construct
  misc_feature 1..23: note = gRNA
  source 1..23: mol_type = other DNA
taccagaggaaactgttgtttgg

SEQ ID NO: 59 - DNA, 23 bp, synthetic construct
  misc_feature 1..23: note = gRNA
  source 1..23: mol_type = other DNA
tgttgtgcttactcctttgaagg

SEQ ID NO: 60 - DNA, 23 bp, synthetic construct
  misc_feature 1..23: note = gRNA
  source 1..23: mol_type = other DNA
cttcccagcttcacagtcaatgg

SEQ ID NO: 61 - DNA, 23 bp, synthetic construct
  misc_feature 1..23: note = gRNA
  source 1..23: mol_type = other DNA
caatggaaactctctgtccatgg

SEQ ID NO: 62 - DNA, 23 bp, synthetic construct
  misc_feature 1..23: note = gRNA
  source 1..23: mol_type = other DNA
ccaccaactgttgtgatccatgg

SEQ ID NO: 63 - DNA, 23 bp, synthetic construct
  misc_feature 1..23: note = gRNA
  source 1..23: mol_type = other DNA
tccatggagaaccattcgtgagg

SEQ ID NO: 64 - DNA, 23 bp, synthetic construct
  misc_feature 1..23: note = gRNA
  source 1..23: mol_type = other DNA
gaaccattcgtgaggctgatcgg

SEQ ID NO: 65 - DNA, 23 bp, synthetic construct
  misc_feature 1..23: note = gRNA
  source 1..23: mol_type = other DNA
cggatcagaacctccattaccgg

SEQ ID NO: 66 - protein, 100 aa, Cannabis sativa
MEMRALVSSCSTGNGGSDPISLTNGSPWITTVGGGASTMDREFPATVKLGSSMRAFKGVSTITVLSRTKQQFPLVCLARNNGNSTDPTSFWENDISETQR

SEQ ID NO: 67 - DNA, 300 bp, Cannabis sativa
  source 1..300: mol_type = genomic DNA
atggagatgagagctttggtttcttcgtgttctacggggaatggagggtctgatccgatcagcctcacgaatggttctccatggatcacaacagttggtggtggtgcaagtaccatggacagagagtttccagcgactgtgaagctgggaagtagtatgagagccttcaaaggagtaagcacaataacagttctgtctcggaccaaacaacagtttcctctggtatgcttagcaagaaacaacggaaacagcactgatccgacctcgttctgggagaacgatatatctgaaactcaaagg

SEQ ID NO: 68 - DNA, 23 bp, synthetic construct
  misc_feature 1..23: note = gRNA
  source 1..23: mol_type = other DNA
tttggtttcttcgtgttctacgg

SEQ ID NO: 69 - DNA, 23 bp, synthetic construct
  misc_feature 1..23: note = gRNA
  source 1..23: mol_type = other DNA
ttggtttcttcgtgttctacggg

SEQ ID NO: 70 - DNA, 23 bp, synthetic construct
  misc_feature 1..23: note = gRNA
  source 1..23: mol_type = other DNA
tggtttcttcgtgttctacgggg

SEQ ID NO: 71 - DNA, 23 bp, synthetic construct
  misc_feature 1..23: note = gRNA
  source 1..23: mol_type = other DNA
tcttcgtgttctacggggaatgg

SEQ ID NO: 72 - DNA, 23 bp, synthetic construct
  misc_feature 1..23: note = gRNA
  source 1..23: mol_type = other DNA
tcgtgttctacggggaatggagg

SEQ ID NO: 73 - DNA, 23 bp, synthetic construct
  misc_feature 1..23: note = gRNA
  source 1..23: mol_type = other DNA
gatccgatcagcctcacgaatgg

SEQ ID NO: 74 - DNA, 23 bp, synthetic construct
  misc_feature 1..23: note = gRNA
  source 1..23: mol_type = other DNA
gcctcacgaatggttctccatgg

SEQ ID NO: 75 - DNA, 23 bp, synthetic construct
  misc_feature 1..23: note = gRNA
  source 1..23: mol_type = other DNA
tctccatggatcacaacagttgg

SEQ ID NO: 76 - DNA, 23 bp, synthetic construct
  misc_feature 1..23: note = gRNA
  source 1..23: mol_type = other DNA
ccatggatcacaacagttggtgg

SEQ ID NO: 77 - DNA, 23 bp, synthetic construct
  misc_feature 1..23: note = gRNA
  source 1..23: mol_type = other DNA
tggatcacaacagttggtggtgg

SEQ ID NO: 78 - DNA, 23 bp, synthetic construct
  misc_feature 1..23: note = gRNA
  source 1..23: mol_type = other DNA
tggtggtggtgcaagtaccatgg

SEQ ID NO: 79 - DNA, 23 bp, synthetic construct
  misc_feature 1..23: note = gRNA
  source 1..23: mol_type = other DNA
gtttccagcgactgtgaagctgg

SEQ ID NO: 80 - DNA, 23 bp, synthetic construct
  misc_feature 1..23: note = gRNA
  source 1..23: mol_type = other DNA
agtagtatgagagccttcaaagg

SEQ ID NO: 81 - DNA, 23 bp, synthetic construct
  misc_feature 1..23: note = gRNA
  source 1..23: mol_type = other DNA
gcacaataacagttctgtctcgg

SEQ ID NO: 82 - DNA, 23 bp, synthetic construct
  misc_feature 1..23: note = gRNA
  source 1..23: mol_type = other DNA
gaccaaacaacagtttcctctgg

SEQ ID NO: 83 - DNA, 23 bp, synthetic construct
  misc_feature 1..23: note = gRNA
  source 1..23: mol_type = other DNA
gtatgcttagcaagaaacaacgg

SEQ ID NO: 84 - DNA, 23 bp, synthetic construct
  misc_feature 1..23: note = gRNA
  source 1..23: mol_type = other DNA
gcactgatccgacctcgttctgg

SEQ ID NO: 85 - DNA, 23 bp, synthetic construct
  misc_feature 1..23: note = gRNA
  source 1..23: mol_type = other DNA
acgatatatctgaaactcaaagg

SEQ ID NO: 86 - DNA, 300 bp, Cannabis sativa
  source 1..300: mol_type = genomic DNA
cctttgagtttcagatatatcgttctcccagaacgaggtcggatcagtgctgtttccgttgtttcttgctaagcataccagaggaaactgttgtttggtccgagacagaactgttattgtgcttactcctttgaaggctctcatactacttcccagcttcacagtcgctggaaactctctgtccatggtacttgcaccaccaccaactgttgtgatccatggagaaccattcgtgaggctgatcggatcagaccctccattccccgtagaacacgaagaaaccaaagctctcatctccat

SEQ ID NO: 87 - DNA, 23 bp, synthetic construct
  misc_feature 1..23: note = gRNA
  source 1..23: mol_type = other DNA
tatatcgttctcccagaacgagg

SEQ ID NO: 88 - DNA, 23 bp, synthetic construct
  misc_feature 1..23: note = gRNA
  source 1..23: mol_type = other DNA
tcgttctcccagaacgaggtcgg

SEQ ID NO: 89 - DNA, 23 bp, synthetic construct
  misc_feature 1..23: note = gRNA
  source 1..23: mol_type = other DNA
tttcttgctaagcataccagagg

SEQ ID NO: 90 - DNA, 23 bp, synthetic construct
  misc_feature 1..23: note = gRNA
  source 1..23: mol_type = other DNA
taccagaggaaactgttgtttgg

SEQ ID NO: 91 - DNA, 23 bp, synthetic construct
  misc_feature 1..23: note = gRNA
  source 1..23: mol_type = other DNA
tattgtgcttactcctttgaagg

SEQ ID NO: 92 - DNA, 23 bp, synthetic construct
  misc_feature 1..23: note = gRNA
  source 1..23: mol_type = other DNA
cttcccagcttcacagtcgctgg

SEQ ID NO: 93 - DNA, 23 bp, synthetic construct
  misc_feature 1..23: note = gRNA
  source 1..23: mol_type = other DNA
cgctggaaactctctgtccatgg

SEQ ID NO: 94 - DNA, 23 bp, synthetic construct
  misc_feature 1..23: note = gRNA
  source 1..23: mol_type = other DNA
ccaccaactgttgtgatccatgg

SEQ ID NO: 95 - DNA, 23 bp, synthetic construct
  misc_feature 1..23: note = gRNA
  source 1..23: mol_type = other DNA
tccatggagaaccattcgtgagg

SEQ ID NO: 96 - DNA, 23 bp, synthetic construct
  misc_feature 1..23: note = gRNA
  source 1..23: mol_type = other DNA
gaaccattcgtgaggctgatcgg

SEQ ID NO: 97 - protein, 100 aa, Capsella sp.
MEMRALVSSCSTGNGGSDPISLTNVSPWITTVGGGASSIDREFPTTVKLGSSLRTFKGVSSTTVLSRTKQQFPLVCLARNNANSTDPTLFWENDISEAQS

SEQ ID NO: 98 - DNA, 300 bp, Capsella sp.
  source 1..300: mol_type = genomic DNA
atggagatgagagctttggtttcttcgtgttctaccggtaatggaggttctgatccgattagcctcacgaatgtttctccatggatcacaacagttggtggtggtgcaagttccattgacagagagtttccaacgactgtgaagctgggaagtagtctgagaactttcaaaggagtaagctctacgacagttttgtctcggaccaaacaacagtttcctctggtttgtttagcaagaaacaatgccaacagcactgatccaaccttgttctgggaaaatgacatatctgaagctcaaagc

SEQ ID NO: 99 - DNA, 23 bp, synthetic construct
  misc_feature 1..23: note = gRNA
  source 1..23: mol_type = other DNA
ttggtttcttcgtgttctaccgg

SEQ ID NO: 100 - DNA, 23 bp, synthetic construct
  misc_feature 1..23: note = gRNA
  source 1..23: mol_type = other DNA
tcttcgtgttctaccggtaatgg

SEQ ID NO: 101 - DNA, 23 bp, synthetic construct
  misc_feature 1..23: note = gRNA
  source 1..23: mol_type = other DNA
tcgtgttctaccggtaatggagg

SEQ ID NO: 102 - DNA, 23 bp, synthetic construct
  misc_feature 1..23: note = gRNA
  source 1..23: mol_type = other DNA
gcctcacgaatgtttctccatgg

SEQ ID NO: 103 - DNA, 23 bp, synthetic construct
  misc_feature 1..23: note = gRNA
  source 1..23: mol_type = other DNA
tctccatggatcacaacagttgg

SEQ ID NO: 104 - DNA, 23 bp, synthetic construct
  misc_feature 1..23: note = gRNA
  source 1..23: mol_type = other DNA
ccatggatcacaacagttggtgg

SEQ ID NO: 105 - DNA, 23 bp, synthetic construct
  misc_feature 1..23: note = gRNA
  source 1..23: mol_type = other DNA
gtttccaacgactgtgaagctgg

SEQ ID NO: 106 - DNA, 23 bp, synthetic construct
  misc_feature 1..23: note = gRNA
  source 1..23: mol_type = other DNA
agtagtctgagaactttcaaagg

SEQ ID NO: 107 - DNA, 23 bp, synthetic construct
  misc_feature 1..23: note = gRNA
  source 1..23: mol_type = other DNA
gctctacgacagttttgtctcgg

SEQ ID NO: 108 - DNA, 23 bp, synthetic construct
  misc_feature 1..23: note = gRNA
  source 1..23: mol_type = other DNA
gaccaaacaacagtttcctctgg

SEQ ID NO: 109 - DNA, 23 bp, synthetic construct
  misc_feature 1..23: note = gRNA
  source 1..23: mol_type = other DNA
gcactgatccaaccttgttctgg

SEQ ID NO: 110 - DNA, 300 bp, Capsella sp.
  source 1..300: mol_type = genomic DNA
gctttgagcttcagatatgtcattttcccagaacaaggttggatcagtgctgttggcattgtttcttgctaaacaaaccagaggaaactgttgtttggtccgagacaaaactgtcgtagagcttactcctttgaaagttctcagactacttcccagcttcacagtcgttggaaactctctgtcaatggaacttgcaccaccaccaactgttgtgatccatggagaaacattcgtgaggctaatcggatcagaacctccattaccggtagaacacgaagaaaccaaagctctcatctccat

SEQ ID NO: 111 - DNA, 23 bp, synthetic construct
  misc_feature 1..23: note = gRNA
  source 1..23: mol_type = other DNA
tatgtcattttcccagaacaagg

SEQ ID NO: 112 - DNA, 23 bp, synthetic construct
  misc_feature 1..23: note = gRNA
  source 1..23: mol_type = other DNA
tcattttcccagaacaaggttgg

SEQ ID NO: 113 - DNA, 23 bp, synthetic construct
  misc_feature 1..23: note = gRNA
  source 1..23: mol_type = other DNA
caaggttggatcagtgctgttgg

SEQ ID NO: 114 - DNA, 23 bp, synthetic construct
  misc_feature 1..23: note = gRNA
  source 1..23: mol_type = other DNA
tttcttgctaaacaaaccagagg

SEQ ID NO: 115 - DNA, 23 bp, synthetic construct
  misc_feature 1..23: note = gRNA
  source 1..23: mol_type = other DNA
aaccagaggaaactgttgtttgg

SEQ ID NO: 116 - DNA, 23 bp, synthetic construct
  misc_feature 1..23: note = gRNA
  source 1..23: mol_type = other DNA
cttcccagcttcacagtcgttgg

SEQ ID NO: 117 - DNA, 23 bp, synthetic construct
  misc_feature 1..23: note = gRNA
  source 1..23: mol_type = other DNA
cgttggaaactctctgtcaatgg

SEQ ID NO: 118 - DNA, 23 bp, synthetic construct
  misc_feature 1..23: note = gRNA
  source 1..23: mol_type = other DNA
ccaccaactgttgtgatccatgg

SEQ ID NO: 119 - DNA, 23 bp, synthetic construct
  misc_feature 1..23: note = gRNA
  source 1..23: mol_type = other DNA
tccatggagaaacattcgtgagg

SEQ ID NO: 120 - DNA, 23 bp, synthetic construct
  misc_feature 1..23: note = gRNA
  source 1..23: mol_type = other DNA
gaaacattcgtgaggctaatcgg

SEQ ID NO: 121 - DNA, 23 bp, synthetic construct
  misc_feature 1..23: note = gRNA
  source 1..23: mol_type = other DNA
cggatcagaacctccattaccgg

SEQ ID NO: 122 - protein, 100 aa, Brassica oleracea
MEMRALVSCSAAGNGASDRFRLSNVSPWITSARGASGSDSPATVKLRSSSMIRAFKGVSIYKNKTRRNVLSQRNKQFRPMAYLGRKDLSSPDPTSFCDND

SEQ ID NO: 123 - DNA, 300 bp, Brassica oleracea
  source 1..300: mol_type = genomic DNA
atggagatgagagctttggtttcgtgttctgctgccggaaatggagcttctgatcggtttagactctccaatgtttcaccatggatcacatctgctcgtggtgcaagtggcagtgactccccagccacagtgaagctgagaagcagctctatgattagagctttcaaaggagtttcgatttacaaaaacaagaccagaagaaatgttctgtctcaaaggaacaaacagttccgtcctatggcctacttaggaaggaaggacttgagcagccctgatccgacctccttctgcgataatgat

SEQ ID NO: 124 - DNA, 23 bp, synthetic construct
  misc_feature 1..23: note = gRNA
  source 1..23: mol_type = other DNA
ttggtttcgtgttctgctgccgg

SEQ ID NO: 125 - DNA, 23 bp, synthetic construct
  misc_feature 1..23: note = gRNA
  source 1..23: mol_type = other DNA
tcgtgttctgctgccggaaatgg

SEQ ID NO: 126 - DNA, 23 bp, synthetic construct
  misc_feature 1..23: note = gRNA
  source 1..23: mol_type = other DNA
ccggaaatggagcttctgatcgg

SEQ ID NO: 127 - DNA, 23 bp, synthetic construct
  misc_feature 1..23: note = gRNA
  source 1..23: mol_type = other DNA
gactctccaatgtttcaccatgg

SEQ ID NO: 128 - DNA, 23 bp, synthetic construct
  misc_feature 1..23: note = gRNA
  source 1..23: mol_type = other DNA
ccatggatcacatctgctcgtgg

SEQ ID NO: 129 - DNA, 23 bp, synthetic construct
  misc_feature 1..23: note = gRNA
  source 1..23: mol_type = other DNA
acatctgctcgtggtgcaagtgg

SEQ ID NO: 130 - DNA, 23 bp, synthetic construct
  misc_feature 1..23: note = gRNA
  source 1..23: mol_type = other DNA
tctatgattagagctttcaaagg

SEQ ID NO: 131 - DNA, 23 bp, synthetic construct
  misc_feature 1..23: note = gRNA
  source 1..23: mol_type = other DNA
gaagaaatgttctgtctcaaagg

SEQ ID NO: 132 - DNA, 23 bp, synthetic construct
  misc_feature 1..23: note = gRNA
  source 1..23: mol_type = other DNA
gaacaaacagttccgtcctatgg

SEQ ID NO: 133 - DNA, 23 bp, synthetic construct
  misc_feature 1..23: note = gRNA
  source 1..23: mol_type = other DNA
ttccgtcctatggcctacttagg

SEQ ID NO: 134 - DNA, 23 bp, synthetic construct
  misc_feature 1..23: note = gRNA
  source 1..23: mol_type = other DNA
gtcctatggcctacttaggaagg

SEQ ID NO: 135 - DNA, 23 bp, synthetic construct
  misc_feature 1..23: note = gRNA
  source 1..23: mol_type = other DNA
tatggcctacttaggaaggaagg

SEQ ID NO: 136 - DNA, 300 bp, Brassica oleracea
  source 1..300: mol_type = genomic DNA
atcattatcgcagaaggaggtcggatcagggctgctcaagtccttccttcctaagtaggccataggacggaactgtttgttcctttgagacagaacatttcttctggtcttgtttttgtaaatcgaaactcctttgaaagctctaatcatagagctgcttctcagcttcactgtggctggggagtcactgccacttgcaccacgagcagatgtgatccatggtgaaacattggagagtctaaaccgatcagaagctccatttccggcagcagaacacgaaaccaaagctctcatctccat

SEQ ID NO: 137 - DNA, 23 bp, synthetic construct
  misc_feature 1..23: note = gRNA
  source 1..23: mol_type = other DNA
tcattatcgcagaaggaggtcgg

SEQ ID NO: 138 - DNA, 23 bp, synthetic construct
  misc_feature 1..23: note = gRNA
  source 1..23: mol_type = other DNA
tcgcagaaggaggtcggatcagg

SEQ ID NO: 139 - DNA, 23 bp, synthetic construct
  misc_feature 1..23: note = gRNA
  source 1..23: mol_type = other DNA
caagtccttccttcctaagtagg

SEQ ID NO: 140 - DNA, 23 bp, synthetic construct
  misc_feature 1..23: note = gRNA
  source 1..23: mol_type = other DNA
ttccttcctaagtaggccatagg

SEQ ID NO: 141 - DNA, 23 bp, synthetic construct
  misc_feature 1..23: note = gRNA
  source 1..23: mol_type = other DNA
ttcctaagtaggccataggacgg

SEQ ID NO: 142 - DNA, 23 bp, synthetic construct
  misc_feature 1..23: note = gRNA
  source 1..23: mol_type = other DNA
tggatcacaacagttggtggtgg

SEQ ID NO: 143 - DNA, 23 bp, synthetic construct
  misc_feature 1..23: note = gRNA
  source 1..23: mol_type = other DNA
ttgagacagaacatttcttctgg

SEQ ID NO: 144 - DNA, 23 bp, synthetic construct
  misc_feature 1..23: note = gRNA
  source 1..23: mol_type = other DNA
gctgcttctcagcttcactgtgg

SEQ ID NO: 145 - DNA, 23 bp, synthetic construct
  misc_feature 1..23: note = gRNA
  source 1..23: mol_type = other DNA
cttctcagcttcactgtggctgg

SEQ ID NO: 146 - DNA, 23 bp, synthetic construct
  misc_feature 1..23: note = gRNA
  source 1..23: mol_type = other DNA
ttctcagcttcactgtggctggg

SEQ ID NO: 147 - DNA, 23 bp, synthetic construct
  misc_feature 1..23: note = gRNA
  source 1..23: mol_type = other DNA
tctcagcttcactgtggctgggg

SEQ ID NO: 148 - DNA, 23 bp, synthetic construct
  misc_feature 1..23: note = gRNA
  source 1..23: mol_type = other DNA
ccacgagcagatgtgatccatgg

SEQ ID NO: 149 - DNA, 23 bp, synthetic construct
  misc_feature 1..23: note = gRNA
  source 1..23: mol_type = other DNA
tgtgatccatggtgaaacattgg

SEQ ID NO: 150 - DNA, 23 bp, synthetic construct
  misc_feature 1..23: note = gRNA
  source 1..23: mol_type = other DNA
ccgatcagaagctccatttccgg

SEQ ID NO: 151 - protein, 371 aa, Brassica napus
MEMRALVSCSAAGNGASDRFRLSNVSPWITSARGASGSDSPATVKLGSSSMIRAFKGVSIYKNKTRRNVLSQRNKQFRPMAYLGRKDLSSPDPTSFCDNDISEPQGTGSINGNDHSAVRVSQVDEFCKAHGGKRPIHSILVATNGMAAVKLIRSVRAWSYQTFGSEKSISLVAMATPEDMRINAEHIRIADQFMQVPGGTNNNNYANVHLIVEMAQATGVDAVWPGWGHASENPELPDALKAKGVIFLGPTAASMLALGDKIGSSLIAQAADVPTLPWSGSHVKIPPGSSMVTIPEEMYRQACVYTTEEAVASCQVVGYPAMIKASWGGGGKGIREVHDDDEVRTLFKQVQGEVPGSPIFIMKVASQSRHL

SEQ ID NO: 152 - DNA, 298 bp, Brassica napus
  source 1..298: mol_type = genomic DNA
atggagatgagagctttagtttcgtgttctgctgccggaaatggagcttctgatcggtttagactctccaatgtttcaccatggatcacatcagctcgtggtgcaagtggcagtgactccccagccacagtgaagctgggaagcagctctatgattagagctttcaaaggcgtttcgatttacaaaaacaagaccagaaggaatgttctgtctcaaaggaacaaacagttccgtcctatggcctacttaggaaggaaggacttgagcagccctgatccgacctccttctgcgataatg

SEQ ID NO: 153 - DNA, 23 bp, synthetic construct
  misc_feature 1..23: note = gRNA
  source 1..23: mol_type = other DNA
ttagtttcgtgttctgctgccgg

SEQ ID NO: 154 - DNA, 23 bp, synthetic construct
  misc_feature 1..23: note = gRNA
  source 1..23: mol_type = other DNA
tcgtgttctgctgccggaaatgg

SEQ ID NO: 155 - DNA, 23 bp, synthetic construct
  misc_feature 1..23: note = gRNA
  source 1..23: mol_type = other DNA
ccggaaatggagcttctgatcgg

SEQ ID NO: 156 - DNA, 23 bp, synthetic construct
  misc_feature 1..23: note = gRNA
  source 1..23: mol_type = other DNA
gactctccaatgtttcaccatgg

SEQ ID NO: 157 - DNA, 23 bp, synthetic construct
  misc_feature 1..23: note = gRNA
  source 1..23: mol_type = other DNA
ccatggatcacatcagctcgtgg

SEQ ID NO: 158 - DNA, 23 bp, synthetic construct
  misc_feature 1..23: note = gRNA
  source 1..23: mol_type = other DNA
acatcagctcgtggtgcaagtgg

SEQ ID NO: 159 - DNA, 23 bp, synthetic construct
  misc_feature 1..23: note = gRNA
  source 1..23: mol_type = other DNA
ctccccagccacagtgaagctgg

SEQ ID NO: 160 - DNA, 23 bp, synthetic construct
  misc_feature 1..23: note = gRNA
  source 1..23: mol_type = other DNA
tccccagccacagtgaagctggg

SEQ ID NO: 161 - DNA, 23 bp, synthetic construct
  misc_feature 1..23: note = gRNA
  source 1..23: mol_type = other DNA
tctatgattagagctttcaaagg

SEQ ID NO: 162 - DNA, 23 bp, synthetic construct
  misc_feature 1..23: note = gRNA
  source 1..23: mol_type = other DNA
tttacaaaaacaagaccagaagg

SEQ ID NO: 163 - DNA, 23 bp, synthetic construct
  misc_feature 1..23: note = gRNA
  source 1..23: mol_type = other DNA
gaaggaatgttctgtctcaaagg

SEQ ID NO: 164 - DNA, 23 bp, synthetic construct
  misc_feature 1..23: note = gRNA
  source 1..23: mol_type = other DNA
gaacaaacagttccgtcctatgg

SEQ ID NO: 165 - DNA, 23 bp, synthetic construct
  misc_feature 1..23: note = gRNA
  source 1..23: mol_type = other DNA
ttccgtcctatggcctacttagg

SEQ ID NO: 166 - DNA, 23 bp, synthetic construct
  misc_feature 1..23: note = gRNA
  source 1..23: mol_type = other DNA
gtcctatggcctacttaggaagg

SEQ ID NO: 167 - DNA, 23 bp, synthetic construct
  misc_feature 1..23: note = gRNA
  source 1..23: mol_type = other DNA
tatggcctacttaggaaggaagg

SEQ ID NO: 168 - DNA, 298 bp, Brassica napus
  source 1..298: mol_type = genomic DNA
cattatcgcagaaggaggtcggatcagggctgctcaagtccttccttcctaagtaggccataggacggaactgtttgttcctttgagacagaacattccttctggtcttgtttttgtaaatcgaaacgcctttgaaagctctaatcatagagctgcttcccagcttcactgtggctggggagtcactgccacttgcaccacgagctgatgtgatccatggtgaaacattggagagtctaaaccgatcagaagctccatttccggcagcagaacacgaaactaaagctctcatctccat

SEQ ID NO: 169 - DNA, 23 bp, synthetic construct
  misc_feature 1..23: note = gRNA
  source 1..23: mol_type = other DNA
tcgcagaaggaggtcggatcagg

SEQ ID NO: 170 - DNA, 23 bp, synthetic construct
  misc_feature 1..23: note = gRNA
  source 1..23: mol_type = other DNA
cgcagaaggaggtcggatcaggg

SEQ ID NO: 171 - DNA, 23 bp, synthetic construct
  misc_feature 1..23: note = gRNA
  source 1..23: mol_type = other DNA
caagtccttccttcctaagtagg

SEQ ID NO: 172 - DNA, 23 bp, synthetic construct
  misc_feature 1..23: note = gRNA
  source 1..23: mol_type = other DNA
ttccttcctaagtaggccatagg

SEQ ID NO: 173 - DNA, 23 bp, synthetic construct
  misc_feature 1..23: note = gRNA
  source 1..23: mol_type = other DNA
ttcctaagtaggccataggacgg

SEQ ID NO: 174 - DNA, 23 bp, synthetic construct
  misc_feature 1..23: note = gRNA
  source 1..23: mol_type = other DNA
ttgagacagaacattccttctgg

SEQ ID NO: 175 - DNA, 23 bp, synthetic construct
  misc_feature 1..23: note = gRNA
  source 1..23: mol_type = other DNA
cttcccagcttcactgtggctgg

SEQ ID NO: 176 - DNA, 23 bp, synthetic construct
  misc_feature 1..23: note = gRNA
  source 1..23: mol_type = other DNA
ttcccagcttcactgtggctggg

SEQ ID NO: 177 - DNA, 23 bp, synthetic construct
  misc_feature 1..23: note = gRNA
  source 1..23: mol_type = other DNA
tcccagcttcactgtggctgggg

SEQ ID NO: 178 - DNA, 23 bp, synthetic construct
  misc_feature 1..23: note = gRNA
  source 1..23: mol_type = other DNA
ccacgagctgatgtgatccatgg

SEQ ID NO: 179 - DNA, 23 bp, synthetic construct
  misc_feature 1..23: note = gRNA
  source 1..23: mol_type = other DNA
tgtgatccatggtgaaacattgg

SEQ ID NO: 180 - DNA, 23 bp, synthetic construct
  misc_feature 1..23: note = gRNA
  source 1..23: mol_type = other DNA
ccgatcagaagctccatttccgg

SEQ ID NO: 181 - protein, 455 aa, Brassica napus
MEMRALVSCSAAGNGASDRFRLSNVSPWITSARGASGSDSPATVKLGSSSMIRAFKGVSIYKNKTRRNVLSQRNKQFRPMAYLGRKDLSSPDPTSFCDNDISEPQGTGSINGNDHSAVRVSQVDEFCKAHGGKRPIHRILVATNGMAAVKFIRSVRAWSYQTFGSEKSISLVAMATPEDMRINAEHIRIADQFMQVPGGTNNNNYANVHLIVEMAEATGVDAVWPGWGHASENPELPDALKAKGVIFLGPTAASMLALGDKIGSSLIAQAADVPTLPWSGSHVKIPPGSSLVTIPEEMYRQACVYTTEEAVASCQVVGYPAMIKASWGGGGKGIRKVHDDDEVRALFKQVQGEVPGSPIFIMKVASQSRHLEVQLLCDQYGNVSALHSRDCSVQRRHQKIIEEGPITVAPRDTVKKLEQAARRLAKSVNYVGAATVEFLYSMDTGDYFFLELNPR

SEQ ID NO: 182 - DNA, 298 bp, Brassica napus
  source 1..298: mol_type = genomic DNA
atggagatgagagctttggtttcgtgttctgctgccggaaatggagcttctgatcggtttagactctccaatgtttcaccatggatcacatcagctcgtggtgcaagtggcagtgactccccagccacagtgaagctgggaagcagctctatgatcagagccttcaaaggagtttcgatttacaaaaacaagaccagaagaaatgttttgtctcaaaggaacaaacagtttcgtcctatggcctacttaggaaggaaggacttgagcagccctgatccgacctccttctgcgataatg

SEQ ID NO: 183 - DNA, 23 bp, synthetic construct
  misc_feature 1..23: note = gRNA
  source 1..23: mol_type = other DNA
ttggtttcgtgttctgctgccgg

SEQ ID NO: 184 - DNA, 23 bp, synthetic construct
  misc_feature 1..23: note = gRNA
  source 1..23: mol_type = other DNA
tcgtgttctgctgccggaaatgg

SEQ ID NO: 185 - DNA, 23 bp, synthetic construct
  misc_feature 1..23: note = gRNA
  source 1..23: mol_type = other DNA
ccggaaatggagcttctgatcgg

SEQ ID NO: 186 - DNA, 23 bp, synthetic construct
  misc_feature 1..23: note = gRNA
  source 1..23: mol_type = other DNA
gactctccaatgtttcaccatgg

SEQ ID NO: 187 - DNA, 23 bp, synthetic construct
  misc_feature 1..23: note = gRNA
  source 1..23: mol_type = other DNA
ccatggatcacatcagctcgtgg

SEQ ID NO: 188 - DNA, 23 bp, synthetic construct
  misc_feature 1..23: note = gRNA
  source 1..23: mol_type = other DNA
acatcagctcgtggtgcaagtgg

SEQ ID NO: 189 - DNA, 23 bp, synthetic construct
  misc_feature 1..23: note = gRNA
  source 1..23: mol_type = other DNA
ctccccagccacagtgaagctgg

SEQ ID NO: 190 - DNA, 23 bp, synthetic construct
  misc_feature 1..23: note = gRNA
  source 1..23: mol_type = other DNA
tccccagccacagtgaagctggg

SEQ ID NO: 191 - DNA, 23 bp, synthetic construct
  misc_feature 1..23: note = gRNA
  source 1..23: mol_type = other DNA
tctatgatcagagccttcaaagg

SEQ ID NO: 192 - DNA, 23 bp, synthetic construct
  misc_feature 1..23: note = gRNA
  source 1..23: mol_type = other DNA
gaagaaatgttttgtctcaaagg

SEQ ID NO: 193 - DNA, 23 bp, synthetic construct
  misc_feature 1..23: note = gRNA
  source 1..23: mol_type = other DNA
gaacaaacagtttcgtcctatgg

SEQ ID NO: 194 - DNA, 23 bp, synthetic construct
  misc_feature 1..23: note = gRNA
  source 1..23: mol_type = other DNA
tttcgtcctatggcctacttagg

SEQ ID NO: 195 - DNA, 23 bp, synthetic construct
  misc_feature 1..23: note = gRNA
  source 1..23: mol_type = other DNA
gtcctatggcctacttaggaagg

SEQ ID NO: 196 - DNA, 23 bp, synthetic construct
  misc_feature 1..23: note = gRNA
  source 1..23: mol_type = other DNA
tatggcctacttaggaaggaagg

SEQ ID NO: 197 - DNA, 298 bp, Brassica napus
  source 1..298: mol_type = genomic DNA
cattatcgcagaaggaggtcggatcagggctgctcaagtccttccttcctaagtaggccataggacgaaactgtttgttcctttgagacaaaacatttcttctggtcttgtttttgtaaatcgaaactcctttgaaggctctgatcatagagctgcttcccagcttcactgtggctggggagtcactgccacttgcaccacgagctgatgtgatccatggtgaaacattggagagtctaaaccgatcagaagctccatttccggcagcagaacacgaaaccaaagctctcatctccat

SEQ ID NO: 198 - DNA, 23 bp, synthetic construct
  misc_feature 1..23: note = gRNA
  source 1..23: mol_type = other DNA
tcgcagaaggaggtcggatcagg

SEQ ID NO: 199 - DNA, 23 bp, synthetic construct
  misc_feature 1..23: note = gRNA
  source 1..23: mol_type = other DNA
cgcagaaggaggtcggatcaggg

SEQ ID NO: 200 - DNA, 23 bp, synthetic construct
  misc_feature 1..23: note = gRNA
  source 1..23: mol_type = other DNA
caagtccttccttcctaagtagg

SEQ ID NO: 201 - DNA, 23 bp, synthetic construct
  misc_feature 1..23: note = gRNA
  source 1..23: mol_type = other DNA
ttccttcctaagtaggccatagg

SEQ ID NO: 202 - DNA, 23 bp, synthetic construct
  misc_feature 1..23: note = gRNA
  source 1..23: mol_type = other DNA
ttgagacaaaacatttcttctgg

SEQ ID NO: 203 - DNA, 23 bp, synthetic construct
  misc_feature 1..23: note = gRNA
  source 1..23: mol_type = other DNA
gtaaatcgaaactcctttgaagg

SEQ ID NO: 204 - DNA, 23 bp, synthetic construct
  misc_feature 1..23: note = gRNA
  source 1..23: mol_type = other DNA
gctgcttcccagcttcactgtgg

SEQ ID NO: 205 - DNA, 23 bp, synthetic construct
  misc_feature 1..23: note = gRNA
  source 1..23: mol_type = other DNA
cttcccagcttcactgtggctgg

SEQ ID NO: 206 - DNA, 23 bp, synthetic construct
  misc_feature 1..23: note = gRNA
  source 1..23: mol_type = other DNA
ttcccagcttcactgtggctggg

SEQ ID NO: 207 - DNA, 23 bp, synthetic construct
  misc_feature 1..23: note = gRNA
  source 1..23: mol_type = other DNA
tcccagcttcactgtggctgggg

SEQ ID NO: 208 - DNA, 23 bp, synthetic construct
  misc_feature 1..23: note = gRNA
  source 1..23: mol_type = other DNA
ccacgagctgatgtgatccatgg

SEQ ID NO: 209 - DNA, 23 bp, synthetic construct
  misc_feature 1..23: note = gRNA
  source 1..23: mol_type = other DNA
tgtgatccatggtgaaacattgg

SEQ ID NO: 210 - DNA, 23 bp, synthetic construct
  misc_feature 1..23: note = gRNA
  source 1..23: mol_type = other DNA
ccgatcagaagctccatttccgg

SEQ ID NO: 211 - protein, 100 aa, Brassica rapa
MEMRALVSCSAAGNGASDRFRLSNVSPWITSARGASGSDSPATVKLGSSSMIRAFKGVSIYKNKSRRNVLSQRNKQFRPMAYLGRKDLSSPDPTSFCDND

SEQ ID NO: 212 - DNA, 300 bp, Brassica rapa
  source 1..300: mol_type = genomic DNA
atggagatgagagctttggtttcgtgttctgctgccggaaatggagcttctgatcggtttagactctccaatgtttcaccatggatcacatcagctcgtggtgcaagtggcagtgactccccagccacagtgaagctgggaagcagctctatgatcagagccttcaaaggagtttcgatttacaaaaacaagagcagaagaaatgttctgtctcaaaggaacaaacagtttcgtcctatggcctacttaggaaggaaggacttgagcagccctgatccgacctccttctgcgataatgat

SEQ ID NO: 213 - DNA, 23 bp, synthetic construct
  misc_feature 1..23: note = gRNA
  source 1..23: mol_type = other DNA
ttggtttcgtgttctgctgccgg

SEQ ID NO: 214 - DNA, 23 bp, synthetic construct
  misc_feature 1..23: note = gRNA
  source 1..23: mol_type = other DNA
tcgtgttctgctgccggaaatgg

SEQ ID NO: 215 - DNA, 23 bp, synthetic construct
  misc_feature 1..23: note = gRNA
  source 1..23: mol_type = other DNA
ccggaaatggagcttctgatcgg

SEQ ID NO: 216 - DNA, 23 bp, synthetic construct
  misc_feature 1..23: note = gRNA
  source 1..23: mol_type = other DNA
gactctccaatgtttcaccatgg

SEQ ID NO: 217 - DNA, 23 bp, synthetic construct
  misc_feature 1..23: note = gRNA
  source 1..23: mol_type = other DNA
ccatggatcacatcagctcgtgg

SEQ ID NO: 218 - DNA, 23 bp, synthetic construct
  misc_feature 1..23: note = gRNA
  source 1..23: mol_type = other DNA
acatcagctcgtggtgcaagtgg

SEQ ID NO: 219 - DNA, 23 bp, synthetic construct
  misc_feature 1..23: note = gRNA
  source 1..23: mol_type = other DNA
ctccccagccacagtgaagctgg

SEQ ID NO: 220 - DNA, 23 bp, synthetic construct
  misc_feature 1..23: note = gRNA
  source 1..23: mol_type = other DNA
tctatgatcagagccttcaaagg

SEQ ID NO: 221 - DNA, 23 bp, synthetic construct
  misc_feature 1..23: note = gRNA
  source 1..23: mol_type = other DNA
gaagaaatgttctgtctcaaagg

SEQ ID NO: 222 - protein, 95 aa, Arabidopsis thaliana
MAGSVNGNHSAVGPGINYETVSQVDEFCKALRGKRPIHSILIANNGMAAVKFIRSVRTWAYETFGTEKAILLVGMATPEDMRINAEHIRIADQFV

SEQ ID NO: 223 - DNA, 23 bp, synthetic construct
  misc_feature 1..23: note = gRNA
  source 1..23: mol_type = other DNA
tttcgtcctatggcctacttagg

SEQ ID NO: 224 - DNA, 23 bp, synthetic construct
  misc_feature 1..23: note = gRNA
  source 1..23: mol_type = other DNA
gtcctatggcctacttaggaagg

SEQ ID NO: 225 - DNA, 23 bp, synthetic construct
  misc_feature 1..23: note = gRNA
  source 1..23: mol_type = other DNA
tatggcctacttaggaaggaagg

SEQ ID NO: 226 - DNA, 300 bp, Brassica napus
  source 1..300: mol_type = genomic DNA
atcattatcgcagaaggaggtcggatcagggctgctcaagtccttccttcctaagtaggccataggacgaaactgtttgttcctttgagacagaacatttcttctgctcttgtttttgtaaatcgaaactcctttgaaggctctgatcatagagctgcttcccagcttcactgtggctggggagtcactgccacttgcaccacgagctgatgtgatccatggtgaaacattggagagtctaaaccgatcagaagctccatttccggcagcagaacacgaaaccaaagctctcatctccat

SEQ ID NO: 227 - DNA, 23 bp, synthetic construct
  misc_feature 1..23: note = gRNA
  source 1..23: mol_type = other DNA
tcattatcgcagaaggaggtcgg

SEQ ID NO: 228 - DNA, 23 bp, synthetic construct
  misc_feature 1..23: note = gRNA
  source 1..23: mol_type = other DNA
tcgcagaaggaggtcggatcagg

SEQ ID NO: 229 - DNA, 23 bp, synthetic construct
  misc_feature 1..23: note = gRNA
  source 1..23: mol_type = other DNA
caagtccttccttcctaagtagg

SEQ ID NO: 230 - DNA, 23 bp, synthetic construct
  misc_feature 1..23: note = gRNA
  source 1..23: mol_type = other DNA
ttccttcctaagtaggccatagg

SEQ ID NO: 231 - DNA, 23 bp, synthetic construct
  misc_feature 1..23: note = gRNA
  source 1..23: mol_type = other DNA
gtaaatcgaaactcctttgaagg

SEQ ID NO: 232 - DNA, 23 bp, synthetic construct
  misc_feature 1..23: note = gRNA
  source 1..23: mol_type = other DNA
gctgcttcccagcttcactgtgg

SEQ ID NO: 233 - DNA, 23 bp, synthetic construct
  misc_feature 1..23: note = gRNA
  source 1..23: mol_type = other DNA
cttcccagcttcactgtggctgg

SEQ ID NO: 234 - DNA, 23 bp, synthetic construct
  misc_feature 1..23: note = gRNA
  source 1..23: mol_type = other DNA
ttcccagcttcactgtggctggg

SEQ ID NO: 235 - DNA, 23 bp, synthetic construct
  misc_feature 1..23: note = gRNA
  source 1..23: mol_type = other DNA
tcccagcttcactgtggctgggg

SEQ ID NO: 236 - DNA, 23 bp, synthetic construct
  misc_feature 1..23: note = gRNA
  source 1..23: mol_type = other DNA
ccacgagctgatgtgatccatgg

SEQ ID NO: 237 - DNA, 23 bp, synthetic construct
  misc_feature 1..23: note = gRNA
  source 1..23: mol_type = other DNA
tgtgatccatggtgaaacattgg

SEQ ID NO: 238 - DNA, 23 bp, synthetic construct
  misc_feature 1..23: note = gRNA
  source 1..23: mol_type = other DNA
ccgatcagaagctccatttccgg

SEQ ID NO: 239 - DNA, 23 bp, synthetic construct
  misc_feature 1..23: note = gRNA
  source 1..23: mol_type = other DNA
tccatggagatatattcgtgagg

SEQ ID NO: 240 - DNA, 23 bp, synthetic construct
  misc_feature 1..23: note = gRNA
  source 1..23: mol_type = other DNA
ccctcacgaatatatctccatgg

SEQ ID NO: 241 - DNA, 23 bp, synthetic construct
  misc_feature 1..23: note = gRNA
  source 1..23: mol_type = other DNA
gatatattcgtgagggtaattgg

SEQ ID NO: 242 - DNA, 23 bp, synthetic construct
  misc_feature 1..23: note = gRNA
  source 1..23: mol_type = other DNA
cttctcagcttcactgtcgacgg

SEQ ID NO: 243 - DNA, 23 bp, synthetic construct
  misc_feature 1..23: note = gRNA
  source 1..23: mol_type = other DNA
ccatggagatatattcgtgaggg

SEQ ID NO: 244 - DNA, 23 bp, synthetic construct
  misc_feature 1..23: note = gRNA
  source 1..23: mol_type = other DNA
cttcgacaagagttttgtctcgg

SEQ ID NO: 245 - DNA, 23 bp, synthetic construct
  misc_feature 1..23: note = gRNA
  source 1..23: mol_type = other DNA
tcaagagtgagaacctttaaagg

SEQ ID NO: 246 - DNA, 23 bp, synthetic construct
  misc_feature 1..23: note = gRNA
  source 1..23: mol_type = other DNA
tcttcgtgttctactggtaatgg

SEQ ID NO: 247 - DNA, 23 bp, synthetic construct
  misc_feature 1..23: note = gRNA
  source 1..23: mol_type = other DNA
ttgggttcttcgtgttctactgg

SEQ ID NO: 248 - DNA, 23 bp, synthetic construct
  misc_feature 1..23: note = gRNA
  source 1..23: mol_type = other DNA
tgtcgaagaaactcctttaaagg

SEQ ID NO: 249 - DNA, 23 bp, synthetic construct
  misc_feature 1..23: note = gRNA
  source 1..23: mol_type = other DNA
tcttgacgaaactcctttgaagg

SEQ ID NO: 250 - DNA, 23 bp, synthetic construct
  misc_feature 1..23: note = gRNA
  source 1..23: mol_type = other DNA
agtagtttgagaaccttcaaagg

SEQ ID NO: 251 - DNA, 23 bp, synthetic construct
  misc_feature 1..23: note = gRNA
  source 1..23: mol_type = other DNA
tcgtgttctactggtaatggagg

SEQ ID NO: 252 - DNA, 23 bp, synthetic construct
  misc_feature 1..23: note = gRNA
  source 1..23: mol_type = other DNA
ggaaaaactgttgtgatccatgg

SEQ ID NO: 253 - DNA, 23 bp, synthetic construct
  misc_feature 1..23: note = gRNA
  source 1..23: mol_type = other DNA
aaacagaggaaactgttgtttgg

SEQ ID NO: 254 - DNA, 23 bp, synthetic construct
  misc_feature 1..23: note = gRNA
  source 1..23: mol_type = other DNA
gggtttaggaaacaaaacagagg

SEQ ID NO: 255 - DNA, 24 bp, synthetic construct
  misc_feature 1..24: note = ACC2 target
  source 1..24: mol_type = other DNA
gattgcctcacgaatatatctcca

SEQ ID NO: 256 - DNA, 24 bp, synthetic construct
  misc_feature 1..24: note = ACC2 target
  source 1..24: mol_type = other DNA
aaactggagatatattcgtgaggc

SEQ ID NO: 257 - DNA, 24 bp, synthetic construct
  misc_feature 1..24: note = primer
  source 1..24: mol_type = other DNA
tctcttcctccttaaaaagccaca

SEQ ID NO: 258 - DNA, 20 bp, synthetic construct
  misc_feature 1..20: note = primer
  source 1..20: mol_type = other DNA
ctaggattcgaaaccagcgt

SEQ ID NO: 259 - DNA, 23 bp, synthetic construct
  misc_feature 1..23: note = Zm-PPR10 wt binding site
  source 1..23: mol_type = other DNA
attgtatccttaaccatttcttt

SEQ ID NO: 260 - DNA, 23 bp, synthetic construct
  misc_feature 1..23: note = Bn-PPR10 wt binding site
  source 1..23: mol_type = other DNA
attgtatcattaactatttcttt

SEQ ID NO: 261 - DNA, 23 bp, synthetic construct
  misc_feature 1..23: note = Zm-PPR10GG mut binding site
  source 1..23: mol_type = other DNA
attgtaggcttaaccatttcttt

SEQ ID NO: 262 - DNA, 1140 bp, synthetic construct
  misc_feature 1..1140: note = Prrn-PPR10GG-GFP-TrbcL
  source 1..1140: mol_type = other DNA
gagctcgctcccccgccgtcgttcaatgagaatggataagaggctcgtgggattgacgtgagggggcagggatggctatatttctgggagttacttctacccgatagagcttagaagttggaagtaataatttcttggttgattgtaggcttaaccatttctttttttttgacacgaggaactcatcatggctagcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaaataaatctagaaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctaaagctt

SEQ ID NO: 263 - DNA, 147 bp, synthetic construct
  misc_feature 1..147: note = tRNA
  source 1..147: mol_type = other DNA
aagtctttacaatgacaatggaaaccgatgtaaagggatgtagcgcagcttggtagcgcgtttgttttgggtacaaaatgtcacaggttcaaatcctgtcatccctatccctaacttgtagttatcgtatcagcagtaacaatagat

SEQ ID NO: 264 - DNA, 2361 bp, Zea mays
  source 1..2361: mol_type = genomic DNA
atggaggccaccggcagggggctgttcccgaacaagcccaccctcccggcggggccgaggaaacggggcccgctcctcccggccgcgcccccgccaccgtccccctcctcgctcccgctcgactcgctcctgctccacctcaccgcgcccgcccccgcgccggcccccgcgccgcggcggtcgcaccagacgccgacgccgccgcactccttcctctcccccgacgcgcaggtgctggtgctcgccatctcctcgcacccgctccccacgctggcggccttcctggcctcccgccgcgacgagctcctccgcgcggacatcacgtccctgctcaaggcgctggagctctcggggcactgggagtgggcgctcgcgctcctccggtgggcaggcaaggagggtgccgccgacgcgtcggcgctcgagatggtcgtccgcgcgctgggccgcgagggccagcacgacgccgtctgcgcgctgctcgacgaaacgccgctcccgccgggctcccgcctcgacgtccgcgcctacaccaccgtgctgcacgcgctctcccgcgcgggccggtacgagcgcgcgctcgagctcttcgccgagctccggcgccagggggtggcgcccacgctcgtcacctacaacgtcgtgctggacgtgtacgggcggatgggccggtcgtggccgcggatcgtcgccctcctcgatgagatgcgcgccgccggggtcgagcccgacggcttcaccgccagcacggtgatcgccgcgtgctgccgcgacgggctggttgacgaggcggtggcgttcttcgaggacctcaaggcccgcggccacgccccgtgcgtcgtcacgtacaacgcgttgctccaggtgttcggcaaggccgggaactacacggaggcgctgcgcgtgctcggggagatggagcagaacggctgccagccagatgctgtgacgtacaacgagctcgccggaacgtacgcccgggctgggttcttcgaggaggctgccaggtgcctggacacaatggcatccaagggtctgttgccaaacgcattcacgtacaacaccgtgatgacagcctatgggaatgttgggaaggtggatgaggcgctcgctctgtttgaccagatgaagaagaccgggttcgtgccgaacgtgaacacgtacaatcttgtccttggcatgcttggcaagaagtcaaggttcacggtgatgctagagatgcttggagagatgtcgaggagcggatgcacaccgaaccgggtaacatggaacacaatgcttgcagtctgtgggaagcgtggcatggaggactacgtcacccgggttctggaggggatgaggtcttgcggggttgaactgagccgagacacctacaacaccctgatagctgcgtacggccggtgtggctcgaggactaatgccttcaagatgtacaacgagatgaccagcgctggattcaccccctgcatcaccacgtacaacgcgttgctgaacgtgctgtcgcggcagggcgactggtccaccgcccagtcgatcgtaagcaaaatgaggaccaaggggttcaagccgaacgagcagtcgtattcgctgctgctccagtgctacgcgaaggggggcaacgtggcagggatagccgcgatcgagaacgaggtgtacggatcaggtgccgttttcccaagctgggtgatcctgaggacccttgtcatcgccaatttcaagtgccggcgactggatggcatggagacggcgtttcaagaggtgaaggccagaggctacaacccggacctcgtgatattcaactccatgctgtccatctacgcgaagaacgggatgtacagcaaggccaccgaggtcttcgactccatcaagcggagcgggctgagccccgacctcatcacctacaacagcctgatggacatgtacgccaagtgcagcgagtcgtgggaggccgagaagatactgaaccagctcaagtgctcccagacgatgaagcccgacgtggtgtcctacaacacggtcataaacgggttctgcaagcaggggctggtgaaagaggcccagagggtcctctcggagatggtcgccgacggcatggccccctgcgccgtgacctaccacacgctcgtcgggggttactccagcctggagatgttcagcgaggccagggaggtcatcggctacatggtccagcacggcctcaagcctatggagctgacctacaggagagtcgtcgagagctactgcagagcgaagcggttcgaggaggctcgcggcttcctgtccgaggtctcggagaccgacctggattttgacaagaaggcgctcgaagcctatatagaggatgcgcagtttggaaggtag

SEQ ID NO: 265 - DNA, 2361 bp, synthetic construct
  misc_feature 1..2361: note = Zm_PPR10 GG
  source 1..2361: mol_type = other DNA
atggaggccaccggcagggggctgttcccgaacaagcccaccctcccggcggggccgaggaaacggggcccgctcctcccggccgcgcccccgccaccgtccccctcctcgctcccgctcgactcgctcctgctccacctcaccgcgcccgcccccgcgccggcccccgcgccgcggcggtcgcaccagacgccgacgccgccgcactccttcctctcccccgacgcgcaggtgctggtgctcgccatctcctcgcacccgctccccacgctggcggccttcctggcctcccgccgcgacgagctcctccgcgcggacatcacgtccctgctcaaggcgctggagctctcggggcactgggagtgggcgctcgcgctcctccggtgggcaggcaaggagggtgccgccgacgcgtcggcgctcgagatggtcgtccgcgcgctgggccgcgagggccagcacgacgccgtctgcgcgctgctcgacgaaacgccgctcccgccgggctcccgcctcgacgtccgcgcctacaccaccgtgctgcacgcgctctcccgcgcgggccggtacgagcgcgcgctcgagctcttcgccgagctccggcgccagggggtggcgcccacgctcgtcacctacaacgtcgtgctggacgtgtacgggcggatgggccggtcgtggccgcggatcgtcgccctcctcgatgagatgcgcgccgccggggtcgagcccgacggcttcaccgccagcacggtgatcgccgcgtgctgccgcgacgggctggttgacgaggcggtggcgttcttcgaggacctcaaggcccgcggccacgccccgtgcgtcgtcacgtacacagcgttgctccaggtgttcggcaaggccgggaactacacggaggcgctgcgcgtgctcggggagatggagcagaacggctgccagccagatgctgtgacgtacaccgagctcgccggaacgtacgcccgggctgggttcttcgaggaggctgccaggtgcctggacacaatggcatccaagggtctgttgccaaacgcattcacgtacaacaccgtgatgacagcctatgggaatgttgggaaggtggatgaggcgctcgctctgtttgaccagatgaagaagaccgggttcgtgccgaacgtgaacacgtacaatcttgtccttggcatgcttggcaagaagtcaaggttcacggtgatgctagagatgcttggagagatgtcgaggagcggatgcacaccgaaccgggtaacatggaacacaatgcttgcagtctgtgggaagcgtggcatggaggactacgtcacccgggttctggaggggatgaggtcttgcggggttgaactgagccgagacacctacaacaccctgatagctgcgtacggccggtgtggctcgaggactaatgccttcaagatgtacaacgagatgaccagcgctggattcaccccctgcatcaccacgtacaacgcgttgctgaacgtgctgtcgcggcagggcgactggtccaccgcccagtcgatcgtaagcaaaatgaggaccaaggggttcaagccgaacgagcagtcgtattcgctgctgctccagtgctacgcgaaggggggcaacgtggcagggatagccgcgatcgagaacgaggtgtacggatcaggtgccgttttcccaagctgggtgatcctgaggacccttgtcatcgccaatttcaagtgccggcgactggatggcatggagacggcgtttcaagaggtgaaggccagaggctacaacccggacctcgtgatattcaactccatgctgtccatctacgcgaagaacgggatgtacagcaaggccaccgaggtcttcgactccatcaagcggagcgggctgagccccgacctcatcacctacaacagcctgatggacatgtacgccaagtgcagcgagtcgtgggaggccgagaagatactgaaccagctcaagtgctcccagacgatgaagcccgacgtggtgtcctacaacacggtcataaacgggttctgcaagcaggggctggtgaaagaggcccagagggtcctctcggagatggtcgccgacggcatggccccctgcgccgtgacctaccacacgctcgtcgggggttactccagcctggagatgttcagcgaggccagggaggtcatcggctacatggtccagcacggcctcaagcctatggagctgacctacaggagagtcgtcgagagctactgcagagcgaagcggttcgaggaggctcgcggcttcctgtccgaggtctcggagaccgacctggattttgacaagaaggcgctcgaagcctatatagaggatgcgcagtttggaaggtag

SEQ ID NO: 266 - DNA, 1415 bp, synthetic construct
  misc_feature 1..1415: note = Phaseolin promoter
  source 1..1415: mol_type = other DNA
ggtcgacggtatcgataagcttgatatcgaattcctgcagcccaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttggataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattactgtgggttgccatggcactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgca

SEQ ID NO: 267 - DNA, 22 bp, Brassica napus
  source 1..22: mol_type = genomic DNA
ggtttagactctccaatgtttc

SEQ ID NO: 268 - DNA, 24 bp, synthetic construct
  misc_feature 1..24: note = Forward Oligo
  source 1..24: mol_type = other DNA
gattgctttgtaacctctcagatt

SEQ ID NO: 269 - DNA, 24 bp, synthetic construct
  misc_feature 1..24: note = Reverse Oligo
  source 1..24: mol_type = other DNA
aaacaatctgagaggttacaaagc

SEQ ID NO: 270 - DNA, 22 bp, Brassica napus
  source 1..22: mol_type = genomic DNA
ggaaggaaggacttgagcagcc

SEQ ID NO: 271 - DNA, 24 bp, synthetic construct
  misc_feature 1..24: note = Forward Oligo
  source 1..24: mol_type = other DNA
gattgccgacgagttcaggaagga

SEQ ID NO: 272 - DNA, 24 bp, synthetic construct
  misc_feature 1..24: note = Reverse Oligo
  source 1..24: mol_type = other DNA
aaactccttcctgaactcgtcggc

SEQ ID NO: 273 - DNA, 22 bp, Brassica napus
  source 1..22: mol_type = genomic DNA
ggtgaaacattggagagtctaa

SEQ ID NO: 274 - DNA, 24 bp, synthetic construct
  misc_feature 1..24: note = Forward Oligo
  source 1..24: mol_type = other DNA
gattgaatctgagaggttacaaag

SEQ ID NO: 275 - DNA, 24 bp, synthetic construct
  misc_feature 1..24: note = Reverse Oligo
  source 1..24: mol_type = other DNA
aaacctttgtaacctctcagattc

SEQ ID NO: 276 - DNA, 22 bp, Brassica napus
  source 1..22: mol_type = genomic DNA
ggagcttctgatcggtttagac

SEQ ID NO: 277 - DNA, 22 bp, Brassica napus
  source 1..22: mol_type = genomic DNA
ggtgcaagtggcagtgactccc

SEQ ID NO: 278 - DNA, 22 bp, Brassica napus
  source 1..22: mol_type = genomic DNA
gacaccgacccctcagtgacgg

SEQ ID NO: 279 - DNA, 22 bp, Brassica napus
  source 1..22: mol_type = genomic DNA
ggagtttcgatttacaaaaaca

SEQ ID NO: 280 - DNA, 22 bp, Brassica napus
  source 1..22: mol_type = genomic DNA
ggcctacttaggaaggaaggac

SEQ ID NO: 281 - DNA, 22 bp, Brassica napus
  source 1..22: mol_type = genomic DNA
ggaaggacttgagcagccctga

SEQ ID NO: 282 - DNA, 23 bp, Brassica napus
  source 1..23: mol_type = genomic DNA
gtcctatggcctacttaggaagg

SEQ ID NO: 283 - DNA, 22 bp, Brassica napus
  source 1..22: mol_type = genomic DNA
ggacttgagcagccctgatccg

SEQ ID NO: 284 - DNA, 22 bp, Brassica napus
  source 1..22: mol_type = genomic DNA
atggcctacttaggaaggaagg

SEQ ID NO: 285 - DNA, 22 bp, Brassica napus
  source 1..22: mol_type = genomic DNA
cgacctccttctgcgataatgg

SEQ ID NO: 286 - DNA, 22 bp, Brassica napus
  source 1..22: mol_type = genomic DNA
ggagagtctaaaccgatcagaa

SEQ ID NO: 287 - DNA, 22 bp, Brassica napus
  source 1..22: mol_type = genomic DNA
ggagtcactgccacttgcacca

SEQ ID NO: 288 - DNA, 23 bp, Brassica napus
  source 1..23: mol_type = genomic DNA
ggggagtcactgccacttgcacc

SEQ ID NO: 289 - DNA, 23 bp, Brassica napus
  source 1..23: mol_type = genomic DNA
ggctggggagtcactgccacttg

SEQ ID NO: 290 - DNA, 22 bp, Brassica napus
  source 1..22: mol_type = genomic DNA
ggtcttgtttttgtaaatcgaa

SEQ ID NO: 291 - DNA, 22 bp, Brassica napus
  source 1..22: mol_type = genomic DNA
tccttcctaagtaggccatagg

SEQ ID NO: 292 - DNA, 22 bp, Brassica napus
  source 1..22: mol_type = genomic DNA
aagtccttccttcctaagtagg

SEQ ID NO: 293 - DNA, 22 bp, Brassica napus
  source 1..22: mol_type = genomic DNA
ggctgctcaagtccttccttcc

SEQ ID NO: 294 - DNA, 22 bp, Brassica napus
  source 1..22: mol_type = genomic DNA
gcagaaggaggtcggatcaggg

SEQ ID NO: 295 - DNA, 22 bp, Brassica napus
  source 1..22: mol_type = genomic DNA
gggctgctcaagtccttccttc

SEQ ID NO: 296 - DNA, 22 bp, Brassica napus
  source 1..22: mol_type = genomic DNA
cgcagaaggaggtcggatcagg

SEQ ID NO: 297 - DNA, 22 bp, Brassica napus
  source 1..22: mol_type = genomic DNA
cattatcgcagaaggaggtcgg

SEQ ID NO: 298 - DNA, 22 bp, Brassica napus
  source 1..22: mol_type = genomic DNA
ggtcggatcagggctgctcaag

SEQ ID NO: 299 - DNA, 22 bp, Brassica napus
  source 1..22: mol_type = genomic DNA
ggaggtcggatcagggctgctc

SEQ ID NO: 300 - DNA, 22 bp, Brassica napus
  source 1..22: mol_type = genomic DNA
agcaaaccattatcgcagaagg

SEQ ID NO: 301 - protein, 197 aa, Arabidopsis thaliana
MEMRALGSSCSTGNGGSAPITLTNISPWITTVFPSTVKLRSSLRTFKGVSSRVRTFKGVSSTRVLSRTKQQFPLFCFLNPDPISFLENDVSEAERTVVLPDGSVNGAGSVNGYHSDVVPGRNVAEVNEFCKALGGKRPIHSILVATNGMAAVKFIRSVRTWAYETFGSEKAVKLVAMATPEDMRINAEHIRIADQFV

SEQ ID NO: 302 - protein, 92 aa, synthetic construct
  REGION 1..92: note = consensus sequence
  SITE 20: note = Xaa is any one of N,D,Q, E, B, and Z.
  SITE 20: note = Xaa is any one of N, D, Q, E, B, and Z.
  SITE 22: note = Xaa is any one of N,D,Q, E, B, and Z.
  SITE 39: note = Xaa is I or V.
  SITE 67: note = Xaa is I or V.
GAGSVNGNHSAVGPGRNVAXVXEFCKALRGKRPIHSILXANNGMAAVKFIRSVRTWAYETFGSEKAXLLVAMATPEDMRINAEHIRIADQFV

SEQ ID NO: 303 - protein, 200 aa, Arabidopsis thaliana
MAGSVNGNHSAVGPGINYETVSQVDEFCKALRGKRPIHSILIANNGMAAVKFIRSVRTWAYETFGTEKAILLVGMATPEDMRINAEHIRIADQFVEVPGGTNNNNYANVQLIVEMAEVTRVDAVWPGWGHASENPELPDALDAKGIIFLGPPASSMAALGDKIGSSLIAQAADVPTLPWSGSHVKIPPNSNLVTIPEEIY

SEQ ID NO: 304 - protein, 200 aa, Arabidopsis lyrata
MAGSVNGYQSAIGPGINYETVSQVDEFCKALGGKRPIHSILIANNGMAAVKFIRSVRTWAYETFGTEKAILLVGMATPEDMRINAEHIRIADQFVEVPGGTNNNNYANVQLIVEMAEVTRVDAVWPGWGHASENPELPDALDAKGIIFLGPPAASMAALGDKIGSSLIAQAADVPTLPWSGSHVKMPPNSNLVTIPEEIY

SEQ ID NO: 305 - protein, 200 aa, Camelina sativa
MAGSVNGYQSAVGPGINYESVSQVDEFCKALGGKRPIHSILIANNGMAAVKFIRSVRTWAYETFGTEKAILLVGMATPEDMRINAEHIRIADQFVEVPGGTNNNNYANVQLIVEMAEVTRVDAVWPGWGHASENPELPDALDAKGIIFLGPPAASMGALGDKIGSSLIAQAADVPTLPWSGSHVKIPPNSNLVTIPEEIY

SEQ ID NO: 306 - protein, 200 aa, Camelina sativa
MAGSVNGYQSAVGPGINYETVSQVDEFCKALGGNRPIHSILIANNGMAAVKFIRSVRTWAYETFGTEKAILLVGMATPEDMRINAEHIRIADQFVEVPGGTNNNNYANVQLIVEMAEVTRVDAVWPGWGHASENPELPDALDAKGIIFLGPPAASMGALGDKIGSSLIAQAADVPTLPWSGSHVKIPPNSNLVTIPEEIY

SEQ ID NO: 307 - protein, 200 aa, Capsella sp.
MAGSVNGYQSSVGPGINYETVSQVDEFCKSLGGKRPIHSILIANNGMAAVKFIRSVRTWAYETFGTEKAILLVGMATPEDMRINAEHIRIADQFVEVPGGTNNNNYANVQLIVEMAEVTRVDAVWPGWGHASENPELPDALDAKGIIFLGPPAASMAALGDKIGSSLIAQAADVPTLPWSGSHVKIPPNSNLVTIPEEIY

SEQ ID NO: 308 - protein, 197 aa, Brassica oleracea
MAGSVNGYQTPGRNHVSVSEVDDFCIALGGKRPIHSILIANNGMAAVKFIRSVRTWAYETFGTERAILLVGMATPEDMRINAEHIRIADQFVEVPGGTNNNNYANVQLIVEMAEVTRVDAVWPGWGHASENPELPDALKAKGIIFLGPPAASMAALGDKIGSSLIAQAADVPTLPWSGSHVKIPPDSSLVTIPEEIY

SEQ ID NO: 309 - protein, 197 aa, Brassica napus; identical to SEQ ID NO: 308

SEQ ID NO: 310 - protein, 197 aa, Brassica napus; identical to SEQ ID NO: 308

SEQ ID NO: 311 - protein, 197 aa, Brassica rapa; identical to SEQ ID NO: 308

SEQ ID NO: 312 - protein, 197 aa, synthetic construct
  REGION 1..197: note = consensus sequence
  SITE 20: note = Xaa is any one of N, D, Q, E, B, and Z.
  SITE 23: note = Xaa is any one of N, D, Q, E, B, and Z.
  SITE 186: note = Xaa is any one of N, D, Q, E, B, and Z.
MAGSVNGYQTPGRNHVSVSXVDXFCIALGGKRPIHSILIANNGMAAVKFIRSVRTWAYETFGTERAILLVGMATPEDMRINAEHIRIADQFVEVPGGTNNNNYANVQLIVEMAEVTRVDAVWPGWGHASENPELPDALKAKGIIFLGPPAASMAALGDKIGSSLIAQAADVPTLPWSGSHVKIPPXSSLVTIPEEIY

SEQ ID NO: 313 - protein, 279 aa, Arabidopsis thaliana
MEMRALGSSCSTGNGGSAPITLTNISPWITTVFPSTVKLRSSLRTFKGVSSRVRTFKGVSSTRVLSRTKQQFPLFCFLNPDPISFLENDVSEAERTVVLPDGSVNGAGSVNGYHSDVVPGRNVAEVNEFCKALGGKRPIHSILVATNGMAAVKFIRSVRTWAYETFGSEKAVKLVAMATPEDMRINAEHIRIADQFVEVPGGTNNNNYANVQLIVEMAEVTRVDAVWPGWGHASENPELPDALKEKGIIFLGPPADSMIALGDKIGSSLIAQAADVPTL

SEQ ID NO: 314 - protein, 282 aa, Arabidopsis lyrata
MEMRALVSSCATGNGGSDPFSFTKVSPWITTVGGKDRDFPTTVKLRTSMRTFKGVSIRGRTFKGVSTRVLSRNKQQFPLFCFLNPDPTSFRDNDISEAQRTVVLPGGSVNGYHQSEVVPGRNDGTVAEVDEFCKALGGKRPIHSILVATNGMAAVKFIRSIRTWAYETFGTEKAVKLVAMATPEDMRINAEHIRIADQFVEVPGGTNNNNYANVQLIVEMAEVTRVDAVWPGWGHASENPELPDALKAKGIIFLGPPAASMIALGDKIGSSLIAQAADVPTL

SEQ ID NO: 315 - protein, 280 aa, Camelina sativa
MEMRALVSSYSTGNGGSDPISLTNGSPWITTVGGGASTMDREFPLTVKLGSSMRAFKGVSTTTVLSRTKQQFPLVCLARNNANSTDPTSFWENDISEVQRTVLPAESINGDKSAVEPGRNDVTVSEVDEFCKALGGKRPIHSILVATNGMASVKFIRSIRTWAYQTFGSEKAISLVAMATPEDMRINAEHIRIADQFVEVPGGTNNNNYANVQLIVEMAEATRVDAVWPGWGHASENPELPDALKAKGIIFLGPPATSMVALGDKIGSSLIAQAADVPTL

SEQ ID NO: 316 - protein, 280 aa, Camelina sativa
MEMRALVSSCSTGNGGSDPISLTNGSPWITTVGGGASTMDREFPATVKLGSSMRAFKGVSTITVLSRTKQQFPLVCLARNNGNSTDPTSFWENDISETQRTVLPAESINGDKSAVEPGRNDVTVSEVDEFCKALGGKRPIHRIMVATNGMAAVKFIRSIRTWAYQTFGSEKAISLVAMATPEDMRINAEHIRIADQFVEVPGGTNNNNYANVQLIVEMAEATRVDAVWPGWGHASENPELPDALKAKGIIFLGPPATSMVALGDKIGSSLIAQAADVPTL

SEQ ID NO: 317 - protein, 277 aa, Capsella sp.
MEMRALVSSCSTGNGGSDPISLTNVSPWITTVGGGASSIDREFPTTVKLGSSLRTFKGVSSTTVLSRTKQQFPLVCLARNNANSTDPTLFWENDISEAQSTVLPSGSDESAVVPSGNDVKVSEVDEFCKALGGKRPIHSILVATNGMAAVKFIRSIRTWAYQTFGTEKAILLVAMATPEDMRINAEHIRIADQFVEVPGGTNNHNYANVQLIVEMAEAASVDAVWPGWGHASENPELPDALKAKGIIFLGPSAASMVALGDKIGSSLIAQAADVPTL

SEQ ID NO: 318 - protein, 276 aa, Brassica oleracea
MEMRALVSCSAAGNGASDRFRLSNVSPWITSARGASGSDSPATVKLRSSSMIRAFKGVSIYKNKTRRNVLSQRNKQFRPMAYLGRKDLSSPDPTSFCDNDISEPQGTGSINGNDHSAVRVSQVDEFCKAHGGKRPIHSILVATNGMAAVKLIRSVRAWSCQTFGSEKSISLVAMATPEDMRINAEHIRIADQFMQVPGGTNNNNYANVHLIVEMAQATGVDAVWPGWGHASENPELPDALKAKGVIFLGPTAASMLALGDKIGSSLIAQAADVPTL

SEQ ID NO: 319 - protein, 276 aa, Brassica napus
MEMRALVSCSAAGNGASDRFRLSNVSPWITSARGASGSDSPATVKLGSSSMIRAFKGVSIYKNKTRRNVLSQRNKQFRPMAYLGRKDLSSPDPTSFCDNDISEPQGTGSINGNDHSAVRVSQVDEFCKAHGGKRPIHSILVATNGMAAVKLIRSVRAWSYQTFGSEKSISLVAMATPEDMRINAEHIRIADQFMQVPGGTNNNNYANVHLIVEMAQATGVDAVWPGWGHASENPELPDALKAKGVIFLGPTAASMLALGDKIGSSLIAQAADVPTL

SEQ ID NO: 320 - protein, 276 aa, Brassica napus
MEMRALVSCSAAGNGASDRFRLSNVSPWITSARGASGSDSPATVKLGSSSMIRAFKGVSIYKNKTRRNVLSQRNKQFRPMAYLGRKDLSSPDPTSFCDNDISEPQGTGSINGNDHSAVRVSQVDEFCKAHGGKRPIHRILVATNGMAAVKFIRSVRAWSYQTFGSEKSISLVAMATPEDMRINAEHIRIADQFMQVPGGTNNNNYANVHLIVEMAEATGVDAVWPGWGHASENPELPDALKAKGVIFLGPTAASMLALGDKIGSSLIAQAADVPTL

SEQ ID NO: 321 - protein, 276 aa, Brassica rapa
MEMRALVSCSAAGNGASDRFRLSNVSPWITSARGASGSDSPATVKLGSSSMIRAFKGVSIYKNKSRRNVLSQRNKQFRPMAYLGRKDLSSPDPTSFCDNDISEPQGTGSINGNDHSAVRVSQVDEFCKAHGGKRPIHRILVATNGMAAVKFIRSVRAWSYQTFGSEKSISLVAMATPEDMRINAEHIRIADQFMQVPGGTNNNNYANVHLIVEMAEATGVDAVWPGWGHASENPELPDALKAKGVIFLGPTAASMLALGDKIGSSLIAQAADVPTL

SEQ ID NO: 322 - protein, 254 aa, synthetic construct
  REGION 1..254: note = consensus sequence
  SITE 62: note = Xaa is L or M.
  SITE 73: note = Xaa is any one of N, D, Q, E, B, and Z.
  SITE 76: note = Xaa is I or V.
  SITE 79: note = Xaa is any one of N, D, Q, E, B, and Z.
  SITE 101: note = Xaa is any one of N, D, Q, E, B, and Z.
  SITE 103: note = Xaa is any one of N, D, Q, E, B, and Z.
  SITE 134: note = Xaa is I or V.
  SITE 140: note = Xaa is any one of N, D, Q, E, B, and Z.
  SITE 148: note = Xaa is I or V.
  SITE 174: note = Xaa is any one of N, D, Q, E, B, and Z.
  SITE 195: note = Xaa is any one of N, D, Q, E, B, and Z.
  SITE 224: note = Xaa is I or V.
MEMRALVSSCATGNGGSDPFLTNVSPWITTVGDFPTVKLGSSRAFKGVSIKTVLSRKQQFPXCLRSPDPTSFXNDXSEXRTVLPGSINGDHSAVPGRNVSXVXEFCKALGGKRPIHSILVATNGMAAVKFIRSXRTWAYXTFGSEKAXSLVAMATPEDMRINAEHIRIADQFVXVPGGTNNNNYANVQLIVEMAXATRVDAVWPGWGHASENPELPDALKAKGXIFLGPPAASMALGDKIGSSLIAQAADVPTL

SEQ ID NO: 323 - DNA, 24 bp, synthetic construct
  misc_feature 1..24: note = oligonucleotide sequence
  source 1..24: mol_type = other DNA
gcctcacgaatatatctccagttt

SEQ ID NO: 324 - DNA, 104 bp, synthetic construct
  misc_feature 1..104: note = the parental Ws/RLD sequence
  source 1..104: mol_type = other DNA
atggagatgagagctttgggttcttcgtgttctactggtaatggaggttctgctccaattaccctcacgaatatatctccatggatcacaacagtttttccgtc

SEQ ID NO: 325 - DNA, 105 bp, synthetic construct
  misc_feature 1..105: note = Ws-6-2
  source 1..105: mol_type = other DNA
atggagatgaaagctttgggttcttcgtgttctactggtaatggaggttctgctccaattaccctcacgaatatatcttccatggatcacaacagtttttccgtc

SEQ ID NO: 326 - DNA, 101 bp, synthetic construct
  misc_feature 1..101: note = RLD-6-2
  source 1..101: mol_type = other DNA
atggagatgagagctttgggttcttcgtgttctactggtaatggaggttctgctccaattaccctcacgaatatatcttccatggatcacaacagtttttc

SEQ ID NO: 327 - DNA, 105 bp, synthetic construct
  misc_feature 1..105: note = Ws-10-35
  source 1..105: mol_type = other DNA
atggagatgagagctttgggttcttcgtgttctactggtaatggaggttctgctccaattaccctcacgaatatatcttccatggatcacaacagtttttccgtc

SEQ ID NO: 328 - DNA, 96 bp, synthetic construct
  misc_feature 1..96: note = Ws-11-5
  source 1..96: mol_type = other DNA
atggagatgaaagctttgggttcttcgtgttctactggtaatggaggttctgctccaattaccctcacgaatatggatcacaacagtttttccgtc

SEQ ID NO: 329 - DNA, 105 bp, synthetic construct
  misc_feature 1..105: note = Ws-11-28
  source 1..105: mol_type = other DNA
atggagatgagagctttgggttcttcgtgttctactggtaatggaggttctgctccaattaccctcacgaatatatcttccagggatcacaacattttttccgcc

SEQ ID NO: 330 - DNA, 105 bp, synthetic construct
  misc_feature 1..105: note = Ws-6-23
  source 1..105: mol_type = other DNA
atggagatgaaagctttgggttcttcgtgttctactggtaatggaggttctgctccaattaccctcacgaatatccctgcattggaacacattttctttcccctc

SEQ ID NO: 331 - DNA, 105 bp, synthetic construct
  misc_feature 1..105: note = Ws-6-11
  source 1..105: mol_type = other DNA
atggagatgaaagctttgggttcttcgtgttctactggtaatggaggttctgctccaatttccctcaccaatggatctcccctgtttttccaccgtttttccatc

SEQ ID NO: 332 - DNA, 103 bp, synthetic construct
  misc_feature 1..103: note = Ws-6-9
  source 1..103: mol_type = other DNA
atggagatgaaagctttgggttcttcgtgttctactggtaatggaggttctgctccaattaccctcacgaatatatcccaggaaccaaaaaatttttccctcc

SEQ ID NO: 333 - DNA, 94 bp, synthetic construct
  misc_feature 1..94: note = Ws-11-96
  source 1..94: mol_type = other DNA
atggagatgagagctttgggttcttcgtgttctactggtaatggaggttctgctccaattaccctcacgaatatatcacaacagtttttccgtc

SEQ ID NO: 334 - DNA, 105 bp, synthetic construct
  misc_feature 1..105: note = Ws-6-19
  misc_feature 67: note = n is a, c, g, or t
  source 1..105: mol_type = other DNA
atggagatgaaagctttgggttcttcgtgttctactggtaatggaggttctgctccaattaccctcncgaatatatcttccatggatcacaacagtttttccgtc

SEQ ID NO: 335 - DNA, 101 bp, synthetic construct; identical to SEQ ID NO: 326
  misc_feature 1..101: note = RLD-10-25
  source 1..101: mol_type = other DNA

SEQ ID NO: 336 - DNA, 101 bp, synthetic construct; identical to SEQ ID NO: 326
  misc_feature 1..101: note = RLD-6-10
  source 1..101: mol_type = other DNA

SEQ ID NO: 337 - DNA, 99 bp, synthetic construct
  misc_feature 1..99: note = RLD-10-2
  source 1..99: mol_type = other DNA
atggagatgaaagctttgggttcttcgtgttctactggtaatggaggttctgctccaattaccctcacgaatatatctaatggatcacaacagtttttc

SEQ ID NO: 338 - DNA, 101 bp, synthetic construct; identical to SEQ ID NO: 326
  misc_feature 1..101: note = RLD-6-13
  source 1..101: mol_type = other DNA

SEQ ID NO: 339 - DNA, 101 bp, synthetic construct; identical to SEQ ID NO: 326
  misc_feature 1..101: note = RLD-6-15
  source 1..101: mol_type = other DNA

SEQ ID NO: 340 - DNA, 98 bp, synthetic construct
  misc_feature 1..98: note = RLD-10-10
  source 1..98: mol_type = other DNA
atggagatgagagctttgggttcttcgtgttctactggtaatggaggttctgctccaattaccctcacgaatatatccatggatcacaacagtttttc

SEQ ID NO: 341 - DNA, 101 bp, synthetic construct; identical to SEQ ID NO: 326
  misc_feature 1..101: note = RLD-6-6
  source 1..101: mol_type = other DNA

SEQ ID NO: 342 - DNA, 98 bp, synthetic construct; identical to SEQ ID NO: 340
  misc_feature 1..98: note = RLD-10-29
  source 1..98: mol_type = other DNA

SEQ ID NO: 343 - DNA, 101 bp, synthetic construct
  misc_feature 1..101: note = RLD-11-14
  misc_feature 94: note = n is a, c, g, or t
  source 1..101: mol_type = other DNA
atggagatgagagctttgggttcttcgtgttctactggtaatggaggttctgctccaattaccctcaccaagaatccctccatgttttccaacnacttttc

SEQ ID NO: 344 - DNA, 101 bp, synthetic construct
  misc_feature 1..101: note = RLD-10-8
  source 1..101: mol_type = other DNA
atggagatgagagctttgggttcttcgtgttctactggtaatggaggttctgctccaattaccctcacgaatatatcctcggaggatcacaattttttttc

SEQ ID NO: 345 - DNA, 399 bp, synthetic construct
  misc_feature 1..399: note = N-terminal nucleotides of Brassica napus cv Darmor-bzh ACC2-Br: BnaA06g04070D
  source 1..399: mol_type = other DNA
atggagatgagagctttggtttcgtgttctgctgccggaaatggagcttctgatcggtttagactctccaatgtttcaccatggatcacatcagctcgtggtgcaagtggcagtgactccccagccacagtgaagctgggaagcagctctatgatcagagccttcaaaggagtttcgatttacaaaaacaagaccagaagaaatgttttgtctcaaaggaacaaacagtttcgtcctatggcctacttaggaaggaaggacttgagcagccctgatccgacctccttctgcgataatgatatatctgaacctcaagggactggatccattaatgggaatgatcatagtgctgtaagagtgtctcaagtcgatgagttctgtaaggctcacggtggaaaa

SEQ ID NO: 346 - DNA, 399 bp, synthetic construct
  misc_feature 1..399: note = N-terminal nucleotides of Brassica napus cv Darmor-bzh ACC2-Bo: BnaC06g01580D
  source 1..399: mol_type = other DNA
atggagatgagagctttagtttcgtgttctgctgccggaaatggagcttctgatcggtttagactctccaatgtttcaccatggatcacatcagctcgtggtgcaagtggcagtgactccccagccacagtgaagctgggaagcagctctatgattagagctttcaaaggagtttcgatttacaaaaacaagaccagaaggaatgttctgtctcaaaggaacaaacagttccgtcctatggcctacttaggaaggaaggacttgagcagccctgatccgacctccttctgcgataatgatatatctgaacctcaagggaccggatccattaatgggaatgatcatagtgctgtaagagtgtctcaagtcgatgagttctgtaaggctcatggtggaaaa

SEQ ID NO: 347 - DNA, 93 bp, synthetic construct
  misc_feature 1..93: note = N-terminal nucleotides of Brassica napus cv Darmor-bzh ACC1-Br: BnaA08g06180D
  source 1..93: mol_type = other DNA
atggctggctctgttaacgggtatcaaactcccggtagaaatcatgtttcggtgtctgaagtggatgacttttgcattgcacttggagggaaa

SEQ ID NO: 348 - DNA, 93 bp, synthetic construct; identical to SEQ ID NO: 347
  misc_feature 1..93: note = N-terminal nucleotides of Brassica napus cv Darmor-bzh ACC1-Bo: BnaC08g06560D
  source 1..93: mol_type = other DNA

SEQ ID NO: 349 - DNA, 22 bp, synthetic construct
  misc_feature 1..22: note = sgRNA-target
  source 1..22: mol_type = other DNA
ggtttagactctccaatgtttc

SEQ ID NO: 350 - DNA, 22 bp, synthetic construct
  misc_feature 1..22: note = sgRNA-target
  source 1..22: mol_type = other DNA
ggaaggaaggacttgagcagcc

SEQ ID NO: 351 - DNA, 22 bp, synthetic construct
  misc_feature 1..22: note = sgRNA-target
  source 1..22: mol_type = other DNA
ttagactctccaatgtttcacc

SEQ ID NO: 352 - DNA, 298 bp, Brassica napus; identical to SEQ ID NO: 152
  source 1..298: mol_type = genomic DNA

SEQ ID NO: 353 - DNA, 298 bp, Brassica napus; identical to SEQ ID NO: 182
  source 1..298: mol_type = genomic DNA

(19) United States
(12) Patent Application Publication
(10) Pub. No.: US 2024/0182917 A1
(43) Pub. Date: Jun. 6, 2024

(54) COMPOSITIONS AND METHODS FOR IMPROVING PLASTID TRANSFORMATION EFFICIENCY IN HIGHER PLANTS

(71) Applicant: RUTGERS, THE STATE UNIVERSITY OF NEW JERSEY, New Brunswick, NJ (US)

(72) Inventor: Pal Maliga, East Brunswick, NJ (US)

(73) Assignee: RUTGERS, THE STATE UNIVERSITY OF NEW JERSEY, New Brunswick, NJ (US)

(21) Appl. No.: 18/504,766

(22) Filed: Nov. 8, 2023

Related U.S. Application Data
(63) Continuation of application No. 17/532,602, filed on Nov. 22, 2021, now abandoned.
Application No. 17/532,602 is a continuation of application No. 16/506,756, filed on Jul. 9, 2019, now Pat. No. 11,208,665.
Application No. 16/506,756 is a continuation-in-part of application No. PCT/US2018/013034, filed on Jan. 9, 2018.
(60) Provisional application No. 62/444,307, filed on Jan. 9, 2017.

(51) Int. Cl. C12N 15/82

(52) U.S. Cl. CPC C12N 15/8214

(57) ABSTRACT

Compositions and methods for improving plastid transformation in difficult to transform plants are disclosed.

This application is a continuation of U.S. patent application Ser. No. 16/506,756, filed Jul. 9, 2019, which is a continuation-in-part of International Application No. PCT/US2018/013034 filed Jan. 9, 2018 which claims priority under 35 U.S.C. § 119(e) to U.S. Provisional Patent Application No. 62/444,307, filed on Jan. 9, 2017. The entire disclosure of each of the aforesaid applications is incorporated by reference in the present application.

### INCORPORATION-BY-REFERENCE OF MATERIAL SUBMITTED IN ELECTRONIC FORM

Incorporated herein by reference in its entirety is the Sequence Listing submitted via EFS-Web as a text file named 6424US01_20190925_03_SequenceListing_ST25.txt, created Sep. 25, 2019 and is 138,941 bytes in size.

### FIELD OF THE INVENTION

The present invention relates the fields of plant biology and plastid transformation. More specifically, the invention pertains to molecular strategies for improving plastid transformation efficiency in recalcitrant plant species.

### BACKGROUND OF THE INVENTION

Several publications and patent documents are cited throughout the specification in order to describe the state of the art to which this invention pertains. Each of these citations is incorporated by reference herein as though set forth in full.

Plastids are semi-autonomous plant organelles with thousands of copies of the ˜155-kb genome localized in 10 to 100 plastids per cell. The plastid genome of higher plants encodes about one hundred genes, the products of which assemble with ˜3,000 nucleus-encoded proteins to form the plastid transcription and translation machinery and carry out complex metabolic functions, including photosynthesis, and fatty acid and amino acid biosynthesis. Transformation of the plastid genome in flowering plants was first accomplished in tobacco (Nicotiana tabacum), the current model species of plastid engineering (Svab et al., 1990; Svab and Maliga, 1993).

Plastid transformation is routine only in tobacco, but reproducible protocols for plastid transformation have also been described in tomato (Ruf et al., 2001), potato (Valkov et al., 2011), lettuce (Kanamoto et al., 2006; Ruhlman et al., 2010) and soybean (Dufourmantel et al., 2004). Still, the technology is available in only a relatively small number of crops. Arabidopsis thaliana, the most widely used model plant is one of the species that is recalcitrant to plastid transformation. In Arabidopsis, only 2 transplastomic events were identified in 201 samples (Sikdar et al., 1998), a sample size that would have yielded ˜200 events in tobacco using the technology available in 1988 (Svab and Maliga, 1993). Until now the reasons for the low efficiency in Arabidopsis were not understood.

### SUMMARY OF THE INVENTION

In accordance with the present invention, a method for increasing sensitivity to spectinomycin in plastids of higher plants for increasing plastid transformation efficiency is provided. An exemplary method comprises providing a plant having a nonfunctional ACC2 nuclear gene, introducing one or more plastid transformation vectors into plastids in cells from said plant, said one or more vectors comprising an aadA spectinomycin resistance marker sequence and a nucleic acid sequence encoding a protein of interest. The plant cells are then contacted with spectinomycin and spectinomycin resistant plant cells which accumulate the protein of interest in said plastids selected. The method also includes culturing said plant cells under conditions suitable to regenerate a transplastomic plant therefrom. In preferred embodiments, the plant is selected from the group consisting of Arabidopsis ssp., Brassica ssp., Camelina ssp., and Crambe spp. In a further aspect, the method entails excising the resistance marker from said plant. This can be achieved using the protocols provided in U.S. Pat. Nos. 8,841,511; 7,667,093 and 7,217,860.

Plants to be transformed can be naturally occurring ACC2 mutants which are defective in acc2 activity. Alternatively, desirable plant species can be identified and the ACC2 gene is inactivated in said plant using the CRISPR/Cas system and the appropriate guide strands.

In another embodiment, a method for seed-specific plastid expression is provided. An exemplary method comprises introducing a nuclear expression vector encoding a modified PPR10 binding protein driven by a seed-specific promoter and a plastid expression vector encoding a gene of interest linked to an upstream PPR10 binding site, wherein nuclear-expressed PPR10 is imported into plastids and binds said PPR10 binding site to drive expression of the gene of interest in seed plastids. In certain embodiments, the vector comprises a seed specific promoter selected from a napin or a phascolin gene promoter. In other embodiments, the modified PPR10 binding protein is PPR10^{GG} and encoded by SEQ ID NO: 265. The PPR10 binding site may also be encoded by SEQ ID NO: 261. The vector may also comprise the aadA spectinomycin resistance gene. Additionally, in another aspect, the plastid expressed gene of interest is linked to an upstream sequence encoding a maize atpH gene and/or tRNA sequence in said plastid vector.

In another aspect of the invention, a method for increasing sensitivity to plastid translation inhibitors in plastids of higher plants for increasing plastid transformation efficiency is provided. An exemplary method comprises providing a plant comprising a nonfunctional ACC2 nuclear gene, introducing one or more plastid transformation vectors into the plastids in cells from said plant, said one or more vectors comprising a nucleic acid sequence conferring resistance to said plastid translation inhibitor, and a nucleic acid sequence encoding a protein of interest. The method further entails contacting said cells with said inhibitor and selecting plant cells which are resistant to said inhibitor and accumulate said protein of interest in said plastids; and culturing said plant cells under conditions suitable to regenerate a transplastomic plant therefrom. In certain embodiments, the plastid translation inhibitor is selected from kanamycin, chloramphenicol, tobramycin and gentamycin.

### BRIEF DESCRIPTION OF THE DRAWINGS

FIGS. 1A-1B. Defective ACC2 Gene Makes Chloroplasts More Sensitive to Spectinomycin. (FIG. 1A) In most accessions the heteromeric ACCase (hetACC) localizes in the chloroplast and is encoded by nuclear genes CAC1-A (At5g16390; Biotin Carboxyl Carrier Protein 1 (BCCP-1)), CAC1-B (At5g15530; Biotin Carboxyl Carrier Protein 2 (BCCP-2)) (not depicted in figure), CAC2 (At5g35360; Biotin Carboxylase (BC)), CAC3 (At2g38040, α subunit of Carboxyltransferase (α-CT) and the plastid encoded gene accD (AtCg00500; (β subunit of Carboxyltransferase (β-CT)). The homomeric ACC1 (At1g36160; homACCase) enzyme localizes in the cytoplasm and the ACC2 (At1g36180; homACCase) enzyme is imported into the chloroplast via the TIC/TOC membrane protein complex. If translation of the plastid accD mRNA is blocked by spectinomycin, the nuclear homomeric ACC2 gene supplies the cells with lipids so that cellular viability is not affected. (FIG. 1B) In ACC2 mutants, the absence of the homomeric ACCase makes the plants dependent on plastid translation to produce the heteromeric ACCase enzyme for fatty acid biosynthesis.

FIG. 2. Map of the Plastid Genome with the Integrated aadA-gfp Dicistronic Operon. The NruI-XbaI region is contained in the plastid transformation vector pATV1. P and T mark the positions of the PrrnL atpB promoter and the TpsbA terminator in the dicistronic vector. The black box at the aadA N terminus marks the atpB downstream box sequence (Kuroda and Maliga, 2001). The ribosome entry site is marked by black semi-ovals. The positions of the rrn16 and trnV plastid genes and relevant restriction enzyme sites are marked. Thick black and red lines indicate probes used for DNA and RNA gel-blot analyses, respectively.

FIGS. 3A-3F. Identification of Arabidopsis Transplastomic Clones. (FIG. 3A) Sterile Sav-0 plants grown in Petri dishes (diameter 10 cm) for six weeks. (FIG. 3B) Two days after bombardment (biolistic transformation) the leaves are incised and transferred to selective spectinomycin (100 mg/L) medium. (FIG. 3C) Sav-0 leaves on selective medium one month after bombardment. Note scanty callus formation and green cell cluster (arrow). (FIG. 3D) Culture shown in FIG. 3C, illuminated with UV light. Note green fluorescence indicating GFP accumulation in green cell cluster. (FIG. 3E) Sav-0 plant regenerated from a transplastomic clone #6. (FIG. 3F) Culture shown in FIG. 3E, illuminated with UV light. Inset-Sav-0 #3 seed progeny illuminated with UV light. Bar=1 mm.

FIG. 4. Green Fluorescent Protein (GFP) accumulates in chloroplasts. Shown are confocal images collected in the GFP, chlorophyll, and merged channels on a Leica TCS SP5II confocal microscope. Excitation wavelengths were at 488 and 568 nm, and detection was at 500 to 530 and 650 to 700 nm, respectively. Note the absence of GFP and chlorophyll in the wild-type Col-0 callus cells and mixed GFP-expressing transgenic and wild-type plastids in the Col-0 acc2-1 #1 and Sav-0 #6 lines. Note the absence of wild-type plastids in the leaves of Sav-0 #6 plants. Yellow color in the merged images indicates the colocalization of GFP and chlorophyll in plastids. Note that cells in the small green cell clusters are heteroplastomic. The only exception are cells in Sav-0 6 leaves, which are homoplastomic due to prolonged selection in tissue culture. Bars=10 um

FIGS. 5A-5B. Molecular Characterization of the Sav-0 Transplastomic Clones. (FIG. 5A) DNA gel blot using the rrn16 probe (FIG. 2) indicates that the transplastomic Sav-0 calli and leaves are homoplastomic, carrying only the 4.7-kb EcoRI fragment and lacking the 2.7-kb wild type fragment. (FIG. 5B) The aadA and gfp probes recognize the same 2 kb dicistronic mRNA.

FIGS. 6A-6C. Alignment of homomeric ACCases in the Brassicaceae family. (FIG. 6A) Alignment of 200 the N-terminal amino acids of Arabidopsis thaliana ACC1 (At1g36160) and ACC2 (At1g36180) genes. (FIG. 6B) Alignment of 200 the N-terminal amino acids of Arabidopsis thaliana ACC1: At1g36160; Arabidopsis lyrata ACC1: XM_002891166.1; Camelina sativa ACC1-1: LOC104777496; Camelina sativa ACC1-2: LOC104743830; Capsella rubella ACC1: CARUB_v10011872mg; Brassica oleracea ACC1: LOC106311006; Brassica napus ACC1-1: LOC106413885; Brassica napus ACC1-2: LOC106418889; Brassica rapa ACC1: LOC103833578. (FIG. 6C) Alignment of 300 the N-terminal amino acids of Arabidopsis thaliana ACC2: At1g36180; Arabidopsis lyrata ACC2: XM_002891167.1; Camelina sativa ACC2-1: LOC104777495; Camelina sativa ACC2-2: LOC104742086; Capsella rubella ACC2: CARUB_v10008063mg; Brassica oleracea ACC2: LOC106301042; Brassica napus ACC2-1: Y10302; Brassica napus ACC2-2: X77576; Brassica rapa ACC2: LOC103871500.

FIG. 7. Design of sgRNAs for simultaneous mutagenesis of both B. napus ACC2 gene copies. Aligned are the first exons encoding the N-terminal plastid targeting regions. The GG of NGG of the PAM sequence is encircled; the 20 nucleotide forward guide sequence (5′-3′) is marked with a horizontal line. The first nucleotide of the guide sequence should be changed to a G or an A, dependent on the use of U6 or U3 promoter, respectively (Belhaj et al., 2013). 9 of the 15 potential gRNA sequences are suitable for targeting both ACC2 copies (2-8 and 14,15). The reverse guide sequences are included in Table 3.

FIG. 8. Mutations generated by CRISPR/Cas9 mutagenesis in the Arabidopsis Wassiliewskija (Ws) and RLD ecotypes. Top—Columbia reference sequence and the parental Ws/RLD sequences. Note mutations that alter the reading frame yielding non-functional protein, such as a one nucleotide insertion in Ws-6-2 and RLD-6-2 lines. Bottom—oligonucleotide sequence used for construction of gRNA.

FIG. 9. Ws T3 seed germinated on 100 mg/L spectinomycin medium testing for hypersensitive response. After 2 weeks, the wild-type Ws seedlings bleach but develop primary leaves, in contrast to Ws-2-22 homozygous ACC2 knock-out seedlings which germinate, but do not develop shoot meristem outgrowths on spectinomycin.

FIG. 10. Schematic design of a Brassica napus plastid transformation vector is shown. The plastid targeting sequence comprises the rrn16 targeting region (nucleotides 135473-137978 in GenBank accession KP161617). The vector carries a target site flanked selectable aadA marker gene. The recombinase target sites are marked with triangles. The marker gene and gene of interest have different promoters (P1, P2) and terminators (T1, T2) to avoid deletions by recombination via duplicates sequences.

FIG. 11. A schematic diagram depicting system for seed-specific expression of plastid genes from acc2 defective plants.

FIGS. 12A-12B. Transgenes for seed-specific expression in Brassica spp. (FIG. 12A) Plastid transgenes. P1 and T1 are the expression signals of the aadA marker gene. Preferred sequences are listed in text. P1 is the tobacco plastid Prrn sequence. The half circle is the maize sequence containing BS^{ZmGG} sequence. gfp encodes green fluorescence protein. T1 is the rbcL gene terminator. Cloverleaf symbolizes tRNA gene. (FIG. 12B) The map of Agrobacterium binary vector pCAMBIA2300 with the PnpaA:Zm-PPR10GG:Tocs and selectable kanamycin resistance gene. P1 and T1 are Pnos/Tnos, the expression signals of kanamycin resistance (neo) gene. P2 is the PnpaA napin promoter; PPR10^{GG} sequence is the mutant maize PPR10 protein coding sequence; T2 is Tocs octopine synthase transcription terminator. LB and RB are the T-DNA left and right border sequences.

FIG. 13. Alignment of the N-terminal nucleotides of Brassica napus cv Darmor-bzh ACC2-Br: BnaA06g04070D; ACC2-Bo: BnaC06g01580D; ACC1-Br: BnaA08g06180D; ACC1-Bo: BnaC08g06560D.

FIG. 14A-14C. FIG. 14A Functional ACC2 copies make B. napus plants tolerant to spectinomycin, permitting growth beyond the cotyledon stage. FIG. 14B and FIG. 14C. Flowchart to obtain Cas9-free spectinomycin hypersensitive acc2 Brassica napus. (14B) Selection of CRISPR/Cas9 transgenic plants by kanamycin resistance. (14C) Hypersensitivity bioassay identifies T1 families with putative knockouts in all ACC2 copies, leading to the isolation of Cas9-free acc2 individuals. Non-uniform hypersensitivity to spectinomycin will prompt an additional cycle of screening in the next seed generation.

### DETAILED DESCRIPTION OF THE INVENTION

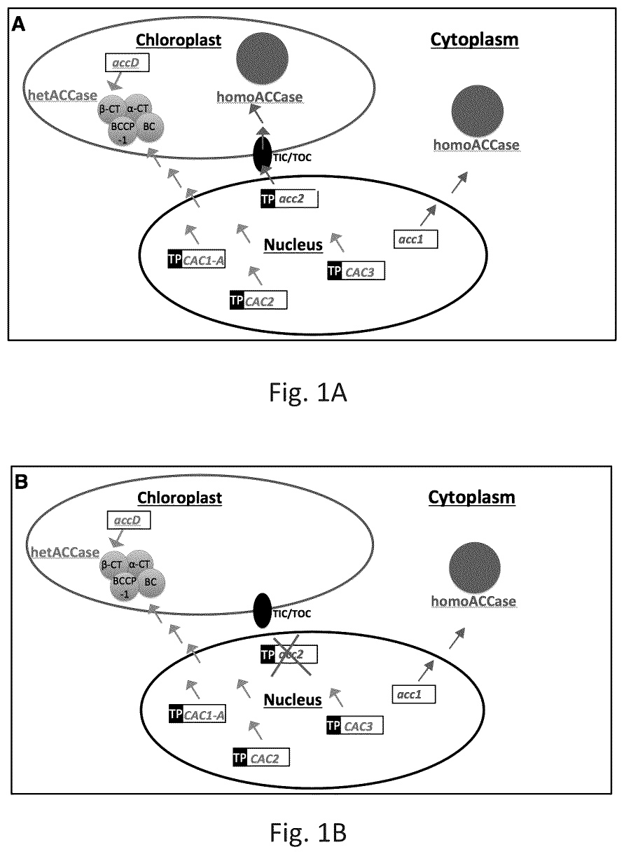

Spectinomycin, a preferred agent used for selecting for transplastomic events, binds to the 16S ribosomal RNA, blocking translation on the prokaryotic type 70S plastid ribosomes (Wirmer and Westhof, 2006; Wilson, 2014) inhibiting greening and shoot regeneration in tissue culture cells (Svab et al., 1990). When the plastid genome is transformed with the aadA gene encoding aminoglycoside-3″-adenylyltransferase, the modified antibiotic no longer binds to the 16S rRNA and translation proceeds, enabling greening. Tobacco, when cultured on a spectinomycin medium, bleaches and proliferates at a slow rate due to inhibition of plastid translation. Transplastomic tobacco cells are identified in tissue culture by the ability to green and regenerate shoots. In contrast, Arabidopsis bleaches but continues to proliferate on a spectinomycin medium in the absence of chloroplast ribosomes (Zubko and Day, 1998). Two major studies by Parker et al. (Parker et al., 2014, 2016) revealed the existence of rare Arabidopsis accessions, in which plastids are extremely sensitive to spectinomycin. Seeds of most accessions in the study germinated on spectinomycin and developed into albino plants. However, in certain accessions, spectinomycin blocked plant development: the seeds germinated, but did not develop beyond the cotyledonary stage. Genetic analysis revealed that spectinomycin sensitivity in these accessions is due to mutations in the ACC2 nuclear gene. The ACC2 gene produces the homomeric acetyl-CoA-carboxylase (ACCase) that is imported into plastids, and duplicates the function of heteromeric ACCase, one subunit of which is encoded in the plastid accD gene (FIG. 1A). When plastid translation is blocked by spectinomycin and no heteromeric ACCase is made, the homomeric enzyme enables a limited amount of fatty acid biosynthesis and development of albino plants. In the absence of a functional ACC2 gene, fatty acid biosynthesis is dependent on the availability of heteromeric ACCase enzyme, the β-Carboxylase subunit of which is translated on plastid ribosomes (FIG. 1B).

We hypothesized that the inefficiency of plastid transformation observed in our early efforts with Arabidopsis was due to the lack of the sensitivity to spectinomycin, and that transformation of mutants defective in ACC2 function should increase efficient recovery of transplastomic clones. We report here that the efficiency of plastid transformation in the acc2 background in Arabidopsis is comparable to that of tobacco, confirming our hypothesis. Antibiotics kanamycin, chloramphenicol, tobramycin and gentamycin are similar to spectinomycin in that they also act through inhibition of plastid translation. Kanamycin resistance is conferred by the neo (nptlI) gene, encoding neomycin phosphotransferase or the aphA-6 gene encoding an aminoglycoside phosphotransferase. Chloramphenicol resistance is conferred by the cat gene encoding chloramphenicol acetyltransferase. Tobramycin/gentamycin resistance is conferred by the bifunctional aac(6′)-Ie/aph(2″)-Ia gene, abbreviated as aac6-aph2 gene, encoding the bifunctional aminoglycoside phosphotransferase(6′)-Ie/APH(2″)-Ia enzyme.

Thus, improved recovery of transplastomic events is expected in the acc2 defective background using these inhibitors of organellar translation as selective markers.

In view of this finding, we have expanded our efforts to create additional strains of acc2 defective plants in the Brassicaceae family. Herein below protocols and expression vectors are provided for both nuclear and plastid transformation in such plants, which include, without limitation, A. lyrata, C. sativa, C. ruella, B. oleracea, B. napus, B. rapa. The inventor also provides suitable guide strands for introducing mutations in ACCases via a CRISPR/CAS.

The definitions below are provided to facilitate an understanding of the invention.

Heteroplastomic refers to the presence of a mixed population of different plastid genomes within a single plastid or in a population of plastids contained in plant cells or tissues.

Homoplastomic refers to a pure population of plastid genomes, either within a plastid or within a population contained in plant cells and tissues. Homoplastomic plastids, cells or tissues are genetically stable because they contain only one type of plastid genome. Hence, they remain homoplastomic even after the selection pressure has been removed, and selfed progeny are also homoplastomic. For purposes of the present invention, heteroplastomic populations of genomes that are functionally homoplastomic (i.e., contain only minor populations of wild-type DNA or transformed genomes with sequence variations) may be referred to herein as “functionally homoplastomic” or “substantially homoplastomic.” These types of cells or tissues can be readily purified to a homoplastomic state by continued selection.

Plastome refers to the genome of a plastid.

Transplastome refers to a transformed plastid genome.

Transformation of plastids refers to the stable integration of transforming DNA into the plastid genome that is transmitted to the seed progeny of plants containing the transformed plastids. Transient expression of heterologous DNA into the plastid or nuclear compartments can also be employed.

Selectable marker gene refers to a gene that upon expression confers a phenotype by which successfully transformed plastids or cells or tissues carrying the transformed plastid can be identified.

Transforming DNA refers to homologous DNA, or heterologous DNA flanked by homologous DNA, which when introduced into plastids becomes part of the plastid genome by homologous recombination.

“Operably linked” refers to two different regions or two separate genes spliced together in a construct such that both regions will function to promote gene expression and/or protein translation.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The term “functional” as used herein implies that the nucleic or amino acid sequence is functional for the recited assay or purpose.

Mao et al. provide detailed guidance for use of the CRISPR/Cas system in higher plants in Molecular Plant, 6: 2008-2011 (2013). The article entitled “Application of the CRISPR-Cas System for Efficient Genome Engineering in Plants” and its supplemental material is incorporated herein by reference as though set forth in full.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion, biolistic bombardment and the like.

“Floral dip transformation” refers to Agrobacterium mediated DNA transfer, in which the flower is brought in contact with the Agrobacterium solution. Floral dip transformation has been described in Arabidopsis (Clough and Bent, 1998) and Brassica spp. (Verma et al., 2008; Tan et al., 2011).

“T-DNA” refers to the transferred-region of the Ti (tumor-inducing) plasmid of Agrobacterium tumefaciens. Ti plasmids are natural gene transfer systems for the introduction of heterologous nucleic acids into the nucleus of higher plants. Binary Agrobacterium vectors such pBIN20 and pPZP222 (GenBank Accession Number U10463.1) are known in the art.

A “plastid transit peptide” is a sequence which, when linked to the N-terminus of a protein, directs transport of the protein from the cytoplasm to the plastid.

A “clone” or “clonal cell population” is a population of cells derived from a single cell or common ancestor by mitosis.

A “cell line” is a clone of a primary cell or cell population that is capable of stable growth in vitro for many generations.

A “defective” and “nonfunctional” gene, such as acc2, refers to a gene which does not encode a functional protein. For example, a one nucleotide insertion on deletion may alter the reading frame to creates an in-frame stop codon.

### Methods for Creating Transplastomic Plants Using the Compositions of the Invention

Virtually all dicots have accD, an heteromeric ACCase subunit gene encoded in their plastid genome, but also have homomeric, plastid targeted nuclear ACC2 gene copies, which is the likely cause for the difficulty of extending the plastid transformation technology to all crops. Deletion of the nuclear ACC2 genes will enable plastid transformation in these dicot species and genetic lines.

The recognition that the plastid targeted ACCase in Arabidopsis is an impediment to plastid transformation provides a rational template to implement plastid transformation in recalcitrant crops. The accD gene is present on the plastid genome of most crops. The Arabidopsis thaliana ACC2 enzyme has an N-terminal extension relative to ACC1 that serves as an N-terminal plastid targeting sequence (Babijchuk et al., 2011). The ACC1 and ACC2 genes are present in all Brassicaceae species, including Arabidopsis lyrata, Camelina sativa, Camelina rubella, Brassica oleracea, Brassica napus and Brassica rapa. The homomeric ACC2 enzyme in these species has an N-terminal extension relative to ACC1. A targeted mutation in the N-terminal extension should selectively inactivate the ACC2 variant, expected to create a spectinomycin sensitive mutant similar to the Col-0 acc2-1 mutant derivative (Parker et al., 2014). Plastid transformation has been achieved in cabbage (Brassica oleracea L. var. capitata L.), thus knockout of ACC2 is apparently not necessary to obtain transplastomic events in this crop, at least in the two cultivars tested (Liu et al., 2007; Liu et al., 2008). Plastid transformation in cauliflower (Brassica oleracea var. botrytis) has been obtained, but at a very low frequency (Nugent et al., 2006). Plastid transformation in oilseed rape (Brassica napus) has also been obtained, but no homoplastomic plants could be obtained (Hou et al., 2003; Cheng et al., 2010), or the transformation efficiency was low (Schneider et al., 2015). Plastid transformation in Lesquerella fendleri, another oilseed crop in the Brassicaceae, was feasible but inefficient (Skarjinskaia et al., 2003). Mutagenesis of ACC2 in the latter cases should significantly boost plastid transformation efficiency. Accordingly, a CRISPR/Cas approach for knocking out the ACC gene is provided in Example II.

Alternatively, desirable plant species could be screened for mutations in nuclear ACC genes and those strains harboring such mutations utilized in the plastid transformation methods disclosed herein. Such strains should inherently be more sensitive to spectinomycin.

The materials and methods set forth below were utilized in the performance of Example I.

### Tissue Culture Media

The tissue culture media were adopted from Sikdar et al. (1998), originally described by Márton and Browse (1991). The culture media are based on Murashige and Skoog (MS) salts (Murashige and Skoog, 1962). ARM consists of MS salts, 3% (w/v) Suc. 0.8% (w/v) agar (A7921; Sigma), 200 mg of myoinositol, 0.1 mg of biotin (1 mL of 0.1 mg mL−1 stock), and 1 mL of vitamin solution (10 mg of vitamin B1, 1 mg of vitamin B6, 1 mg of nicotinic acid, and 1 mg of Gly per mL) per liter, pH 5.8. ARM5 medium consists of ARM supplemented with 5% (w/v) Suc. ARMI medium consists of ARM containing 3 mg of IAA, 0.6 mg of benzyladenine, 0.15 mg of 2,4-D, and 0.3 mg of isopentenyladenine per liter. ARMIIr medium consists of ARM supplemented with 0.2 mg/L naphthaleneacetic acid and 0.4 mg of isopentenyladenine per liter. The stocks of filter-sterilized plant hormones and antibiotics (100 mg/L spectinomycin HCl) were added to media cooled to 45° C. after autoclaving.

Shoot regeneration in the transplastomic Sav-0 clones was obtained on an ARM containing 2,4-D (0.5 mg/L), kinetin (0.05 mg/L), and spectinomycin (100 mg/L; 3 d) followed by incubation on an ARM containing IAA (0.15 mg/L), phenyladenine (1.6 mg/L), and spectinomycin (100 mg/L; Motte et al., 2013). Seed was obtained by growing shoots on MS salt medium containing 3% (w/v) Suc and 0.8% (w/v) agar (A7921; Sigma), pH 5.8.

### Plant Materials and Growth Conditions

The Arabidopsis (Arabidopsis thaliana) Sav-0 (CS28725) and Col-0 homozygous acc2-1 knockout line (SALK_148966C) seeds were obtained from the Arabidopsis Biological Resource Center. The Col-0 seeds were obtained from Juan Dong (Rutgers University). The RLD and Ler seeds were purchased form Lehle Seeds.

For surface sterilization, seeds (25 mg) were treated with 1.7% (w/v) sodium hypochlorite (5× diluted 8.5% (w/v) commercial bleach) in a 1.5-mL Eppendorf tube for 15 min with occasional mixing (vortex). The bleach was removed by pipetting and washed three times with sterile distilled water. Seeds were germinated on 50 mL of ARM5 medium in deep petri dishes (20 mm high and 10 cm in diameter). The plates were illuminated for 8 h using cool-white fluorescent tubes (2,000 1×). The seeds germinated after 10 to 15 d of incubation at 24° C. To grow plants with larger leaves, seedlings were transferred individually to ARM5 plates (four plants per deep petri dish). The plates were illuminated for 8 h with cool-white fluorescent bulbs (2,000 lx) and incubated at 21° C. during the day and 18° C. during the night. One- to 2-cm-long. dark green leaves were harvested for bombardment after incubation for an additional 5 to 6 weeks.

### Plastid Transformation Vector

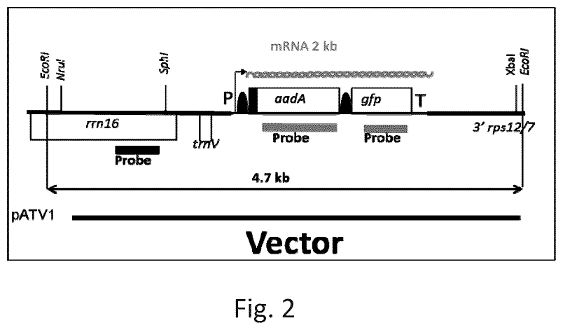

The plastid transformation vector pATV1 targets insertion in the inverted repeat region of the plastid genome upstream of the trnV gene (FIG. 2). The targeting region is a 4.5 kb NruI/XbaI fragment derived from the Arabidopsis thaliana ptDNA (GenBank Accession No. NC_000932). The fragment was cloned in the KpnI-SacI site of a pBSKS+ BlueScript vector, ligating the vector KpnI site to the plastid NruI site and vector SacI site to the plastid XbaI site. The vector carries a dicistronic operon, in which the first open reading frame (ORF) encodes the aadA spectinomycin resistance gene and the second ORF encodes a green fluorescence protein (GFP). The operon is expressed from the PrrnLatpB promoter, obtained by fusing the plastid rRNA operon promoter (Prrn) with the atpB plastid gene leader (LatpB), originally described in the pHK30 plasmid (Kuroda and Maliga, 2001). The dicistronic aadA-gfp marker gene was excised as an EcoRI-HindIII fragment and cloned in the HincII site of the targeting region. In the dicistronic construct, 14 N-terminal amino acids of the ATP synthase beta subunit are translationally fused with the AAD N-terminus, as in plasmid pHK30 (Kuroda and Maliga, 2001). The intergenic region encodes the cry9Aa2 gene leader (Chakrabarti et al., 2006), followed by the gfp coding region and the 3′-UTR of the plastid psbA gene (TpsbA) for the stabilization of the mRNA. The DNA sequence of the EcoRI-HindIII fragment encoding the aadA-gfp dicistronic operon in plasmid pMRR13 is shown below.

(SEQ ID NO: 1)
gagctcGCTCCCCCGCCGTCGTTCAATGAGAATGGATAAGAGGCTCGTGG
GATTGACGTGAGGGGGCAGGGATGGCTATATTTCTGGGAGAATTAACCGA
TCGACGTGCaAGCGGACATTTATTTTaAATTCGATAATTTTTGCAAAAAC
ATTTCGACATATTTATTTATTTTATTATTATGAGAATCAATCCTACTACT
TCTGGTTCTGGGGTTTCCACGgctactagcGAAGCGGTGATCGCCGAAGT
ATCGACTCAACTATCAGAGGTAGTTGGCGTCATCGAGCGCCATCTCGAAC
CGACGTTGCTGGCCGTACATTTGTACGGCTCCGCAGTGGATGGCGGCCTG
AAGCCACACAGTGATATTGATTTGCTGGTTACGGTGACCGTAAGGCTTGA
TGAAACAACGCGGCGAGCTTTGATCAACGACCTTTTGGAAACTTCGGCTT
CCCCTGGAGAGAGCGAGATTCTCCGCGCTGTAGAAGTCACCATTGTTGTG
CACGACGACATCATTCCGTGGCGTTATCCAGCTAAGCGCGAACTGCAATT
TGGAGAATGGCAGCGCAATGACATTCTTGCAGGTATCTTCGAGCCAGCCA
CGATCGACATTGATCTGGCTATCTTGCTGACAAAAGCAAGAGAACATAGC
GTTGCCTTGGTAGGTCCAGCGGCGGAGGAACTCTTTGATCCGGTTCCTGA
ACAGGATCTATTTGAGGCGCTAAATGAAACCTTAACGCTATGGAACTCGC
CGCCCGACTGGGCTGGCGATGAGCGAAATGTAGTGCTTACGTTGTCCCGC
ATTTGGTACAGCGCAGTAACCGGCAAAATCGCGCCGAAGGATGTCGCTGC
CGACTGGGCAATGGAGCGCCTGCCGGCCCAGTATCAGCCCGTCATACTTG
AAGCTAGACAGGCTTATCTTGGACAAGAAGAAGATCGCTTGGCCTCGCGC
GCAGATCAGTTGGAAGAATTTGTCCACTACGTGAAAGGCGAGATCACCAA
GGTAGTgGGCAAAgaaCAAAAACTCATTTCTGAAGAAGACTTGTAACTGC
AGATAACCCAAATAATGTTTTAAAATTTTAAAAATAATGTAGGAGGAAAA
ATTATGGCTAGCAGTAAAGGAGAAGAACTTTTCACTGGAGTTGTCCCAAT
TCTTGTTGAATTAGATGGTGATGTTAATGGGCACAAATTTTCTGTCAGTG
GAGAGGGTGAAGGTGATGCAACATACGGAAAACTTACCCTTAAATTTATT
TGCACTACTGGAAAACTACCTGTTCCtTGGCCAACACTTGTCACTACTTT
CTCTTATGGTGTTCAATGCTTTTCAAGATACCCAGATCATATGAAGCGGC
ACGACTTCTTCAAGAGCGCCATGCCTGAGGGATACGTGCAGGAGAGGACC
ATCTCTTTCAAGGACGACGGGAACTACAAGACACGTGCTGAAGTCAAGTT
TGAGGGAGACACCCTCGTCAACAGGATCGAGCTTAAGGGAATCGATTTCA
AGGAGGACGGAAACATCCTCGGCCACAAGTTGGAATACAACTACAACTCC
CACAACGTATACATCACGGCAGACAAACAAAAGAATGGAATCAAAGCTAA
CTTCAAAATTAGACACAACATTGAAGATGGAAGCGTTCAACTAGCAGACC
ATTATCAACAAAATACTCCAATTGGCGATGGCCCTGTCCTTTTACCAGAC
AACCATTACCTGTCCACACAATCTGCCCTTTCGAAAGATCCCAACGAAAA
GAGAGACCACATGGTCCTTCTTGAGTTTGTAACAGCTGCTGGGATTACAC
ATGGCATGGATGAACTATACAAATAAGctctagCTAGAGCgatcctggcc
tagtctataggaggttttgaaaagaaaggagcaataatcattttcttgtt
ctatcaagagggtgctattgctcctactattactattatttatttactag
tatatacttacatagacttttttgtttacattatagaaaaagaaggagag
gttattttcttgcatttattcatgGGGGATCAAAGCTT

### Transformation and Selection of Transplastomic Lines

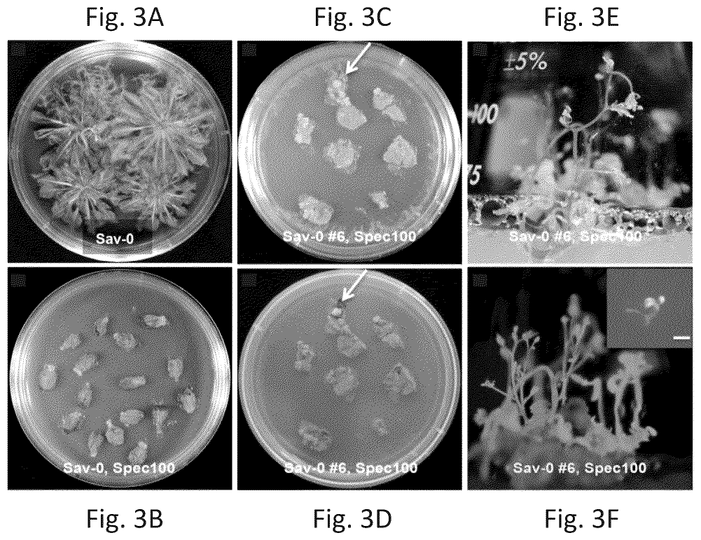

Plastid transformation in Arabidopsis was carried out using our 1998 protocol, as shown in FIG. 3A-3F (Sikdar et al., 1998). The leaves (10 to 20 mm) were harvested from aseptically grown plants and covered the surface of agar-solidified ARMI medium in a 10 cm petri dish. We used ˜100 leaves to cover the surface of the plate. The leaves were cultured for 4 days on ARMI medium, then bombarded with pATV1 vector DNA. Transforming DNA was coated on the surface of microscopic (0.6 um) gold particles, then introduced into chloroplasts by the biolistic process (1,100 psi) using a helium-driven PDS1000/He biolistic gun equipped with the Hepta-adaptor (Lutz et al., 2011). The plates were placed on the shelf at the lowest position for bombardment.

Following bombardment, the leaves were incubated for an additional 2 d on ARMI medium. After this time period, the leaves were stamped with a stack of 10 razor blades to create parallel incisions 1 mm apart. The stamped leaves were cut into smaller (1 cm2) pieces, transferred onto the same medium (ARMI) containing 100 mg/L spectinomycin, incubated at 28° C., and illuminated for 16 h with fluorescent tubes (CXL F025/741). After 8 to 10 d, the leaf strips were transferred onto selective ARMIIr medium containing 100 mg/L spectinomycin for the selection of spectinomycin-resistant clones. The leaf strips were transferred to a fresh selective ARMIIr medium every 2 weeks until putative transplastomic clones were identified as resistant green calli.

### Confocal Microscopy to Detect GFP in Plastids

Subcellular localization of GFP fluorescence was determined by a Leica TCS SP5II confocal microscope. To detect GFP and chlorophyll fluorescence, excitation wavelengths were at 488 nm and 568 nm, and the detection filters were set to 500-530 nm and 650-700 nm, respectively.

### DNA and RNA Gel-Blot Analyses

Total leaf DNA was prepared by the cetyltrimethylammonium bromide protocol (Tungsuchat-Huang and Maliga, 2012). DNA gel-blot analyses was carried out as described (Svab and Maliga, 1993). Total cellular DNA was digested with the EcoRI restriction enzyme. The DNA probe was the ApaI-SphI ptDNA fragment encoding the plastid rrn16 gene (FIG. 2).

Total cellular RNA was isolated from leaves frozen in liquid nitrogen using TRIzol (Ambion/Life Technologies) following the manufacturer's protocol. RNA gel-blot analyses were carried out as described (Kuroda and Maliga, 2001). The probes were as follows: for aadA, a 0.8-kb NcoI-XbaI fragment isolated from plasmid pHC1 (Carrer et al., 1991); and for gfp, a fragment amplified from the gfp coding region using primers gfp-forward p1 (5′-TTTTCTGTCAGTGGAGAGGGTG-3′) (SEQ ID NO: 2) and gfp-reverse p2 (5′-CCCAGCAGCTGTTACAAACT-3′ (SEQ ID NO: 3) (FIG. 2).

### Alignment of Homomeric ACCases

The alignment of homomeric ACCases in the Brassicaceae family was carried out with MultAlin software (Corpet, 1988).

### Accession Numbers

The DNA sequence of the pATV1 Arabidopsis plastid transformation vector was deposited in GenBank under accession number MF461355.

The following examples are provided to illustrate certain embodiments of the invention. They are not intended to limit the invention in any way.

### Example I

### Dicistronic pATV1 Vector for Identification of Transplastomic Events

The plastid transformation vector pATV1 targets insertion upstream of the trnV gene in the inverted repeat region of the plastid genome (FIG. 2). Vector pATV1 carries a dicistronic operon, in which the first open reading frame (ORF) encodes the aadA spectinomycin resistance gene and the second ORF encodes a green fluorescence protein (GFP) (FIG. 2). Polycistronic mRNAs are not translated on the eukaryotic-type 80S ribosomes in the cytoplasm, thus accumulation of GFP in chloroplasts in spectinomycin-resistant clones indicates plastid transformation.

### Plastid Transformation and Identification of Transplastomic Events

Plastid transformation was carried out in the Col-0 (Columbia) accession and the Columbia ACC2 T-DNA insertion line acc2-1 (SALK_148966C) shown to be sensitive to spectinomycin in the Parker at el. study (Parker et al., 2014). We also evaluated plastid transformation efficiency in the Sav-0 (Slavice) accession that was the most sensitive to spectinomycin in the study (Parker et al., 2014). The Sav-0 ACC2 gene carries 15 missense mutations, but one variant alone (G135E) that alters a conserved residue immediately preceding the biotin carboxylase domain appears to be responsible for the hypersensitive phenotype (Parker et al., 2016). Plants were grown aseptically on ARM5 medium (FIG. 3A); leaves for plastid transformation were harvested from sterile plants and placed on ARMI media. The leaf tissue was bombarded with gold particles coated with vector DNA. After two days, the leaves were stamped with a stack of razor blades to create a series of parallel incisions 1 mm apart. The mechanical wounds are essential to induce uniform callus formation in the leaf blades. The stamped leaves were transferred onto the same medium (ARMI) containing spectinomycin (100 mg/l; FIG. 3B) to facilitate preferential replication of plastids containing transformed ptDNA copies. The ARMI medium induces division of the leaf cells and formation of colorless, embryogenic callus. After 7-10 days of selection on ARMI medium, spectinomycin selection was continued on the ARMIIr medium, which induces greening. Since spectinomycin prevents greening of wild-type cells, only spectinomcyin-resistant cells formed green calli. Visible green cell clusters appeared within 21 to 40 days on the selective ARMIIr medium (FIG. 3C). Illumination of plates with UV light revealed intense fluorescence of GFP in the green calli (FIG. 3D).

In the wild-type Col-0 sample (four bombarded plates), no transplastomic event was found. We obtained eight events on five bombarded plates using leaf tissue in the acc2-1 mutant background and four events in four bombarded plates in the Sav-0 accession (Table 1). This transformation efficiency is comparable to the transformation efficiency obtained with current protocols in tobacco: four to five transplastomic events per bombardment (Maliga and Tungsuchat-Huang, 2014).

This is a significant advance, as high-frequency plastid transformation in Arabidopsis has been pursued since the publication of the original report (Sikdar et al., 1998) but has been largely unsuccessful. For example, bombardment of 26 plates of RLD and five plates of Landsberg erecta (Ler) leaf tissue did not yield a transplastomic event (Table 1). In contrast, nine bombardments of leaves with the acc2 null background yielded 12 transplastomic clones. Even though the technology improved significantly since 1998, no transplastomic clones were obtained until ACC2-defective leaf tissue was used for bombardments (Table 1), providing overwhelming support for the absence of ACC2 activity being critical for high-frequency plastid transformation in Arabidopsis.

TABLE 1
Identification of transplastomic events in Arabidopsis
Au, Gold particles; Hepta, using the biolistic gun Hepta adaptor instead of a single flying disk; Tu, tungsten particles.

| | | | | | | | No. of | |
|---|---|---|---|---|---|---|---|---|
| | Left/Right | | | | | | Trans- | |
| | Arm | | | | | No. of | plastomic | |
| Plasmid | kb | Marker Gene | Accession | Tissue | Gun | Plates | Events | Reference |
| pGS31A | 1.1/0.9 | Prrn:LrbcL:aadA:TpsbA | RLD | Leaf | Single, Tu/1 μm | 201 | 2 | Sikdar et al. (1998) |
| pAAK176 | 1.7/0.8 | Prrn:LrbcL:aadA:TpsbA | RLD | Leaf | Hepta, Au/0.6 μm | 10 | 0 | Reported here |
| | | | Ler | Leaf | Hepta, Au/0.6 μm | 4 | 0 | Reported here |
| pTT626 | 1.7/0.8 | Prrn:Lcry9:aadA-gfp:TpsbA | RLD | Leaf | Hepta, Au/0.6 μm | 14 | 0 | Reported here |
| pATV1 | 1.7/0.8 | PrrnLatpB:aadA:Lcry9:gfp:TpsbA | RLD | Leaf | Hepta, Au/1 μm | 2 | 0 | Reported here |
| | | | Ler | Leaf | Hepta, Au/1 μm | 1 | 0 | Reported here |
| | | | Col-0 | Leaf | Hepta, Au/0.6 μm | 4 | 0 | Reported here |
| | | | Col-0 acc2-1 | Leaf | Hepta, Au/0.6 μm | 5 | 8 | Reported here |
| | | | Sav-0 | Leaf | Hepta, Au/0.6 μm | 4 | 4 | Reported here |

Confocal Microscopy to Confirm Transplastomic events

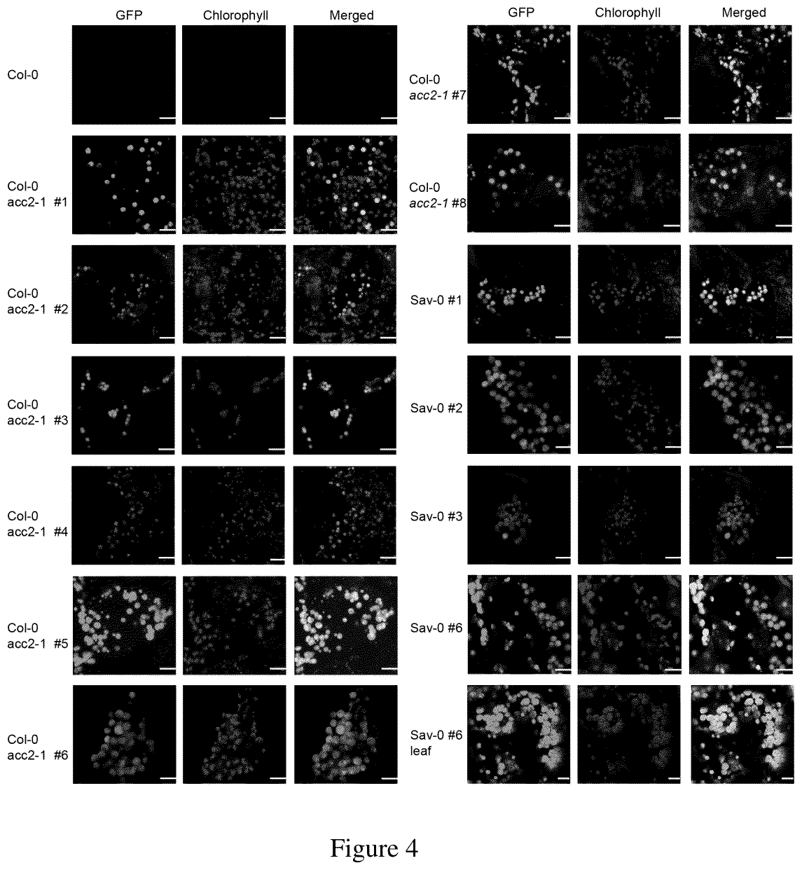

Because GFP is encoded in the second ORF, GFP accumulation is expected only if the mRNA is translated in plastids on the prokaryotic type 60S ribosomes known to translate transgenic polycistronic mRNAs. Examples are the plastid psbE operon (Carrillo et al., 1986; Willey and Gray, 1989), the psaA/B transcript (Meng et al., 1988) and petA, which is not cleaved off the upstream ycf10 gene (Willey and Gray, 1990). Translation of polycistronic mRNAs created by operon extension has also been demonstrated (Staub and Maliga, 1995). Thus, GFP accumulation was anticipated only if the gfp gene is expressed in chloroplasts. The putative transplastomic lines identified as green cell clusters have subsequently been confirmed as transplastomic events by detecting localization of GFP to plastids by confocal microscopy. Overlay of the GFP and chlorophyll channels indicates that the clones are heteroplastomic, carrying transformed and wild type plastids in the same cells. A good example for mixed plastids is shown in the overlay of GFP and chlorophyll channels in Col-0 acc2-1#3 in FIG. 4. The chloroplasts were not well developed in most tissue culture cells. Chlorophyll was detected in only a localized region of plastids in line with thylakoid biogenesis initiating from a localized center (Schottkowski et al., 2012). Good examples are overlays of Col-0 acc2-1#5 and Sav-0 #1 in FIG. 4.

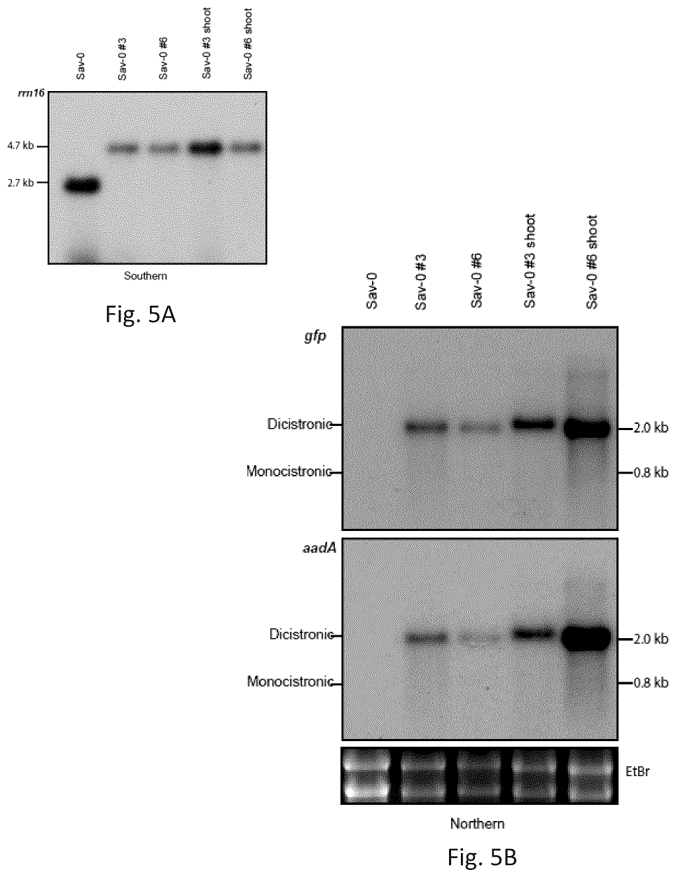

The heteroplastomic state detected in the cells of the green clusters was not maintained, and eventually, wild-type plastids (ptDNA) disappeared in the callus cells after continued cultivation on selective media. The homoplastomic state is confirmed by the uniform accumulation of GFP in the leaves of a Sav-0 #6 plant shown in FIG. 4 and by DNA gel-blot analyses of calli shown in FIG. 5B.

### Regeneration of Transplastomic Sav-0 Plants and Transmission of GFP to Seed Progeny

After the bombardment of Col-0 and Sav-0 leaves, the selection of transplastomic events was carried out according to the published RLD protocol (Sikdar et al., 1998). However, when the transplastomic clones were transferred to the RLD shoot induction medium, the calli did not proliferate. Therefore, we transferred the transplastomic calli to media that were used successfully to regenerate plants from other accessions. We found that the two-step regeneration protocol described for shoot induction in the C24 background (Motte et al., 2013) triggered shoot regeneration in two surviving Sav-0 calli. Calli of Sav-O transplastomic lines #3 and #6 were briefly (3 d) exposed to callus induction medium containing 0.5 mg/L 2,4-dichlorophenoxyactetic acid (2,4-D) and 0.05 mg/L kinetin and then transferred to a shoot regeneration medium containing 0.15 mg/L indole acetic acid (IAA) and 1.6 mg/L phenyladenine. Phenyladenine is a potent compound for shoot regeneration through the inhibition of cytokinin oxidase/dehydrogenase activity (Motte et al., 2013). Shoots from the calli developed in 45 to 60 days and flowered and formed siliques in sterile culture (FIG. 3E). The plants glowed intensely when illuminated with UV light, indicating high-level GFP accumulation

(FIG. 3F-3G). Confocal microscopy suggests uniform transformation of plastid genomes in the leaves of Sav-0 #6 plants (FIG. 4) and was confirmed by molecular analyses (FIG. 5B).

The transplastomic shoots were transferred to larger 500-mL Erlenmeyer flasks containing ARM for seed set, where they continued to grow.

### Molecular Analysis of Transplastomic Arabidopsis Clones

DNA and RNA gel blot analyses was carried out on the callus and shoots of the two Sav-0 transplastomic lines #3 and #6. Wild-type plastids present in the cells of the green clusters were gradually lost by the time DNA gel-blot analyses were carried out, confirming uniform transformation of the plastid genomes in both calli and shoots (FIG. 5A). RNA gel blot analyses indicated the presence of a 2-kb dicistronic transcript detected by both the aadA and gfp probes (FIG. 5B).

### Discussion

Development of successful plastid transformation protocols takes multiple years, explaining the relative paucity of crops in which plastid transformation is routine (Maliga and Bock, 2011; Maliga, 2012; Bock, 2015). The expectation is to obtain transplastomic plants, which carry and transmit to the seed progeny a uniformly transformed plastid genome population. The time required to obtain a flowering transplastomic plants from seed takes about 5 to 6 months, as outlined in Table 2. This time frame can be broken up into discrete steps, each of which represents a milestone in developing a complete system. We report here a significant break-through: high frequency transformation of the Arabidopsis plastid genome in spectinomycin sensitive accessions and a marker system that enables rapid identification of transplastomic events by selective expression of a GFP gene in plastids. This step is a major advance towards developing a complete system of plastid engineering in Arabidopsis.

TABLE 2
An overview of the protocol for the construction
of a transplastomic Arabidopsis Sav-0 plants.

| | | | TIME |
|---|---|---|---|
| | OBJECTIVE | CULTURE MEDIUM | (No. of transfers) |
| Step 1 | Seed germination | ARM5 Medium | 14 days |
| Step 2 | Grow sterile plants | ARM5 medium | 42 days |
| Step 3 | Leaf callus, non | ARMI medium | 4 days |
| | selective | | |
| Step 4 | Leaf bombardment | ARMI medium | |
| Step 5 | Leaf callus, non | ARMI medium | 2 days |
| | selective | | |
| Step 6 | Leaf callus, | ARMI medium + | 14 days |
| | selective | Spectinomycin | |
| | | (100 mg/L) | |
| Step 7 | Leaf callus, | ARMIIr + | 21 days (2x) |
| | greening | Spectinomycin | |
| | | (100 mg/L) | |
| Step 8 | Shoot induction | ARM medium + 2,4-D | 3 days |
| | | (0.5 mg/L), kinetin | |
| | | (0.05 mg/L), | |
| | | Spectinomycin | |
| | | (100 mg/L) | |
| Step 9 | Shoot regeneration | ARM medium + IAA | 45-60 days |
| | | (0.15 mg/L), Phe-Ade | |
| | | (1.6 mg/L), | |
| | | Spectinomycin | |
| | | (100 mg/L) | |

Time to flowering plants: 145-160 days (~5 months)

### Development of a Plastid Transformation Protocol in Arabidopsis

The steps of a complete system of plastid engineering in Arabidopsis consist of: (a) obtaining or generating sterile acc2 defective plants to provide a leaf source for transformation; (b) delivering DNA to plastids; (c) recovering transplastomic events; (d) regenerating shoots from transplastomic callus and (e) obtaining seed from the shoots.

We report here approximately 100-fold enhanced plastid transformation efficiency per bombardment in the acc2 null background: eight events in five bombarded samples in the Col-0 acc2-1 line and four events in four bombarded samples in the Sav-0 background. The increase from one event per approximately 100 bombardments to one event per one bombardment is due in part to technological advances. However, the lack of success with the latest technology in a large number of bombarded samples (Table 1) provides overwhelming evidence that the key to success was the choice of Arabidopsis lines lacking ACC2 activity.

Identification of transplastomic events in the RLD ecotype took 5 to 12 weeks in 1998 (Sikdar et al., 1998). The use of spectinomycin-sensitive acc2-knockout lines and the pATV1 dicistronic operon vector shortened the time period for identification of transplatomic events to 3 to 5 weeks. The use of the acc2 knockout lines shortened scoring because the proliferation of non-transformed cells growth was efficiently inhibited by spectinomycin, enabling identification of the spectinomycin-resistant green cell clusters. Spectinomycin resistance may be due to the integration of aadA in the plastid genome, and fortuitous expression from an upstream promoter or spontaneous mutations in the rrn16 gene (Svab and Maliga, 1993). GFP, encoded in the second ORF, is expressed only in chloroplasts, enabling the rapid identification of transplastomic clones in a small number of heteroplastomic cells by confocal microscopy.

Once transplastomic clones are identified, the next major step is plant regeneration. There is diversity for shoot regeneration potential in Arabidopsis accessions. Col-0 is well known for its recalcitrance to shoot regeneration from cultured cells. Therefore, no attempt was made to regenerate shoots from the Col-0 transplastomic callus tissue. There is no information about the tissue culture properties of the Sav-0 accession. Our first attempts at Sav-0 shoot regeneration from the transplastomic clones proved successful, yielding flowering shoots in culture (FIG. 3E). However, the seeds, with one exception, failed to germinate. Shoot regeneration protocols have been worked out from root (Marton and Browse, 1991) and leaf explants (Lutz et al., 2015) of the RLD ecotype; and from protoplasts (Chupeau et al., 2013), leaf explants (Zhao et al., 2014) and inflorescence stem explants (Zhao et al., 2013) of the Wassilewskya (Ws) ecotype. Thus, a routine protocol for plastid transformation in Arabidopsis can be obtained by the refinement of leaf regeneration protocol in the Sav-0 ecotype, or by developing ACC2 knockout mutations in the RLD (Marton and Browse, 1991) or Wassilewskya (Ws) (Chupeau et al., 2013; Zhao et al., 2014) nuclear backgrounds. Alternatively, the Col-0 acc2-1 can be transformed with the steroid-inducible BABYBOOM gene to facilitate plant regeneration from transplastomic events (Lutz et al., 2015).

Seed from transplastomic tobacco is obtained by rooting shoots in tissue culture, then transferring the rooted cuttings to a greenhouse. Arabidopsis shoots obtained in tissue culture are notoriously difficult to root. Rather than making an effort to root the plants in culture and transfer them to the greenhouse, we obtained seed from plants in sterile culture, a two-three month process (Lutz et al., 2015).

### Early Identification of Plastid Transformants

The dicistronic marker system is a developer's tool that enables carly scoring, but severely burdens the developing plants due to the high level of AAD and GFP expression, ˜7% and ˜15% of total soluble cellular protein (TSP) in tobacco, respectively (unpublished). High-levels of AAD are not necessary to obtain transplastomic plants. We have found that a mutation in the promoter of the aadA gene reduced accumulation of AAD gene product below 1% without impact on the frequency of transplastomic events by spectinomycin selection (Sinagawa-Garcia et al., 2009). Therefore, the new Arabidopsis vectors expressing low levels of AAD described herein can be used to advantage as lowered expression levels of AAD do not compromise plant growth.

### Plastid Transformation in Arabidopsis Provides Template for Recalcitrant Crops

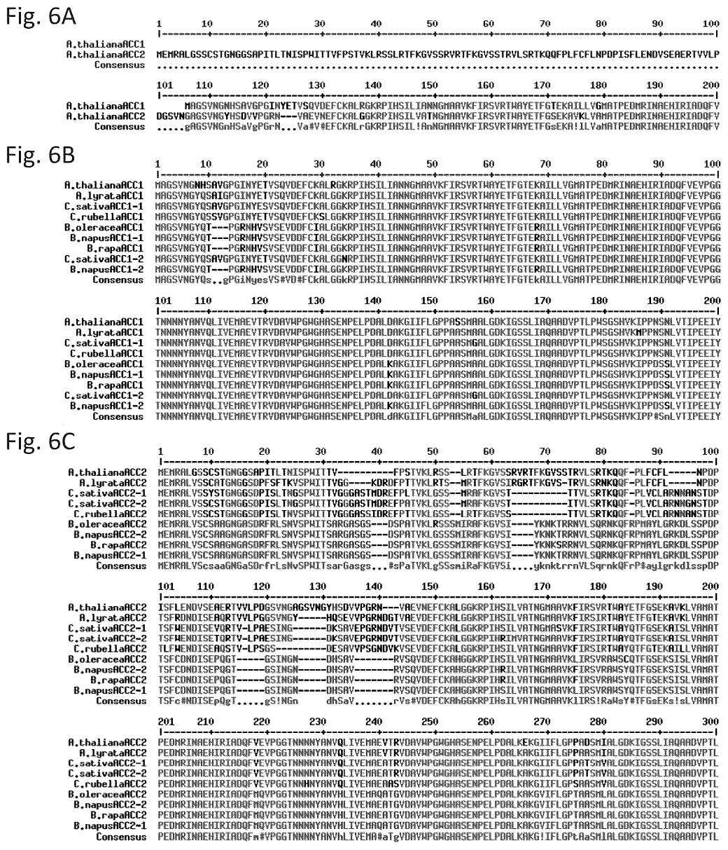

The recognition that the duplicated ACCase in Arabidopsis is an impediment to plastid transformation provides the guidance necessary for implementation of plastid transformation in all Arabidopsis accessions and in crops having a plastid-encoded accD gene and a plastid-targeted ACC2 enzyme. The Arabidopsis thaliana ACC2 enzyme has an N-terminal extension compared to ACC1 (FIG. 6A). The N-terminal extension is a plastid targeting sequence shown by subcellular localization of a GFP fusion protein (Babiychuk et al., 2011). The ACC1 and ACC2 genes are present in most Brassicaceae species, including Arabidopsis lyrata, Camelina sativa, Camelina rubella, Brassica oleracea, Brassica napus and Brassica rapa. The homomeric ACC2 enzyme in these species has an N-terminal extension compared to ACC1 (FIG. 6B and 6C). Thus, a targeted mutation in the N-terminal extension can selectively inactivate the ACC2 variant to create a spectinomycin hypersensitive variant similar to the Col-0 acc2-1 deletion derivative (Parker et al., 2014).

Crops recalcitrant to plastid transformation such as cotton (Gossypium raimondii), soybean (Glycine max) and alfalfa (Medicago truncatula) have a plastid accD gene and multiple homomeric nuclear ACC genes. Indeed, this method should prove effective in those plants having comparable ACC2 with an N-terminal extension. Moreover, further experimentation could be performed to determine how deletion of one or more of the homomeric ACCase genes enhances recovery of transplastomic events.

Mutations in genes other than ACC2 also made Arabidopsis sensitive to spectinomycin.

The TIC20-IV gene, which is required for the import of proteins through the inner chloroplast membrane, appears to limit the import of ACC2 enzyme (Parker et al., 2014). Dicot plastid genomes have several essential genes, including accD, clpP, Ycf1 and Ycf2 (Scharff and Bock, 2014). Apparently, in photoheterotrophic cultures where sucrose in the medium eliminates the need for photosynthesis, only translation of the accD mRNA, hence fatty acid biosynthesis, is required to sustain plant life.

### CONCLUSION

Boost of plastid transformation efficiency using ACC2 knockout lines in commercial species of Brassicaceae has obvious economic benefits. Genomic resources make Arabidopsis the favored model to study basic biological processes, and to explore new biotechnological applications (Weigel and Mott, 2009; Koornneef and Meinke, 2010; Stitt et al., 2010; Wallis and Browse, 2010). The exception is photosynthesis research and chloroplast biotechnology that utilizes tobacco (Nicotiana tabacum) because engineering of the plastid genome encoding key components of the photosynthetic machinery is routine in only this species (Hanson et al., 2016; Sharwood et al., 2016). If plastid transformation would be available in Arabidopsis, this research would be carried out in this model organism, in which a large mutant collection is available in virtually any nuclear gene contributing to photosynthesis. Recognizing the importance of plastid translation during selection of transplastomic events has identified a bottleneck of plastid transformation in Arabidopsis. High frequency plastid transformation in Arabidopsis thaliana will open up the unique resources of this model species to advance our understanding of plastid function and new biotechnological applications.

### Example II

### Deletion of ACC2 Genes in Brassicaceae Crops to Create Suitable Recipients for Plastid Transformation

As discussed above in Example I, crops in the Brassicaceae family encode homologs of the Arabidopsis ACC2 gene, characterized by an N-terminal extension as compared to ACC1. Manual inspection of the N-terminal region of ACC2 genes led to the identification of >20 suitable guide RNAs (see Table 3). The potential gRNAs targeting both stands (5′ to 3′ and 3′ to 5′) are identified as NNNNNNNNNNNNNNNNNNNN NGG sequence (20N+NGG, N=A/G/C/T) (SEQ ID NO: 4), where the only limitation is the presence of a GG sequence (Mali et al., 2013). More relaxed rules for sgRNA design can be used in plants, such as G(N)₁₉₋₂₂ for the U6 promoter and A(N)₁₉₋₂₂ for the U3 promoter and the 1^{st} nucleotide does not have to match the genomic sequence (Belhaj et al., 2013).

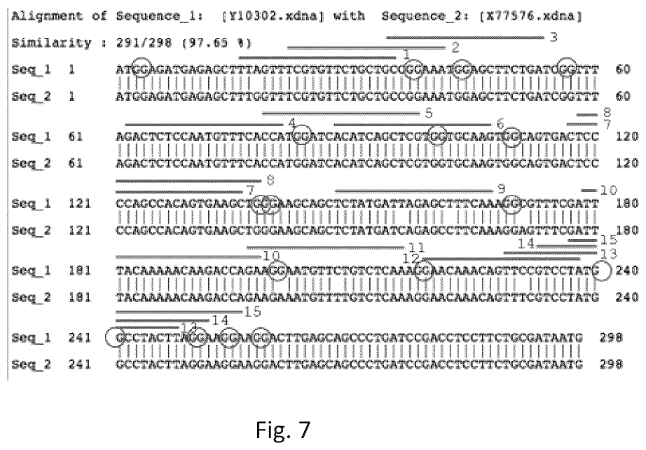

Brassica napus L. (AACC, 2n=4x=38) is an amphidoploid species originating from spontancous hybridization of Brassica rapa (AA, 2n=2x-20) and Brassica oleracea (CC, 2n=2x=18) (Song and Osborn, 1992; Howell et al., 2008). The Brassica napus genome encodes two ACC1 genes (Locus 106413885; Locus 106418889) and two ACC2 genes (GenBank accession numbers X77576, Y10302) (Schulte et al., 1997). Simultaneous mutation of two genomic sequences can be executed efficiently using CRISPR/Cas9, as described in the literature. A noteworthy example is simultaneous inactivation of 62 copies of a porcine endogenous retrovirus in pigs (Yang et al., 2015). Additionally, non-segregating seed progeny due to mutations in both genomic copies in the first generation of Arabidopsis and tomato plants (Feng et al., 2014) (Brooks et al., 2014). The alignment of 298 N-terminal nucleotides of the Brassica napus ACC2 genes reveals 7 mismatches. Still, 9 of the 15 potential forward sgRNAs are useful for simultaneously inducing mutations in both ACC2 gene copies (FIG. 7, Table 3).

To achieve targeted deletion in the ACC2 N-terminal region, the gRNAs are cloned into the CRISPR/Cas vector and introduced into different crops using a nuclear transformation system appropriate for the target species. For example, Camelina sativa plants will be transformed by the flower dip protocol (Liu et al., 2012). In the case of Brassica, introduction of the CRISPR/Cas vector system can be achieved using Agrobacterium-mediated transformation of hypocotyls (Cardoza and Stewart, 2003, 2006) or flower dip transformation (Tan et al., 2011; Verma et al., 2008) as described below.

### Agrobacterium-Mediated Transformation of Hypocotyl Segments

Brassica napus L. cv. Westar is transformed with an Agrobacterium binary vector carrying kanamycin resistance as a plant marker. Seeds are surface-sterilized with 10% sodium hypochlorite with 0.1% Tween for 5 minutes, followed by a 1-min rinse with 95% ethanol and washing the seed 5x with sterile distilled water. The seeds are germinated in sterile culture on MS basal medium (Murashige and Skoog, 1962) containing 20 g/l sucrose and solidified with 2 g/l Gelrite. Hypocotyls for transformation are excised from 8 to 10-day-old seedlings and 1-cm pieces preconditioned for 48 h on MS medium supplemented with 1 mg/l 2,4-D (2,4-dichlorophenoxy acetic acid) and 30 g/l sucrose, solidified with 2 g/l Gelrite. The preconditioned hypocotyl segments were then inoculated with Agrobacterium grown overnight to an OD600=0.8 in liquid LB medium. The Agrobacterium cells are pelleted by centrifugation and re-suspended in liquid callus induction medium with 0.05 mM acetosyringone to induce T-DNA transfer.

Co-cultivation with Agrobacterium is performed for 48 h on MS medium with 1 mg/l 2,4-D. Following co-cultivation, the explants are transferred to the same medium with 400 mg/l timentin and 200 mg/l kanamycin to select for transformed cells. After 2 weeks, the explants are transferred to MS medium to promote organogenesis containing 4 mg/l BAP (6-benzylaminopurine), 2 mg/l zeatin, 5 mg/l silver nitrate, 400 mg/l timentin, 200 mg/l kanamycin and 30 g/l sucrose, solidified with 2 g/l Gelrite. After an additional 2 weeks, the tissue is transferred to MS medium containing 3 mg/l BAP, 2 mg/l zeatin, the same antibiotics, 30 g/l sucrose and 2 g/l Gelrite for shoot development. To encourage shoot elongation, the shoots are transferred to MS medium with 0.05 mg/l BAP, 30 g/l sucrose, antibiotics as above, solidified with 3 g/l Gelrite. The elongated shoots are rooted on a medium containing half-strength MS salts, 10 mg/l sucrose, 3 g/l Gelrite, 5 mg/l IBA, and 400 mg/l timentin and 200 mg/l kanamycin. The cultures are incubated at 25±2° C., 16/8-h (light/dark) photoperiod. The rooted shoots are transferred to soil and grown at 20° ° C.20, 16/8 h (light/dark) photoperiod. To prevent desiccation, the plants are initially covered with a plastic dome.

### Floral Dip Transformation in Brassica ssp. to Generate ACC2 Defective Plants

For Agrobacterium-mediated floral dip transformation of Brassica napus, for example cv. Westar, more recent protocols that do not require vacuum infiltration are preferred. Verma et al. (2008) and Tan et al. (2011) report such protocols. Verma et al. (2008) recommends growing up the Agrobacterium strain in a selective medium, harvesting the cells by centrifugation and then re-suspending them in transformation medium comprising half MS salts, 5% sucrose, 0.05% Silwet L-77 to obtain the desired density (OD600=0.8 to 2.0). Plants are inoculated by submerging inflorescences in the bacterial suspension for one minute and then the inflorescences are wrapped with Saran wrap for 24 h to maintain the humidity. Seeds are collected at maturity and germinated on a selective medium to identify T1 seedlings by the expression plant marker encoded in the T-DNA.

A variant of this protocol is described by (Tan et al., 2011). Agrobacterium cultures carrying a target construct are collected by centrifugation and then resuspended in a solution containing 0.53 MS salts, 3% Sucrose, 0.1% Silwet L-77, 2 mg/L 6-benzyladenine, and 8 mg/L acetosyringone. The inflorescence of flowering plants is dipped into a beaker containing the Agrobacterium culture for 1 to 2 min with gentle agitation, and the treated inflorescence is wrapped with Saran wrap to keep the flowers most. The plants are treated three times at two day intervals, then the plants are allowed to grow to maturation. Seeds harvested from the transformed plants were surface sterilized and sown on the MS medium containing the plant marker encoded in the T-DNA. If kanamycin resistance is the plant marker, 200 mg/L kanamycin is used to screen for putative transformants. The putative transformants are identified upon the initiation of the first pair of green true leaves. Additional protocols for floral dip transformation are listed in Table 3 below.

TABLE 3

| Species | Reference |
|---|---|
| Brassica rapa L. ssp chinensis | (Qing et al., 2000) |
| Brassica campestris | (Liu et al., 1998) |
| L. ssp chinensis | |
| B. napus | (Wang et al., 2003; |
| | Wang et al., 2005; |
| | Tan et al., 2011) |
| B. napus, B. carinata, | (Verma et al., 2008) |
| high freq. | |
| Camelina sativa | (Lu and Kang, 2008) |

There are several Agrobacterium vector systems that have been described for CRISPR/Cas mutagenesis in plants (Belhaj et al., 2013; Li et al., 2014). We prefer the system described by Mao et al. for its simplicity (Mao et al., 2013). When a population of homozygous ACC2 knockout or biallelic mutant population is obtained, the seeds will be germinated on spectinomycin medium to identify the ACC2 defective plants by spectinomycin sensitivity (Parker et al., 2014). The type of knockout mutation will be verified by sequencing the target region and the progeny will be used as recipient in chloroplast transformation experiments. Brassica juncea is also an oilseed crop. The genomics of this crop is relatively undeveloped. However, guide RNAs to knockout the ACC2 gene can be designed using the principles outlined for the other Brassicaceae species as described herein above.

TABLE 3
Identification of guide RNAs in the N-terminal extension
of the ACC2 gene in Brassicaceae species. Shown are
the N-terminal protein sequence, the corresponding
cDNA sequence and the potential gRNAs.

>A. thaliana ACC2 (At1g36180)
>MEMRALGSSCSTGNGGSAPITLTNISPWITTVFPSTVKLRSSLRTFKGVSSRVRTFKGVS
STRVLSRTKQQFPLFCFLNPDPISFLENDVSEAERTVVLP (SEQ ID NO: 5)
>ATGGAGATGAGAGCTTTGGGTTCTTCGTGTTCTACTGGTAATGGAGGTTCTGCTCC
GATTACCCTCACGAATATATCTCCATGGATCACAACAGTTTTTCCGTCGACAGTGAA
GCTGAGAAGTAGTTTGAGAACCTTCAAAGGAGTTTCGTCAAGAGTGAGAACCTTTA
AAGGAGTTTCTTCGACAAGAGTTTTGTCTCGGACCAAACAACAGTTTCCTCTGTTTTG
TTTCCTAAACCCTGATCCGATCTCCTTCTTGGAAAATGATGTATCTGAAGCTGAAAG
GACAGTAGTTTTACCG (SEQ ID NO: 6)
For potential gRNAs, see SEQ ID NO. 239-254 below.
>A. lyrata ACC2 (XM_GG2891167.1)
>MEMRALVSSCATGNGGSDPFSFTKVSPWITTVGGKDRDFPTTVKLRTSMRTFKGVSIR
GRTFKGVSTRVLSRNKQQFPLFCFLNPDPTSFRDNDISEAQR (SEQ ID NO: 7)
>5′-3′
ATGGAGATGAGAGCTTTGGTTTCTTCGTGTGCTACCGGTAATGGAGGTTCTGATCCG
TTTAGCTTCACGAAAGTTTCTCCATGGATCACAACAGTTGGTGGTAAGGACAGAGAT
TTTCCAACGACAGTGAAGCTAAGAACTAGTATGAGAACCTTTAAAGGAGTTTCTATA
AGAGGGAGAACCTTTAAAGGAGTTTCGACAAGAGTTTTGTCTCGGAACAAACAACA
GTTTCCTCTGTTTTGTTTCCTAAACCCTGATCCGACCTCCTTCCGGGATAATGATATA
TCTGAAGCTCAAAGG (SEQ ID NO: 8)
TTGGTTTCTTCGTGTGCTAC CGG (SEQ ID NO: 9)
TCTTCGTGTGCTACCGGTAA TGG (SEQ ID NO: 10)
TCGTGTGCTACCGGTAATGG AGG (SEQ ID NO: 11)
GCTTCACGAAAGTTTCTCCA TGG (SEQ ID NO: 12)
TCTCCATGGATCACAACAGT TGG (SEQ ID NO: 13)
CCATGGATCACAACAGTTGG TGG (SEQ ID NO: 14)
GATCACAACAGTTGGTGGTA AGG (SEQ ID NO: 15)
ACTAGTATGAGAACCTTTAA AGG (SEQ ID NO: 16)
TTTAAAGGAGTTTCTATAAG AGG (SEQ ID NO: 17)
ATAAGAGGGAGAACCTTTAA AGG (SEQ ID NO: 18)
TTTCGACAAGAGTTTTGTCT CGG (SEQ ID NO: 19)
ACCCTGATCCGACCTCCTTC CGG (SEQ ID NO: 20)
ATGATATATCTGAAGCTCAA AGG (SEQ ID NO: 21)
>3′-5′
CCTTTGAGCTTCAGATATATCATTATCCCGGAAGGAGGTCGGATCAGGGTTTAGGAA
ACAAAACAGAGGAAACTGTTGTTTGTTCCGAGACAAAACTCTTGTCGAAACTCCTTT
AAAGGTTCTCCCTCTTATAGAAACTCCTTTAAAGGTTCTCATACTAGTTCTTAGCTTC
ACTGTCGTTGGAAAATCTCTGTCCTTACCACCAACTGTTGTGATCCATGGAGAAACT
TTCGTGAAGCTAAACGGATCAGAACCTCCATTACCGGTAGCACACGAAGAAACCAA
AGCTCTCATCTCCAT (SEQ ID NO: 22)
CTTCAGATATATCATTATCC CGG (SEQ ID NO: 23)
AGATATATCATTATCCCGGA AGG (SEQ ID NO: 24)
TATATCATTATCCCGGAAGG AGG (SEQ ID NO: 25)
TCATTATCCCGGAAGGAGGT CGG (SEQ ID NO: 26)
TCCCGGAAGGAGGTCGGATC AGG (SEQ ID NO: 27)
CCCGGAAGGAGGTCGGATCA GGG (SEQ ID NO: 28)
AGGAGGTCGGATCAGGGTTT AGG (SEQ ID NO: 29)
GGGTTTAGGAAACAAAACAG AGG (SEQ ID NO: 30)
TCTTGTCGAAACTCCTTTAA AGG (SEQ ID NO: 31)
TCTTATAGAAACTCCTTTAA AGG (SEQ ID NO: 32)
GTTCTTAGCTTCACTGTCGT TGG (SEQ ID NO: 33)
CCACCAACTGTTGTGATCCA TGG (SEQ ID NO: 34)
GAAACTTTCGTGAAGCTAAA CGG (SEQ ID NO: 35)
CGGATCAGAACCTCCATTAC CGG (SEQ ID NO: 36)
>C. sativa ACC2-1(LOC104777495)
>MEMRALVSSYSTGNGGSDPISLTNGSPWITTVGGGASTMDREFPLTVKLGSSMRAFKG
VSTTTVLSRTKQQFPLVCLARNNANSTDPTSFWENDISEVQR (SEQ ID NO: 37)
>5′-3′
ATGGAGATGAGAGCTTTGGTTTCTTCGTATTCTACCGGTAATGGAGGTTCTGATCCG
ATCAGCCTCACGAATGGTTCTCCATGGATCACAACAGTTGGTGGTGGTGCAAGTACC
ATGGACAGAGAGTTTCCATTGACTGTGAAGCTGGGAAGTAGTATGAGAGCCTTCAA
AGGAGTAAGCACAACAACAGTTTTGTCTCGGACCAAACAACAGTTTCCTCTGGTATG
CTTAGCAAGAAACAATGCGAACAGCACTGATCCGACCTCGTTCTGGGAGAATGATA
TATCTGAAGTTCAAAGG (SEQ ID NO: 38)
TTGGTTTCTTCGTATTCTAC CGG (SEQ ID NO: 39)
TCTTCGTATTCTACCGGTAA TGG (SEQ ID NO: 40)
TCGTATTCTACCGGTAATGG AGG (SEQ ID NO: 41)
GATCCGATCAGCCTCACGAA TGG (SEQ ID NO: 42)
GCCTCACGAATGGTTCTCCA TGG (SEQ ID NO: 43)
TCTCCATGGATCACAACAGT TGG (SEQ ID NO: 44)
CCATGGATCACAACAGTTGG TGG (SEQ ID NO: 45)
TGGATCACAACAGTTGGTGG TGG (SEQ ID NO: 46)
TGGTGGTGGTGCAAGTACCA TGG (SEQ ID NO: 47)
GTTTCCATTGACTGTGAAGC TGG (SEQ ID NO: 48)
AGTAGTATGAGAGCCTTCAA AGG (SEQ ID NO: 49)
GCACAACAACAGTTTTGTCT CGG (SEQ ID NO: 50)
GACCAAACAACAGTTTCCTC TGG (SEQ ID NO: 51)
GCACTGATCCGACCTCGTTC TGG (SEQ ID NO: 52)
ATGATATATCTGAAGTTCAA AGG (SEQ ID NO: 53)
>3′-5′
CCTTTGAACTTCAGATATATCATTCTCCCAGAACGAGGTCGGATCAGTGCTGTTCGC
ATTGTTTCTTGCTAAGCATACCAGAGGAAACTGTTGTTTGGTCCGAGACAAAACTGT
TGTTGTGCTTACTCCTTTGAAGGCTCTCATACTACTTCCCAGCTTCACAGTCAATGGA
AACTCTCTGTCCATGGTACTTGCACCACCACCAACTGTTGTGATCCATGGAGAACCA
TTCGTGAGGCTGATCGGATCAGAACCTCCATTACCGGTAGAATACGAAGAAACCAA
AGCTCTCATCTCCAT (SEQ ID NO: 54)
TATATCATTCTCCCAGAACG AGG (SEQ ID NO: 55)
TCATTCTCCCAGAACGAGGT CGG (SEQ ID NO: 56)
TTTCTTGCTAAGCATACCAG AGG (SEQ ID NO: 57)
TACCAGAGGAAACTGTTGTT TGG (SEQ ID NO: 58)
TGTTGTGCTTACTCCTTTGA AGG (SEQ ID NO: 59)
CTTCCCAGCTTCACAGTCAA TGG (SEQ ID NO: 60)
CAATGGAAACTCTCTGTCCA TGG (SEQ ID NO: 61)
CCACCAACTGTTGTGATCCA TGG (SEQ ID NO: 62)
TCCATGGAGAACCATTCGTG AGG (SEQ ID NO: 63)
GAACCATTCGTGAGGCTGAT CGG (SEQ ID NO: 64)
CGGATCAGAACCTCCATTAC CGG (SEQ ID NO: 65)
>C. sativa ACC2-2 (LOC104742086) variant1
>MEMRALVSSCSTGNGGSDPISLTNGSPWITTVGGGASTMDREFPATVKLGSSMRAFKG
VSTITVLSRTKQQFPLVCLARNNGNSTDPTSFWENDISETQR (SEQ ID NO: 66)
>5′-3′
ATGGAGATGAGAGCTTTGGTTTCTTCGTGTTCTACGGGGAATGGAGGGTCTGATCCG
ATCAGCCTCACGAATGGTTCTCCATGGATCACAACAGTTGGTGGTGGTGCAAGTACC
ATGGACAGAGAGTTTCCAGCGACTGTGAAGCTGGGAAGTAGTATGAGAGCCTTCAA
AGGAGTAAGCACAATAACAGTTCTGTCTCGGACCAAACAACAGTTTCCTCTGGTATG
CTTAGCAAGAAACAACGGAAACAGCACTGATCCGACCTCGTTCTGGGAGAACGATA
TATCTGAAACTCAAAGG (SEQ ID NO: 67)
TTTGGTTTCTTCGTGTTCTA CGG (SEQ ID NO: 68)
TTGGTTTCTTCGTGTTCTAC GGG (SEQ ID NO: 69)
TGGTTTCTTCGTGTTCTACG GGG (SEQ ID NO: 70)
TCTTCGTGTTCTACGGGGAA TGG (SEQ ID NO: 71)
TCGTGTTCTACGGGGAATGG AGG (SEQ ID NO: 72)
GATCCGATCAGCCTCACGAA TGG (SEQ ID NO: 73)
GCCTCACGAATGGTTCTCCA TGG (SEQ ID NO: 74)
TCTCCATGGATCACAACAGT TGG (SEQ ID NO: 75)
CCATGGATCACAACAGTTGG TGG (SEQ ID NO: 76)
TGGATCACAACAGTTGGTGG TGG (SEQ ID NO: 77)
TGGTGGTGGTGCAAGTACCA TGG (SEQ ID NO: 78)
GTTTCCAGCGACTGTGAAGC TGG (SEQ ID NO: 79)
AGTAGTATGAGAGCCTTCAA AGG (SEQ ID NO: 80)
GCACAATAACAGTTCTGTCT CGG (SEQ ID NO: 81)
GACCAAACAACAGTTTCCTC TGG (SEQ ID NO: 82)
GTATGCTTAGCAAGAAACAA CGG (SEQ ID NO: 83)
GCACTGATCCGACCTCGTTC TGG (SEQ ID NO: 84)
ACGATATATCTGAAACTCAA AGG (SEQ ID NO: 85)
>3′-5′
CCTTTGAGTTTCAGATATATCGTTCTCCCAGAACGAGGTCGGATCAGTGCTGTTTCCG
TTGTTTCTTGCTAAGCATACCAGAGGAAACTGTTGTTTGGTCCGAGACAGAACTGTT
ATTGTGCTTACTCCTTTGAAGGCTCTCATACTACTTCCCAGCTTCACAGTCGCTGGAA
ACTCTCTGTCCATGGTACTTGCACCACCACCAACTGTTGTGATCCATGGAGAACCAT
TCGTGAGGCTGATCGGATCAGACCCTCCATTCCCCGTAGAACACGAAGAAACCAAA
GCTCTCATCTCCAT (SEQ ID NO: 86)
TATATCGTTCTCCCAGAACG AGG (SEQ ID NO: 87)
TCGTTCTCCCAGAACGAGGT CGG (SEQ ID NO: 88)
TTTCTTGCTAAGCATACCAG AGG (SEQ ID NO: 89)
TACCAGAGGAAACTGTTGTT TGG (SEQ ID NO: 90)
TATTGTGCTTACTCCTTTGA AGG (SEQ ID NO: 91)
CTTCCCAGCTTCACAGTCGC TGG (SEQ ID NO: 92)
CGCTGGAAACTCTCTGTCCA TGG (SEQ ID NO: 93)
CCACCAACTGTTGTGATCCA TGG (SEQ ID NO: 94)
TCCATGGAGAACCATTCGTG AGG (SEQ ID NO: 95)
GAACCATTCGTGAGGCTGAT CGG (SEQ ID NO: 96)
>C. rubella ACC2(CARUB_v1GG08063mg)
>MEMRALVSSCSTGNGGSDPISLTNVSPWITTVGGGASSIDREFPTTVKLGSSLRTFKGVS
STTVLSRTKQQFPLVCLARNNANSTDPTLFWENDISEAQS (SEQ ID NO: 97)
>5′-3′
ATGGAGATGAGAGCTTTGGTTTCTTCGTGTTCTACCGGTAATGGAGGTTCTGATCCG
ATTAGCCTCACGAATGTTTCTCCATGGATCACAACAGTTGGTGGTGGTGCAAGTTCC
ATTGACAGAGAGTTTCCAACGACTGTGAAGCTGGGAAGTAGTCTGAGAACTTTCAA
AGGAGTAAGCTCTACGACAGTTTTGTCTCGGACCAAACAACAGTTTCCTCTGGTTTG
TTTAGCAAGAAACAATGCCAACAGCACTGATCCAACCTTGTTCTGGGAAAATGACAT
ATCTGAAGCTCAAAGC (SEQ ID NO: 98)
TTGGTTTCTTCGTGTTCTAC CGG (SEQ ID NO: 99)
TCTTCGTGTTCTACCGGTAA TGG (SEQ ID NO: 100)
TCGTGTTCTACCGGTAATGG AGG (SEQ ID NO: 101)
GCCTCACGAATGTTTCTCCA TGG (SEQ ID NO: 102)
TCTCCATGGATCACAACAGT TGG (SEQ ID NO: 103)
CCATGGATCACAACAGTTGG TGG (SEQ ID NO: 104)
TGGATCACAACAGTTGGTGG TGG (SEQ ID NO: 101)
GTTTCCAACGACTGTGAAGC TGG (SEQ ID NO: 105)
AGTAGTCTGAGAACTTTCAA AGG (SEQ ID NO: 106)
GCTCTACGACAGTTTTGTCT CGG (SEQ ID NO: 107)
GACCAAACAACAGTTTCCTC TGG (SEQ ID NO: 108)
GCACTGATCCAACCTTGTTC TGG (SEQ ID NO: 109)
>3′-5′
GCTTTGAGCTTCAGATATGTCATTTTCCCAGAACAAGGTTGGATCAGTGCTGTTGGC
ATTGTTTCTTGCTAAACAAACCAGAGGAAACTGTTGTTTGGTCCGAGACAAAACTGT
CGTAGAGCTTACTCCTTTGAAAGTTCTCAGACTACTTCCCAGCTTCACAGTCGTTGGA
AACTCTCTGTCAATGGAACTTGCACCACCACCAACTGTTGTGATCCATGGAGAAACA
TTCGTGAGGCTAATCGGATCAGAACCTCCATTACCGGTAGAACACGAAGAAACCAA
AGCTCTCATCTCCAT (SEQ ID NO: 110)
TATGTCATTTTCCCAGAACA AGG (SEQ ID NO: 111)
TCATTTTCCCAGAACAAGGT TGG (SEQ ID NO: 112)
CAAGGTTGGATCAGTGCTGT TGG (SEQ ID NO: 113)
TTTCTTGCTAAACAAACCAG AGG (SEQ ID NO: 114)
AACCAGAGGAAACTGTTGTT TGG (SEQ ID NO: 115)
CTTCCCAGCTTCACAGTCGT TGG (SEQ ID NO: 116)
CGTTGGAAACTCTCTGTCAA TGG (SEQ ID NO: 117)
CCACCAACTGTTGTGATCCA TGG (SEQ ID NO: 118)
TCCATGGAGAAACATTCGTG AGG (SEQ ID NO: 119)
GAAACATTCGTGAGGCTAAT CGG (SEQ ID NO: 120)
CGGATCAGAACCTCCATTAC CGG (SEQ ID NO: 121)
>B. oleracea ACC2 (LOC106301042)
>MEMRALVSCSAAGNGASDRFRLSNVSPWITSARGASGSDSPATVKLRSSSMIRAFKGV
SIYKNKTRRNVLSQRNKQFRPMAYLGRKDLSSPDPTSFCDND (SEQ ID NO: 122)
>5′-3′
ATGGAGATGAGAGCTTTGGTTTCGTGTTCTGCTGCCGGAAATGGAGCTTCTGATCGG
TTTAGACTCTCCAATGTTTCACCATGGATCACATCTGCTCGTGGTGCAAGTGGCAGT
GACTCCCCAGCCACAGTGAAGCTGAGAAGCAGCTCTATGATTAGAGCTTTCAAAGG
AGTTTCGATTTACAAAAACAAGACCAGAAGAAATGTTCTGTCTCAAAGGAACAAAC
AGTTCCGTCCTATGGCCTACTTAGGAAGGAAGGACTTGAGCAGCCCTGATCCGACCT
CCTTCTGCGATAATGAT (SEQ ID NO: 123)
TTGGTTTCGTGTTCTGCTGC CGG (SEQ ID NO: 124)
TCGTGTTCTGCTGCCGGAAA TGG (SEQ ID NO: 125)
CCGGAAATGGAGCTTCTGAT CGG (SEQ ID NO: 126)
GACTCTCCAATGTTTCACCA TGG (SEQ ID NO: 127)
CCATGGATCACATCTGCTCG TGG (SEQ ID NO: 128)
ACATCTGCTCGTGGTGCAAG TGG (SEQ ID NO: 129)
TCTATGATTAGAGCTTTCAA AGG (SEQ ID NO: 130)
GAAGAAATGTTCTGTCTCAA AGG (SEQ ID NO: 131)
GAACAAACAGTTCCGTCCTA TGG (SEQ ID NO: 132)
TTCCGTCCTATGGCCTACTT AGG (SEQ ID NO: 133)
GTCCTATGGCCTACTTAGGA AGG (SEQ ID NO: 134)
TATGGCCTACTTAGGAAGGA AGG (SEQ ID NO: 135)
3′-5′
ATCATTATCGCAGAAGGAGGTCGGATCAGGGCTGCTCAAGTCCTTCCTTCCTAAGTA
GGCCATAGGACGGAACTGTTTGTTCCTTTGAGACAGAACATTTCTTCTGGTCTTGTTT
TTGTAAATCGAAACTCCTTTGAAAGCTCTAATCATAGAGCTGCTTCTCAGCTTCACTG
TGGCTGGGGAGTCACTGCCACTTGCACCACGAGCAGATGTGATCCATGGTGAAACA
TTGGAGAGTCTAAACCGATCAGAAGCTCCATTTCCGGCAGCAGAACACGAAACCAA
AGCTCTCATCTCCAT (SEQ ID NO: 136)
TCATTATCGCAGAAGGAGGT CGG (SEQ ID NO: 137)
TCGCAGAAGGAGGTCGGATC AGG (SEQ ID NO: 138)
CAAGTCCTTCCTTCCTAAGT AGG (SEQ ID NO: 139)
TTCCTTCCTAAGTAGGCCAT AGG (SEQ ID NO: 140)
TTCCTAAGTAGGCCATAGGA CGG (SEQ ID NO: 141)
TTGAGACAGAACATTTCTTC TGG (SEQ ID NO: 143)
GCTGCTTCTCAGCTTCACTG TGG (SEQ ID NO: 144)
CTTCTCAGCTTCACTGTGGC TGG (SEQ ID NO: 145)
TTCTCAGCTTCACTGTGGCT GGG (SEQ ID NO: 146)
TCTCAGCTTCACTGTGGCTG GGG (SEQ ID NO: 147)
CCACGAGCAGATGTGATCCA TGG (SEQ ID NO: 148)
TGTGATCCATGGTGAAACAT TGG (SEQ ID NO: 149)
CCGATCAGAAGCTCCATTTC CGG (SEQ ID NO: 150)
>B. napus ACC2-1: Y10302
MEMRALVSCSAAGNGASDRFRLSNVSPWITSARGASGSDSPATVKLGSSSMIRAFKGVS
IYKNKTRRNVLSQRNKQFRPMAYLGRKDLSSPDPTSFCDNDISEPQGTGSINGNDHSAV
RVSQVDEFCKAHGGKRPIHSILVATNGMAAVKLIRSVRAWSYQTFGSEKSISLVAMATP
EDMRINAEHIRIADQFMQVPGGTNNNNYANVHLIVEMAQATGVDAVWPGWGHASENP
ELPDALKAKGVIFLGPTAASMLALGDKIGSSLIAQAADVPTLPWSGSHVKIPPGSSMVTIP
EEMYRQACVYTTEEAVASCQVVGYPAMIKASWGGGGKGIREVHDDDEVRTLFKQVQG
EVPGSPIFIMKVASQSRHL (SEQ ID NO: 151)
>5′-3′ 1^{ST} EXON
ATGGAGATGAGAGCTTTAGTTTCGTGTTCTGCTGCCGGAAATGGAGCTTCTGATCGG
TTTAGACTCTCCAATGTTTCACCATGGATCACATCAGCTCGTGGTGCAAGTGGCAGT
GACTCCCCAGCCACAGTGAAGCTGGGAAGCAGCTCTATGATTAGAGCTTTCAAAGG
CGTTTCGATTTACAAAAACAAGACCAGAAGGAATGTTCTGTCTCAAAGGAACAAAC
AGTTCCGTCCTATGGCCTACTTAGGAAGGAAGGACTTGAGCAGCCCTGATCCGACCT
CCTTCTGCGATAATG (SEQ ID NO: 152)
TTAGTTTCGTGTTCTGCTGC CGG (SEQ ID NO: 153)
TCGTGTTCTGCTGCCGGAAA TGG (SEQ ID NO: 154)
CCGGAAATGGAGCTTCTGAT CGG (SEQ ID NO: 155)
GACTCTCCAATGTTTCACCA TGG (SEQ ID NO: 156)
CCATGGATCACATCAGCTCG TGG (SEQ ID NO: 157)
ACATCAGCTCGTGGTGCAAG TGG (SEQ ID NO: 158)
CTCCCCAGCCACAGTGAAGC TGG (SEQ ID NO: 159)
TCCCCAGCCACAGTGAAGCT GGG (SEQ ID NO: 160)
TCTATGATTAGAGCTTTCAA AGG (SEQ ID NO: 161)
TTTACAAAAACAAGACCAGA AGG (SEQ ID NO: 162)
GAAGGAATGTTCTGTCTCAA AGG (SEQ ID NO: 163)
GAACAAACAGTTCCGTCCTA TGG (SEQ ID NO: 164)
TTCCGTCCTATGGCCTACTT AGG (SEQ ID NO: 165)
GTCCTATGGCCTACTTAGGA AGG (SEQ ID NO: 166)
TATGGCCTACTTAGGAAGGA AGG (SEQ ID NO: 167)
>3′-5′
CATTATCGCAGAAGGAGGTCGGATCAGGGCTGCTCAAGTCCTTCCTTCCTAAGTAGG
CCATAGGACGGAACTGTTTGTTCCTTTGAGACAGAACATTCCTTCTGGTCTTGTTTTT
GTAAATCGAAACGCCTTTGAAAGCTCTAATCATAGAGCTGCTTCCCAGCTTCACTGT
GGCTGGGGAGTCACTGCCACTTGCACCACGAGCTGATGTGATCCATGGTGAAACATT
GGAGAGTCTAAACCGATCAGAAGCTCCATTTCCGGCAGCAGAACACGAAACTAAAG
CTCTCATCTCCAT (SEQ ID NO: 168)
TCGCAGAAGGAGGTCGGATC AGG (SEQ ID NO: 169)
CGCAGAAGGAGGTCGGATCA GGG (SEQ ID NO: 170)
CAAGTCCTTCCTTCCTAAGT AGG (SEQ ID NO: 171)
TTCCTTCCTAAGTAGGCCAT AGG (SEQ ID NO: 172)
TTCCTAAGTAGGCCATAGGA CGG (SEQ ID NO: 173)
TTGAGACAGAACATTCCTTC TGG (SEQ ID NO: 174)
CTTCCCAGCTTCACTGTGGC TGG (SEQ ID NO: 175)
TTCCCAGCTTCACTGTGGCTGGG (SEQ ID NO: 176)
TCCCAGCTTCACTGTGGCTG GGG (SEQ ID NO: 177)
CCACGAGCTGATGTGATCCA TGG (SEQ ID NO: 178)
TGTGATCCATGGTGAAACAT TGG (SEQ ID NO: 179)
CCGATCAGAAGCTCCATTTC CGG (SEQ ID NO: 180)
>B. napus ACC2-2: X77576
MEMRALVSCSAAGNGASDRFRLSNVSPWITSARGASGSDSPATVKLGSSSMIRAFKGVS
IYKNKTRRNVLSQRNKQFRPMAYLGRKDLSSPDPTSFCDNDISEPQGTGSINGNDHSAV
RVSQVDEFCKAHGGKRPIHRILVATNGMAAVKFIRSVRAWSYQTFGSEKSISLVAMATP
EDMRINAEHIRIADQFMQVPGGTNNNNYANVHLIVEMAEATGVDAVWPGWGHASENP
ELPDALKAKGVIFLGPTAASMLALGDKIGSSLIAQAADVPTLPWSGSHVKIPPGSSLVTIP
EEMYRQACVYTTEEAVASCQVVGYPAMIKASWGGGGKGIRKVHDDDEVRALFKQVQ
GEVPGSPIFIMKVASQSRHLEVQLLCDQYGNVSALHSRDCSVQRRHQKIIEEGPITVAPR
DTVKKLEQAARRLAKSVNYVGAATVEFLYSMDTGDYFFLELNPR (SEQ ID NO: 181)
>5′-3′ 1^{ST} EXON
ATGGAGATGAGAGCTTTGGTTTCGTGTTCTGCTGCCGGAAATGGAGCTTCTGATCGG
TTTAGACTCTCCAATGTTTCACCATGGATCACATCAGCTCGTGGTGCAAGTGGCAGT
GACTCCCCAGCCACAGTGAAGCTGGGAAGCAGCTCTATGATCAGAGCCTTCAAAGG
AGTTTCGATTTACAAAAACAAGACCAGAAGAAATGTTTTGTCTCAAAGGAACAAAC
AGTTTCGTCCTATGGCCTACTTAGGAAGGAAGGACTTGAGCAGCCCTGATCCGACCT
CCTTCTGCGATAATG (SEQ ID NO: 182)
TTGGTTTCGTGTTCTGCTGC CGG (SEQ ID NO: 183)
TCGTGTTCTGCTGCCGGAAA TGG (SEQ ID NO: 184)
CCGGAAATGGAGCTTCTGAT CGG (SEQ ID NO: 185)
GACTCTCCAATGTTTCACCA TGG (SEQ ID NO: 186)
CCATGGATCACATCAGCTCG TGG (SEQ ID NO: 187)
ACATCAGCTCGTGGTGCAAG TGG (SEQ ID NO: 188)
CTCCCCAGCCACAGTGAAGC TGG (SEQ ID NO: 189)
TCCCCAGCCACAGTGAAGCT GGG (SEQ ID NO: 190)
TCTATGATCAGAGCCTTCAA AGG (SEQ ID NO: 191)
GAAGAAATGTTTTGTCTCAA AGG (SEQ ID NO: 192)
GAACAAACAGTTTCGTCCTA TGG (SEQ ID NO: 193)
TTTCGTCCTATGGCCTACTT AGG (SEQ ID NO: 194)
GTCCTATGGCCTACTTAGGA AGG (SEQ ID NO: 195)
TATGGCCTACTTAGGAAGGA AGG (SEQ ID NO: 196)
>3′-5′
CATTATCGCAGAAGGAGGTCGGATCAGGGCTGCTCAAGTCCTTCCTTCCTAAGTAGG
CCATAGGACGAAACTGTTTGTTCCTTTGAGACAAAACATTTCTTCTGGTCTTGTTTTT
GTAAATCGAAACTCCTTTGAAGGCTCTGATCATAGAGCTGCTTCCCAGCTTCACTGT
GGCTGGGGAGTCACTGCCACTTGCACCACGAGCTGATGTGATCCATGGTGAAACATT
GGAGAGTCTAAACCGATCAGAAGCTCCATTTCCGGCAGCAGAACACGAAACCAAAG
CTCTCATCTCCAT (SEQ ID NO: 197)
TCGCAGAAGGAGGTCGGATC AGG (SEQ ID NO: 198)
CGCAGAAGGAGGTCGGATCA GGG (SEQ ID NO: 199)
CAAGTCCTTCCTTCCTAAGT AGG (SEQ ID NO: 200)
TTCCTTCCTAAGTAGGCCAT AGG (SEQ ID NO: 201)
TTGAGACAAAACATTTCTTC TGG (SEQ ID NO: 202)
GTAAATCGAAACTCCTTTGA AGG (SEQ ID NO: 203)
GCTGCTTCCCAGCTTCACTG TGG (SEQ ID NO: 204)
CTTCCCAGCTTCACTGTGGC TGG (SEQ ID NO: 205)
TTCCCAGCTTCACTGTGGCT GGG (SEQ ID NO: 206)
TCCCAGCTTCACTGTGGCTG GGG (SEQ ID NO: 207)
CCACGAGCTGATGTGATCCA TGG (SEQ ID NO: 208)
TGTGATCCATGGTGAAACAT TGG (SEQ ID NO: 209)
CCGATCAGAAGCTCCATTTC CGG (SEQ ID NO: 210)
>B. rapa ACC2 (LOC1038715GG)
>MEMRALVSCSAAGNGASDRFRLSNVSPWITSARGASGSDSPATVKLGSSSMIRAFKGV
SIYKNKSRRNVLSQRNKQFRPMAYLGRKDLSSPDPTSFCDND (SEQ ID NO: 211)
>5′-3′
ATGGAGATGAGAGCTTTGGTTTCGTGTTCTGCTGCCGGAAATGGAGCTTCTGATCGG
TTTAGACTCTCCAATGTTTCACCATGGATCACATCAGCTCGTGGTGCAAGTGGCAGT
GACTCCCCAGCCACAGTGAAGCTGGGAAGCAGCTCTATGATCAGAGCCTTCAAAGG
AGTTTCGATTTACAAAAACAAGAGCAGAAGAAATGTTCTGTCTCAAAGGAACAAAC
AGTTTCGTCCTATGGCCTACTTAGGAAGGAAGGACTTGAGCAGCCCTGATCCGACCT
CCTTCTGCGATAATGAT (SEQ ID NO: 212)
TTGGTTTCGTGTTCTGCTGC CGG (SEQ ID NO: 213)
TCGTGTTCTGCTGCCGGAAA TGG (SED ID NO: 214)
CCGGAAATGGAGCTTCTGAT CGG (SED ID NO: 215)
GACTCTCCAATGTTTCACCA TGG (SED ID NO: 216)
CCATGGATCACATCAGCTCG TGG (SED ID NO: 217)
ACATCAGCTCGTGGTGCAAG TGG (SED ID NO: 218)
CTCCCCAGCCACAGTGAAGC TGG (SED ID NO: 219)
TCTATGATCAGAGCCTTCAA AGG (SED ID NO: 220)
GAAGAAATGTTCTGTCTCAA AGG (SED ID NO: 221)
TTTCGTCCTATGGCCTACTT AGG (SED ID NO: 223)
GTCCTATGGCCTACTTAGGA AGG (SED ID NO: 224)
TATGGCCTACTTAGGAAGGA AGG (SED ID NO: 225)
3′-5′
ATCATTATCGCAGAAGGAGGTCGGATCAGGGCTGCTCAAGTCCTTCCTTCCTAAGTA
GGCCATAGGACGAAACTGTTTGTTCCTTTGAGACAGAACATTTCTTCTGCTCTTGTTT
TTGTAAATCGAAACTCCTTTGAAGGCTCTGATCATAGAGCTGCTTCCCAGCTTCACT
GTGGCTGGGGAGTCACTGCCACTTGCACCACGAGCTGATGTGATCCATGGTGAAAC
ATTGGAGAGTCTAAACCGATCAGAAGCTCCATTTCCGGCAGCAGAACACGAAACCA
AAGCTCTCATCTCCAT (SED ID NO: 226)
TCATTATCGCAGAAGGAGGT CGG (SED ID NO: 227)
TCGCAGAAGGAGGTCGGATC AGG (SED ID NO: 228)
CAAGTCCTTCCTTCCTAAGT AGG (SED ID NO: 229)
TTCCTTCCTAAGTAGGCCAT AGG (SED ID NO: 230)
GTAAATCGAAACTCCTTTGA AGG (SED ID NO: 231)
GCTGCTTCCCAGCTTCACTG TGG (SED ID NO: 232)
CTTCCCAGCTTCACTGTGGC TGG (SED ID NO: 233)
TTCCCAGCTTCACTGTGGCT GGG (SED ID NO: 234)
TCCCAGCTTCACTGTGGCTG GGG (SED ID NO: 235)
CCACGAGCTGATGTGATCCA TGG (SED ID NO: 236)
TGTGATCCATGGTGAAACAT TGG (SED ID NO: 237)
CCGATCAGAAGCTCCATTTC CGG (SED ID NO: 238)

Deletion of ACC2 Gene in Regenerable RLD and Ws Arabidopsis ecotypes

The bacterial clustered regularly interspaced short palindromic repeats (CRISPR) and CRISPR-associated (Cas) defense system has been rapidly developed as a genome-engineering tool (Belhaj et al., 2013; Mali et al., 2013; Li et al., 2014). In this approach a small RNA guides the Cas9 nuclease to the target site. The nick is then repaired by non-homologous end joining, the process most often resulting in a one-nucleotide insertion or deletion in Arabidopsis thaliana (Feng et al., 2014). Because our objective is knocking out the ACC2 gene, we used the same system, an Agrobacterium binary transformation vector in which the sgRNA is transcribed under the control of Arabidopsis U6 snoRNA promoter (pAtU6) and Cas9 is expressed from the Arabidopsis ubiquitin promoter (pAtUBQ1) (Mao et al., 2013).

The 16 guide strands provided below are suitable for this approach.

sequence
(SEQ ID NO: 239)
TCCATGCAGATATATTCGTG AGG
(SEQ ID NO: 240)
CCCTCACGAATATATCTCCA TGG
( [symbol] EQ ID NO: 241)
GATATATTCGTGAGGGTAAT TGG
(SEQ ID NO: 242)
CTTCTCAGCTTCACTGTCGA CGG
(SEQ ID NO: 243)
CCATGGAGATATATTCGTGA GGG
(SEQ ID NO: 244)
CTTCGACAAGAGTTTTGTCT CGG
(SEQ ID NO: 245)
TCAAGAGTGAGAACCTTTAA AGG
(SEQ ID NO: 246)
TCTTCGTGTTCTACTGGTAA TGG
(SEQ ID NO: 247)
TTGGGTTCTTCGTGTTCTAC TGG
(SEQ ID NO: 248)
TGTCGAAGAAACTCCTTTAA AGG
(SEQ ID NO: 249)
TCTTGACGAAACTCCTTTGA AGG
(SEQ ID NO: 250)
AGTAGTTTGAGAACCTTCAA AGG
(SEQ ID NO: 251)
TCGTGTTCTACTGGTAATGG AGG
(SEQ ID NO: 252)
GGAAAAACTGTTGTGATCCA TGG
(SEQ ID NO: 253)
AAACAGAGGAAACTGTTGTT TGG
(SEQ ID NO: 254)
GGGTTTAGGAAACAAAACAG AGG

The target for mutagenesis wase exon-1 of the ACC2 coding region encoding the chloroplast transit peptide. This N-terminal extension is absent in the ACC1 gene, which targets its product to the cytoplasm. To design the targeting region of the guide RNA, 240 nucleotides of ACC2 exon-1 were pasted into the guide RNA design at the MIT Optimized CRISPR Design website. The RLD and Ws sequence has a one-nucleotide (A instead of G) mismatch compared to Columbia, a sequence variation that was be considered when designing the sgRNA. The closest off target site in the Arabidopsis genome has three mismatches with this target site.

To target the ACC2 sequence CCCTCACGAATATATCTCCATGG (2^{nd} target site in the 10 list; SEQ ID NO: 240), we cloned two annealed oligonucleotides that form the target site in BbsI-digested CRISPR/Cas9 cassette psgR-Cas9-At (Mao et al., 2013). The oligonucleotides were gattgCCTCACGAATATATCTCCA (SEQ ID NO: 255), and aaacTGGAGATATATTCGTGAGGc (SEQ ID NO: 256). The CRISPR/Cas9 cassette was then cloned in a pCAMBIA2300 Agrobacterium binary vector and introduced into Arabidopsis by the flower dip protocol (Clough and Bent, 1998). Plants transformed with the CRISPR/Cas9 construct were selected by germinating seeds on kanamycin medium (100 mg/L).

Kanamycin resistant seedlings (T1 generation) were screened for a mutant ACC2 target site by the T7 exonuclease I (T7E1) assay (Xie and Yang, 2013). The T7 endonuclease recognizes and cleaves non-perfectly matched DNA. The ACC2 target region was PCR amplified using forward primer 5′-TCTCTTCCTCCTTAAAAAGCCACA-3′ (SEQ ID NO: 257) and reverse primer 5′-CTAGGATTCGAAACCAGCGT-3′ (SEQ ID NO: 258) using total cellular DNA as template, the amplicons were denatured, reannealed and treated with T7E1. Mismatch caused by CRISPR/Cas9 mutagenesis resulted in T7E1 cleaving the mismatched DNA, that was visualized by gel electrophoresis.

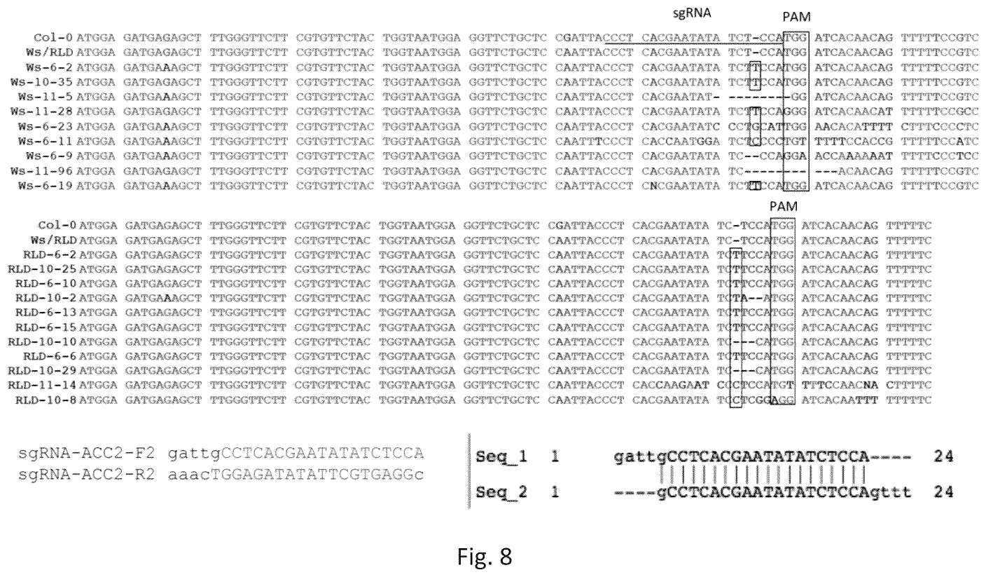

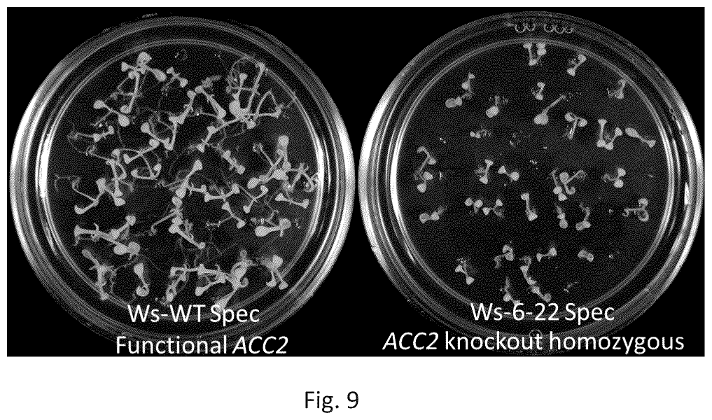

Plants carrying mutations in ACC2 gene copies were identified by T7E1 screening the heterozygous T1 seed progeny. Mutations in ACC2 genes were identified in the T2 generation by sequencing PCR amplicons (FIG. 8). The acc2 knockout mutants, in contrast to wild type, do not develop shoot meristem outgrowths when germinated on spectinomycin (Parker et al., 2014). Therefore, we collected seed from the T1 plants, and germinated a small sample on spectinomycin medium to identify non-segregating acc2 knockout populations by spectinomycin sensitivity. An example for seedling spectinomycin hypersensitive reaction is shown in FIG. 9. Note development of primary leaves on the seedlings of the parental Ws line, and the absence of any shoot meristem outgrowth on the hypersensitive Ws-2-22 mutant (Parker et al., 2014). Following this protocol uniform, non-segregating RLD and Ws seed was obtained. Such spectinomycin hypersensitive plants are the suitable recipients for plastid transformation.

### Example III

### Expression of Heterologous Genes in ACC2-Defective Brassica spp

Reproducible, high-frequency plastid transformation in the Brassicae oilseed and vegetable crops enables plastid genome engineering in spectinomycin hypersensitive Brassica spp. for a variety of biotechnological applications.

One application is replacement of part or the entire plastid genome with synthetic DNA. For example, the efficiency of sunlight to biomass conversion can be improved by introducing genes or groups of genes from other crop species, algae, and photosynthetic bacteria (Gimpel et al., 2016; Hanson et al., 2016; Sharwood et al., 2016).

Expression of plastid transgenes throughout the plant is desirable for some applications, for example tolerance to herbicides such as phosphinothricin (PPT) (Lutz et al., 2001; Ye et al., 2003), glyphosate (Ye et al., 2003), sulfonylurea, pyrimidinylcarboxylate (Shimizu et al., 2008) and diketonitrile (Dufourmantel et al., 2007). Equally useful are plastid expression of insecticidal protein genes (U.S. Pat. No. 5,545,818) and double-stranded RNAs that are toxic to insects (Zhang et al., 2015). The herbicide resistance and insecticidal genes are introduced by linkage to the selective spectinomycin resistance (aadA gene) marker. When uniform transformation of plastid genomes is obtained, the marker gene can be excised by a site-specific recombinase that targets sites flanking the marker gene. Various marker excision systems are suitable including the Cre/IoxP or PhiC31/Int systems (as described in U.S. Pat. Nos. 7,217,860 and 8,841,511) or the BxB1 (Shao et al., 2014), ParA-MRS, and CinH-Rs2 (Shao et al., 2017) site-specific recombination systems.

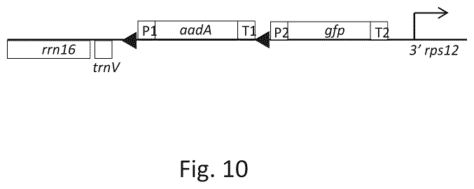

Particularly effective for the recovery of transplastomic events are the PrrnLatpB/TrbcL, PrrnLatpB/TpsbA, PrrnLrbcL/TpsbA, PrrnLT7g10/TrbcL promoter/terminator cassettes (Kuroda and Maliga, 2001, 2001). Genes of interest may also be expressed using cassettes previously described in U.S. Pat. Nos. 5,977,402, 6,297,054, 6, 376, 744, 6,472,568, 6,624,296, 6,987,215, 7,176,355, 8,143,474. FIG. 10 shows a schematic design of a plastid transformation vector having a Brassica napus plastid targeting sequence containing the rrn16 targeting region (nucleotides 135473-137978 in GenBank accession KP161617) and carrying a recombinase target site-flanked selectable aadA marker and a gene of interest.

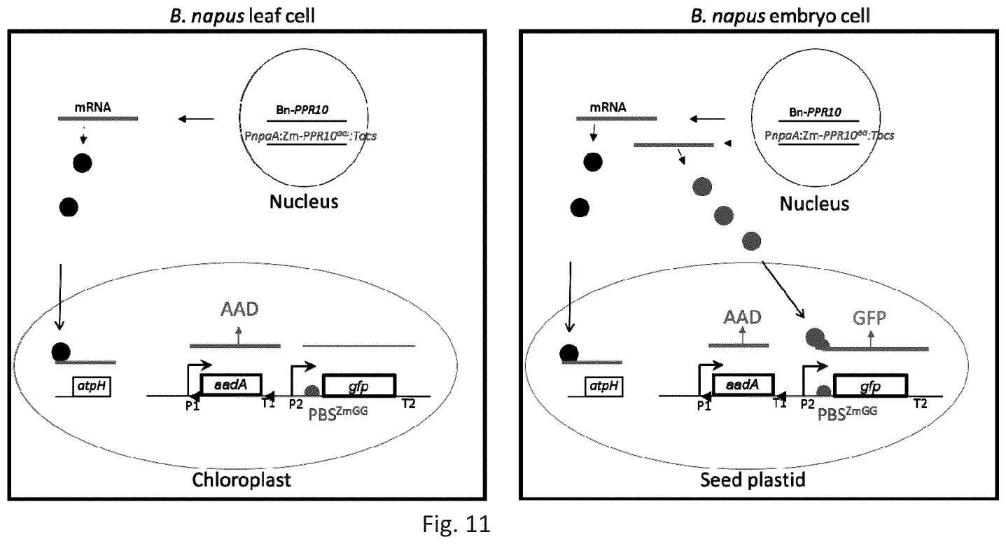

Tissue-specific expression of plastid genes is desirable but thus far no practical system has been available to achieve this objective. We describe here seed-specific expression of proteins in plastids based on a transgene incorporated in the plastid genome that is regulated by a nuclear gene with a seed-specific promoter. The elements of the system are depicted in FIG. 11A. In a Brassica spp. the transgene encoding green fluorescent protein (or particular gene of interest) is present in the leaf cell, but is not translated in the absence of a modified PPR10 RNA binding protein. The engineered Zea mays PPR 10GG protein gene that is required for expression is present in the nucleus, but is not active because it is under the control of a seed-specific Brassica napus napin gene promoter that is not transcribed in the nucleus of leaf cells (Ellerstrom et al., 1996). The native Brassica PPR10 protein (Bn-PPR10) stabilizes and facilitates translation of the atpH mRNA. However, Bn-PPR10 RNA binding protein does not recognize PBS^{ZmGG}, the mutant maize PPR10 binding site because the 23-nucleotide Brassica binding site differs by 2 nucleotides from the wild-type maize PPR10 binding site and by 4 nucleotides from the mutant maize binding site.

Zm-PPR10 wt Binding site:
(SEQ ID NO: 259)
ATTGTATCcTTAACcATTTCTTT
Bn-PPR10 wt Binding site:
(SEQ ID NO: 260)
ATTGTATCATTAACTATTTCTTT
Zm-PPR10^{GG} mut Binding site:
(SEQ ID NO: 261)
ATTGTAggcTTAACcATTTCTTT

In the Brassica ssp. seed (embryo) cell, the napin seed storage protein gene promoter is turned on, the mRNA is translated in the cytoplasm and the PPR10GG protein is imported into chloroplasts where it binds to its cognate binding site upstream of the gfp AUG translation initiation codon. Binding of the Zm-PPR 10^{GG} stabilizes the gfp mRNA and facilitates its translation. The result is high-level GFP protein accumulation in the plastids of embryo cells in oilseed crops.

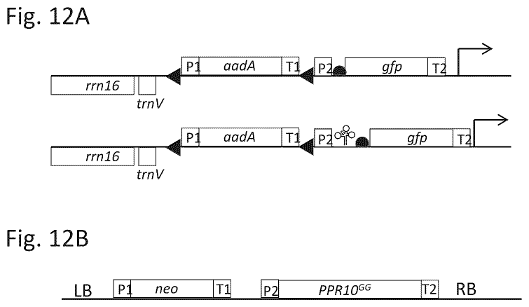

To construct the regulated plastid transgenes, the tobacco Prrn promoter is linked up with the 100 nt sequence directly upstream of the maize atpH gene. The two sequences together constitute the 5′ regulatory region driving GFP expression. The gfp coding region is followed by the rbc gene terminator (TrbcL). Prrn-PPR10^{GG}-GFP-TrbcL corresponds to SEQ ID NO. 262. The transgene is cloned adjacent to an aadA gene in the B. napus-specific plastid transformation vector shown in FIG. 12A. Also shown in FIG. 12A is a variant, where a T-RNA (symbolized with a cloverleaf; SEQ ID NO. 263) is cloned between the promoter and the 100 nt maize sequence. The tRNA is efficiently processed to create a processed end that is more sensitive to degradation in the absence of the protecting Zm-PPR10^{GG} protein, reducing background in the absence of the PPR10^{GG} protein. This construct can be engineered to express a protein of interest in the place of GFP, or a protein of interest can be operably linked to GFP via cleavable protein linker.

Likewise, a Brassica napus seed-specific PnpaA:PPR10^{GG}:Tocs nuclear transgene can be cloned into a pCAMBIA2300 Agrobacterium binary vector with a plant-selectable kanamycin resistance gene for transformation of the B. napus nucleus (FIG. 12B). The modified Zea mays PPR10 gene sequence that results in selective recognition of the modified GG RNA binding site is described (Barkan et al., 2012) (SEQ ID NO: 265). For reference, the wild-type maize PPR10 sequence is also listed (SEQ ID NO: 264). The PPR10 protein is naturally targeted to chloroplasts, thus it requires only a tissue-specific promoter and a eukaryotic transcription terminator, such as octopine synthase 3′ UTR (Tocs) (GenBank accession no. AJ311872.1). The napin gene is encoded in a small gene family. A suitable promoter for the PnpaA:PPR10^{GG}. Tocs gene was characterized experimentally (Ellerstrom et al., 1996) (GenBank accession J02798), and additional B. napus promoters are available (Sohrabi et al., 2015). The promoter of a legume storage protein gene, phaseolin, (SEQ ID NO: 266) is known to be very efficient for the expression of recombinant proteins in Arabidopsis thaliana (De Jaegert et al., 2002). The promoter sequence is available in US patent application 2003/0159183.

>Prrn-PPR10GG-GFP-TrbcL
SEQ ID NO: 262
>GagctcGCTCCCCCGCCGTCGTTCAATGAGAATGGATAAGAGGCTCGTGGGATTGAC
GTGAGGGGGCAGGGATGGCTATATTTCTGGGAGTTACTTCTACCCGATAGAGCTTAG
AAGTTGGAAGTAATAATTTCTTGGTTGATTGTAGGCTTAACCATTTCTTTTTTTTTGA
CACGAGGAACTCATCATGgctagcAGTAAAGGAGAAGAACTTTTCACTGGAGTTGTCCC
AATTCTTGTTGAATTAGATGGTGATGTTAATGGGCACAAATTTTCTGTCAGTGGAGA
GGGTGAAGGTGATGCAACATACGGAAAACTTACCCTTAAATTTATTTGCACTACTGG
AAAACTACCTGTTCCtTGGCCAACACTTGTCACTACTTTCTCTTATGGTGTTCAATGCT
TTTCAAGATACCCAGATCATATGAAGCGGCACGACTTCTTCAAGAGCGCCATGCCTG
AGGGATACGTGCAGGAGAGGACCATCTCTTTCAAGGACGACGGGAACTACAAGACA
CGTGCTGAAGTCAAGTTTGAGGGAGACACCCTCGTCAACAGGATCGAGCTTAAGGG
AATCGATTTCAAGGAGGACGGAAACATCCTCGGCCACAAGTTGGAATACAACTACA
ACTCCCACAACGTATACATCACGGCAGACAAACAAAAGAATGGAATCAAAGCTAAC
TTCAAAATTAGACACAACATTGAAGATGGAAGCGTTCAACTAGCAGACCATTATCA
ACAAAATACTCCAATTGGCGATGGCCCTGTCCTTTTACCAGACAACCATTACCTGTC
CACACAATCTGCCCTTTCGAAAGATCCCAACGAAAAGAGAGACCACATGGTCCTTCT
TGAGTTTGTAACAGCTGCTGGGATTACACATGGCATGGATGAACTgTACAAATAAAtc
tagaAAACAGTAGACATTAGCAGATAAATTAGCAGGAAATAAAGAAGGATAAGGAGA
AAGAACTCAAGTAATTATCCTTCGTTCTCTTAATTGAATTGCAATTAAACTCGGCCC
AATCTTTTACTAAAAGGATTGAGCCGAATACAACAAAGATTCTATTGCATATATTTT
GACTAAGTATATACTTACCTAGATATACAAGATTTGAAATACAAAATCTAaagctt
>tRNA (trnP, 147 bp)
SEQ ID NO: 263
AAGTCTTTACAATGACAATGGAAACCGATGTAAAGGGATGTAGCGCAGCTTGGTAG
CGCGTTTGTTTTGGGTACAAAATGTCACAGGTTCAAATCCTGTCATCCCTATCCCTAA
CTtGTAGTTATCGTATCAGCAGTAACAATAGAT (SEQ ID NO: 263)
>Zm_PPR10 WT
SEQ ID NO: 264
ATGGAGGCCACCGGCAGGGGGCTGTTCCCGAACAAGCCCACCCTCCCGGCGGGGCC
GAGGAAACGGGGCCCGCTCCTCCCGGCCGCGCCCCCGCCACCGTCCCCCTCCTCGCT
CCCGCTCGACTCGCTCCTGCTCCACCTCACCGCGCCCGCCCCCGCGCCGGCCCCCGC
GCCGCGGCGGTCGCACCAGACGCCGACGCCGCCGCACTCCTTCCTCTCCCCCGACGC
GCAGGTGCTGGTGCTCGCCATCTCCTCGCACCCGCTCCCCACGCTGGCGGCCTTCCT
GGCCTCCCGCCGCGACGAGCTCCTCCGCGCGGACATCACGTCCCTGCTCAAGGCGCT
GGAGCTCTCGGGGCACTGGGAGTGGGCGCTCGCGCTCCTCCGGTGGGCAGGCAAGG
AGGGTGCCGCCGACGCGTCGGCGCTCGAGATGGTCGTCCGCGCGCTGGGCCGCGAG
GGCCAGCACGACGCCGTCTGCGCGCTGCTCGACGAAACGCCGCTCCCGCCGGGCTC
CCGCCTCGACGTCCGCGCCTACACCACCGTGCTGCACGCGCTCTCCCGCGCGGGCCG
GTACGAGCGCGCGCTCGAGCTCTTCGCCGAGCTCCGGCGCCAGGGGGTGGCGCCCA
CGCTCGTCACCTACAACGTCGTGCTGGACGTGTACGGGCGGATGGGCCGGTCGTGG
CCGCGGATCGTCGCCCTCCTCGATGAGATGCGCGCCGCCGGGGTCGAGCCCGACGG
CTTCACCGCCAGCACGGTGATCGCCGCGTGCTGCCGCGACGGGCTGGTTGACGAGG
CGGTGGCGTTCTTCGAGGACCTCAAGGCCCGCGGCCACGCCCCGTGCGTCGTCACGT
ACaacGCGTTGCTCCAGGTGTTCGGCAAGGCCGGGAACTACACGGAGGCGCTGCGCG
TGCTCGGGGAGATGGAGCAGAACGGCTGCCAGCCAGATGCTGTGACGTACaacGAGC
TCGCCGGAACGTACGCCCGGGCTGGGTTCTTCGAGGAGGCTGCCAGGTGCCTGGAC
ACAATGGCATCCAAGGGTCTGTTGCCAAACGCATTCACGTACAACACCGTGATGAC
AGCCTATGGGAATGTTGGGAAGGTGGATGAGGCGCTCGCTCTGTTTGACCAGATGA
AGAAGACCGGGTTCGTGCCGAACGTGAACACGTACAATCTTGTCCTTGGCATGCTTG
GCAAGAAGTCAAGGTTCACGGTGATGCTAGAGATGCTTGGAGAGATGTCGAGGAGC
GGATGCACACCGAACCGGGTAACATGGAACACAATGCTTGCAGTCTGTGGGAAGCG
TGGCATGGAGGACTACGTCACCCGGGTTCTGGAGGGGATGAGGTCTTGCGGGGTTG
AACTGAGCCGAGACACCTACAACACCCTGATAGCTGCGTACGGCCGGTGTGGCTCG
AGGACTAATGCCTTCAAGATGTACAACGAGATGACCAGCGCTGGATTCACCCCCTG
CATCACCACGTACAACGCGTTGCTGAACGTGCTGTCGCGGCAGGGCGACTGGTCCA
CCGCCCAGTCGATCGTAAGCAAAATGAGGACCAAGGGGTTCAAGCCGAACGAGCAG
TCGTATTCGCTGCTGCTCCAGTGCTACGCGAAGGGGGGCAACGTGGCAGGGATAGC
CGCGATCGAGAACGAGGTGTACGGATCAGGTGCCGTTTTCCCAAGCTGGGTGATCCT
GAGGACCCTTGTCATCGCCAATTTCAAGTGCCGGCGACTGGATGGCATGGAGACGG
CGTTTCAAGAGGTGAAGGCCAGAGGCTACAACCCGGACCTCGTGATATTCAACTCC
ATGCTGTCCATCTACGCGAAGAACGGGATGTACAGCAAGGCCACCGAGGTCTTCGA
CTCCATCAAGCGGAGCGGGCTGAGCCCCGACCTCATCACCTACAACAGCCTGATGG
ACATGTACGCCAAGTGCAGCGAGTCGTGGGAGGCCGAGAAGATACTGAACCAGCTC
AAGTGCTCCCAGACGATGAAGCCCGACGTGGTGTCCTACAACACGGTCATAAACGG
GTTCTGCAAGCAGGGGCTGGTGAAAGAGGCCCAGAGGGTCCTCTCGGAGATGGTCG
CCGACGGCATGGCCCCCTGCGCCGTGACCTACCACACGCTCGTCGGGGGTTACTCCA
GCCTGGAGATGTTCAGCGAGGCCAGGGAGGTCATCGGCTACATGGTCCAGCACGGC
CTCAAGCCTATGGAGCTGACCTACAGGAGAGTCGTCGAGAGCTACTGCAGAGCGAA
GCGGTTCGAGGAGGCTCGCGGCTTCCTGTCCGAGGTCTCGGAGACCGACCTGGATTT
TGACAAGAAGGCGCTCGAAGCCTATATAGAGGATGCGCAGTTTGGAAGGTAG (SEQ
>Zm_PPR10 GG
SEQ ID NO: 265
ATGGAGGCCACCGGCAGGGGGCTGTTCCCGAACAAGCCCACCCTCCCGGCGGGGCC
GAGGAAACGGGGCCCGCTCCTCCCGGCCGCGCCCCCGCCACCGTCCCCCTCCTCGCT
CCCGCTCGACTCGCTCCTGCTCCACCTCACCGCGCCCGCCCCCGCGCCGGCCCCCGC
GCCGCGGCGGTCGCACCAGACGCCGACGCCGCCGCACTCCTTCCTCTCCCCCGACGC
GCAGGTGCTGGTGCTCGCCATCTCCTCGCACCCGCTCCCCACGCTGGCGGCCTTCCT
GGCCTCCCGCCGCGACGAGCTCCTCCGCGCGGACATCACGTCCCTGCTCAAGGCGCT
GGAGCTCTCGGGGCACTGGGAGTGGGCGCTCGCGCTCCTCCGGTGGGCAGGCAAGG
AGGGTGCCGCCGACGCGTCGGCGCTCGAGATGGTCGTCCGCGCGCTGGGCCGCGAG
GGCCAGCACGACGCCGTCTGCGCGCTGCTCGACGAAACGCCGCTCCCGCCGGGCTC
CCGCCTCGACGTCCGCGCCTACACCACCGTGCTGCACGCGCTCTCCCGCGCGGGCCG
GTACGAGCGCGCGCTCGAGCTCTTCGCCGAGCTCCGGCGCCAGGGGGTGGCGCCCA
CGCTCGTCACCTACAACGTCGTGCTGGACGTGTACGGGCGGATGGGCCGGTCGTGG
CCGCGGATCGTCGCCCTCCTCGATGAGATGCGCGCCGCCGGGGTCGAGCCCGACGG
CTTCACCGCCAGCACGGTGATCGCCGCGTGCTGCCGCGACGGGCTGGTTGACGAGG
CGGTGGCGTTCTTCGAGGACCTCAAGGCCCGCGGCCACGCCCCGTGCGTCGTCACGT
ACacaGCGTTGCTCCAGGTGTTCGGCAAGGCCGGGAACTACACGGAGGCGCTGCGCG
TGCTCGGGGAGATGGAGCAGAACGGCTGCCAGCCAGATGCTGTGACGTACaccGAGC
TCGCCGGAACGTACGCCCGGGCTGGGTTCTTCGAGGAGGCTGCCAGGTGCCTGGAC
ACAATGGCATCCAAGGGTCTGTTGCCAAACGCATTCACGTACAACACCGTGATGAC
AGCCTATGGGAATGTTGGGAAGGTGGATGAGGCGCTCGCTCTGTTTGACCAGATGA
AGAAGACCGGGTTCGTGCCGAACGTGAACACGTACAATCTTGTCCTTGGCATGCTTG
GCAAGAAGTCAAGGTTCACGGTGATGCTAGAGATGCTTGGAGAGATGTCGAGGAGC
GGATGCACACCGAACCGGGTAACATGGAACACAATGCTTGCAGTCTGTGGGAAGCG
TGGCATGGAGGACTACGTCACCCGGGTTCTGGAGGGGATGAGGTCTTGCGGGGTTG
AACTGAGCCGAGACACCTACAACACCCTGATAGCTGCGTACGGCCGGTGTGGCTCG
AGGACTAATGCCTTCAAGATGTACAACGAGATGACCAGCGCTGGATTCACCCCCTG
CATCACCACGTACAACGCGTTGCTGAACGTGCTGTCGCGGCAGGGCGACTGGTCCA
CCGCCCAGTCGATCGTAAGCAAAATGAGGACCAAGGGGTTCAAGCCGAACGAGCAG
TCGTATTCGCTGCTGCTCCAGTGCTACGCGAAGGGGGGCAACGTGGCAGGGATAGC
CGCGATCGAGAACGAGGTGTACGGATCAGGTGCCGTTTTCCCAAGCTGGGTGATCCT
GAGGACCCTTGTCATCGCCAATTTCAAGTGCCGGCGACTGGATGGCATGGAGACGG
CGTTTCAAGAGGTGAAGGCCAGAGGCTACAACCCGGACCTCGTGATATTCAACTCC
ATGCTGTCCATCTACGCGAAGAACGGGATGTACAGCAAGGCCACCGAGGTCTTCGA
CTCCATCAAGCGGAGCGGGCTGAGCCCCGACCTCATCACCTACAACAGCCTGATGG
ACATGTACGCCAAGTGCAGCGAGTCGTGGGAGGCCGAGAAGATACTGAACCAGCTC
AAGTGCTCCCAGACGATGAAGCCCGACGTGGTGTCCTACAACACGGTCATAAACGG
GTTCTGCAAGCAGGGGCTGGTGAAAGAGGCCCAGAGGGTCCTCTCGGAGATGGTCG
CCGACGGCATGGCCCCCTGCGCCGTGACCTACCACACGCTCGTCGGGGGTTACTCCA
GCCTGGAGATGTTCAGCGAGGCCAGGGAGGTCATCGGCTACATGGTCCAGCACGGC
CTCAAGCCTATGGAGCTGACCTACAGGAGAGTCGTCGAGAGCTACTGCAGAGCGAA
GCGGTTCGAGGAGGCTCGCGGCTTCCTGTCCGAGGTCTCGGAGACCGACCTGGATTT
TGACAAGAAGGCGCTCGAAGCCTATATAGAGGATGCGCAGTTTGGAAGGTAG (SEQ
Phaseolin promoter
SEQ ID NO: 266
ggtcgacggtatcgataagcttgatatcgaattcctgcagcccaattcattgtactc
ccagtatcattatagtgaaagttttggctctctcgccggtggttttttacctctatt
taaaggggattccacctaaaaattctggtatcattctcacatacttgaacataattt
ctcataatcatggttgaaattatcacgcttccgcacacgatatccctacaaatttat
tatttgttaaacattttcaaaccgcataaaattttatgaagtcccgtctatctttaa
tgtagtctaacattttcatattgaaatatataatttacttaatatagcgaggtagaa
agcataatgatttattcttattcacttcatataaatgtttaatatacaatataaaca
aattctttaccttaagaaggatttcccattttatattttaaaaatatatttatcaaa
tatttttcaaccacgtaaatctcataataataagttgtttcaaaagtaataaaattt
aactccataatttttttattcgactgatcttaaagcaacacccagtgacacaactag
ccatttttttctttggataaaaaaatccaattatcattgtattttttttatacaatg
aaaatttcaccaaacaatcatttgtggtatttctgaagcaagtcatgttatgcaaaa
ttctataattcccatttgacactacggaagtaactgaagatctgcttttacatgcga
gacacatcttctaaagtaattttaataatagttactatattcaagatttcatatatc
aaatactcaatattacttctaaaaaattaattagatataattaaaatattacttttt
aattttaatttaattgttgaatttgtgactattgatttattattctactatgtttaa
attgttttatagatagtttaaagtaaatataagtaatgtagtagagtgttagagtgt
taccctaaaccataaactataacatttatggtggactaattttcatatatttcttat
tgcattaccttttcaggtatgtaagtccgtaactagaattactgtgggttgccatgg
catctgtggtcttttggttcatgcatggatgcttgcgcaagaaaaagacaaagaaca
aagaaaaaagacaaaacagagagacaaaacgcaatcacacaaccaactcaaattagt
cactggctgatcaagatcgccgcgtccatgtatgtctaaatgccatgcaaagcaaca
cgtgcttaacatgcactttaaatggctcacccatctcaacccacacacaaacacatt
gcctattcttcatcatcaccacaaccacctgatatattcattctatccgccacacaa
tttcttcacttcaacacacgtcaacctgca

The Arabidopsis nuclear genome encodes >400 Pentatricopeptide Repeat Proteins (PPRs), of which PPR10 is a member (Barkan and Small, 2014). Other P-type proteins that function similar to PPR10 are the Arabidopsis HCF152 and PGR3 proteins which is required for the accumulation of transcripts cleaved in the psbH-petB intergenic region and petL operon, respectively (Meierhoff et al., 2003; Yamazaki et al., 2004). Zea maize CRP1 is involved in the processing and translation of the chloroplast petD and petA RNAs (Fisk et al., 1999). HCF107, a member in the half-a-tetratricopeptide (HAT) family, also defines the processed end of psbH and enhance its translation by remodeling its 5′ UTR (Hammani et al., 2012). These proteins with their cognate binding site can be engineered to test and establish similar chloroplast transgene regulation system as PPR10.

Targeted Mutagenesis of Brassica napus ACC2 Genes to Obtain Spectinomycin Hypersensitive Plants

Chloroplast genome engineering in crops enables many applications, including improvement of photosynthetic efficiency, incorporation of novel metabolic pathways and delivery of vaccines in veterinary applications. This platform technology is absent in oilseed rape (Brassica napus or canola) due to its tolerance to spectinomycin, the selective agent used to obtain plants with transformed chloroplast genomes. We delete the ACC2 gene copies in the nuclear genome of oilseed rape to obtain spectinomycin hypersensitive. chloroplast transformation competent lines.

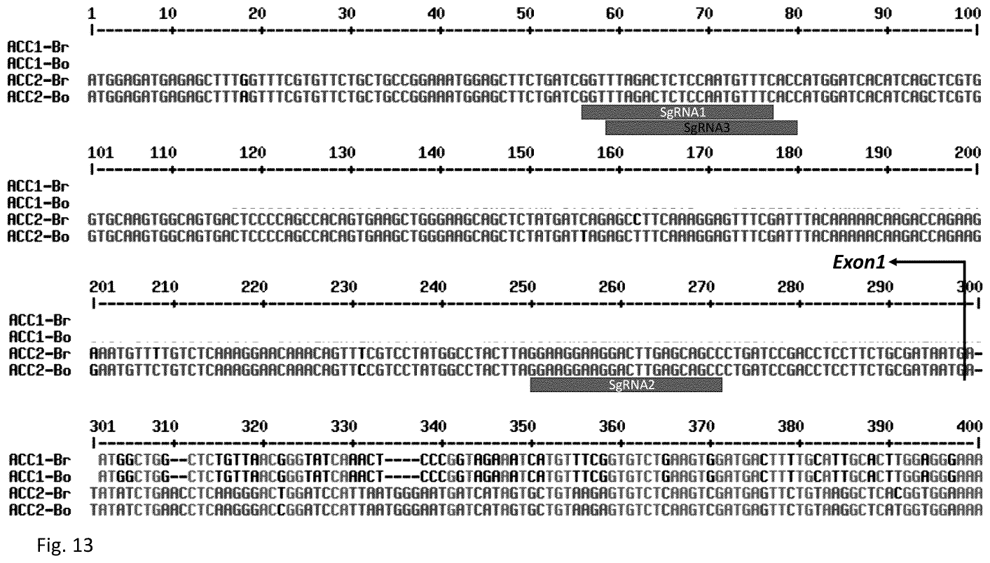

Brassica napus is a recent amphiploid hybrid of Brassica rapa and Brassica oleracea, and therefore carries at least one copy of each gene from the parental species. Because the common ancestor of the parental species underwent a genome triplication, this number may be as high as six. The B. napus cv Darmor-bzh darft genome available at the Genoscope website has only a single annotated ACC2 gene copy for each of the parental genomes: the Brassica rapa-like ACC2-Br BnaA06g04070D gene encoded in chromosome A6 and the Brassica oleracea-like ACC2-Bo BnaC06g01580D on chromosome C6. If multiple ACC2 gene copies are present, we hypothesized that over evolutionary time single nucleotide polymorphic mutations must have accumulated unique to each gene. To obtain information about the actual number of ACC2 gene copies and facilitate the design of gRNAs that simultaneously target each nuclear ACC2 gene copies, we cloned and sequenced PCR products of the N-terminal regions. Analyses of the data indicates that there are at least three B. rapa-like copies and two B. oleracea-like copies present in the B. napus cv. Westar nuclear genome. Inspection of the N-terminal extension lead to the identification of 28 potential sgRNAs with a GGN PAM sequence (Table 4). SgRNA3 was selected to target a single site and sgRNA1 and sgRNA2 to target two sites in the ACC2 N-terminal extension (FIG. 13). The benefit of targeting two sites is a deletion of DNA segment between the two sites, that may be used for tracking the mutant alleles by PCR.

TABLE 4

| | Genomic Sequence | | | |
|---|---|---|---|---|
| Target | (5′-3′) | Strand | Forward Oligo | Reverse Oligo |
| sgRNA1 | ggtttagactctccaatgtttc | + | GATTGctttgtaacctctcagatt | AAACaatctgagaggttacaaagC |
| sgRNA2 | ggaaggaaggacttgagcagcc | + | GATTGccgacgagttcaggaagga | AAACtccttcctgaactcgtcggC |
| sgRNA3 | ggtgaaacattggagagtctaa | − | GATTGaatctgagaggttacaaag | AAACctttgtaacctctcagattC |
| 4 | ggagcttctgatcggtttagac | + | | |
| 5 | ggtgcaagtggcagtgactccc | + | | |
| 6 | gacaccgacccctcagtgacgg | + | | |
| 7 | ggagtttcgatttacaaaaaca | + | | |
| 8 | ggcctacttaggaaggaaggac | + | | |
| 9 | ggaaggacttgagcagccctga | + | | |
| 10 | gtcctatggcctacttaggaagg | + | | |
| 11 | ggacttgagcagccctgatccg | + | | |
| 12 | atggcctacttaggaaggaagg | + | | |
| 13 | cgacctccttctgcgataatgg | + | | |
| 14 | ggagagtctaaaccgatcagaa | − | | |
| 15 | ggagtcactgccacttgcacca | − | | |
| 16 | ggggagtcactgccacttgcacc | − | | |
| 17 | ggctggggagtcactgccacttg | − | | |
| 18 | ggtcttgtttttgtaaatcgaa | − | | |
| 19 | tccttcctaagtaggccatagg | − | | |
| 20 | aagtccttccttcctaagtagg | − | | |
| 21 | ggctgctcaagtccttccttcc | − | | |
| 22 | gcagaaggaggtcggatcaggg | − | | |
| 23 | gggctgctcaagtccttccttc | − | | |
| 24 | cgcagaaggaggtcggatcagg | − | | |
| 25 | cattatcgcagaaggaggtcgg | − | | |
| 26 | ggtcggatcagggctgctcaag | − | | |
| 27 | ggaggtcggatcagggctgctc | − | | |
| 28 | agcaaaccattatcgcagaagg | − | | |

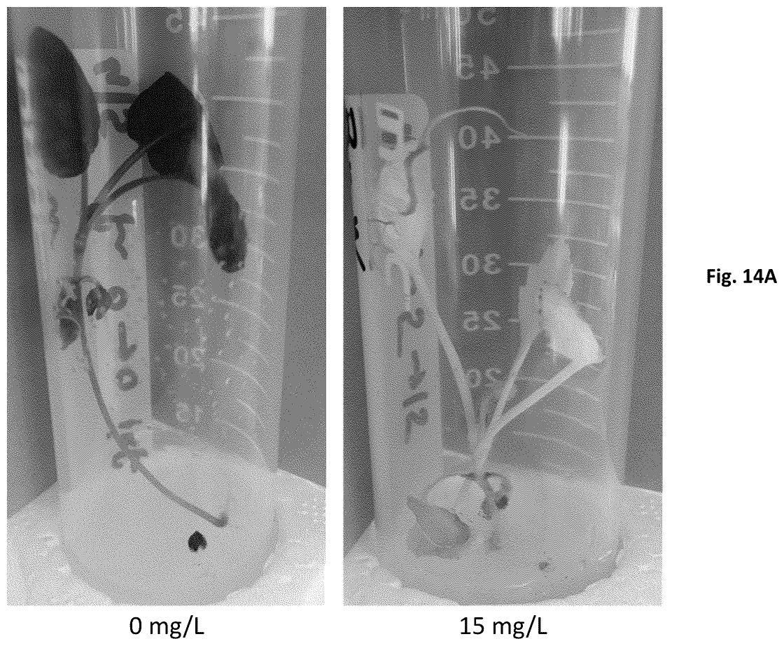

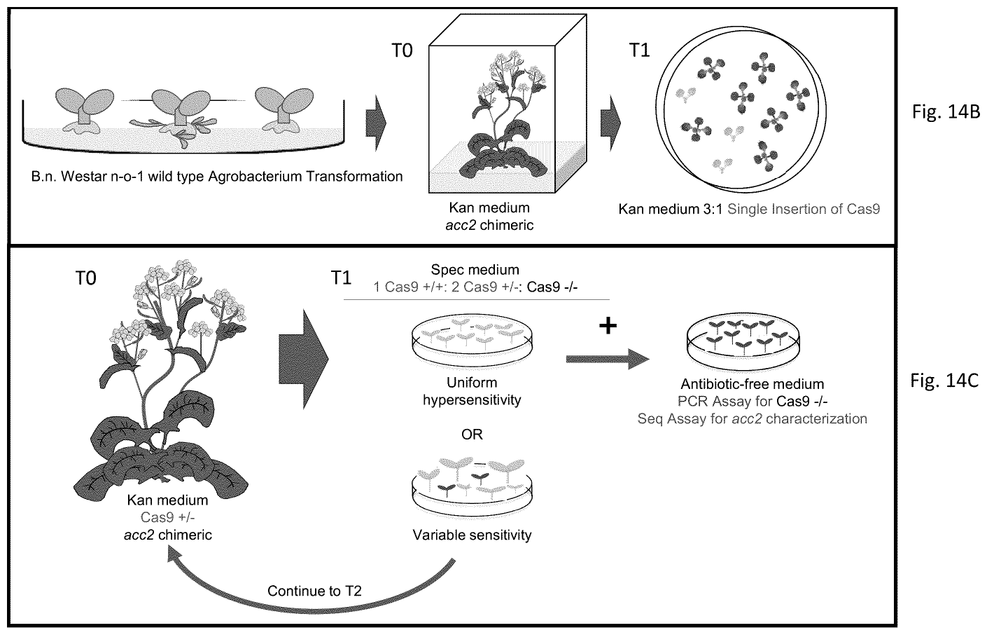

CRSPR/Cas9-mediated gene ACC2 gene editing in Brassica napus is carried out using the vector system developed in the Jiang-Kang Zhu laboratory (Mao et al., 2013; Liu et al., 2015). Single-stranded oligonucleotides were designed to fit the BbsI-digested p998/psgR-cas9-At vector, a pCAMBIA2300 vector derivative (Table 4). To accommodate the Arabidopsis U6 promoter, a G nucleotide was added at the end opposite to the PAM sequence. Agrobacterium vectors carrying two sgRNAs were obtained following the detailed protocol of Liu et al. (2015). Agrobacterium vectors carrying the sgRNAs were then introduced into Agrobacterium strain EHA105 or GV3101, and transformed into B. napus cotyledons following the protocol of Bates at all. (Bates et al., 2017). Progress in losing ACC2 activity is tracked by the absence of leaf formation on germinating seedlings. A tolerant B. napus seedling with well-developed leaves is shown in FIG. 14A. FIGS. 14B and 14C show a flowchart to obtain Cas9-free spectinomycin hypersensitive acc2 Brassica napus. (14B) Selection of CRISPR/Cas9 transgenic plants by kanamycin resistance. (14C) Hypersensitivity bioassay identifies T1 families with putative knockouts in all ACC2 copies. leading to the isolation of Cas9-free acc2 individuals. In certain instances, hypersensitivity will be uniform in the plant. Non-uniform hypersensitivity to spectinomycin will prompt an additional cycle of screening in the next seed generation.

### REFERENCES

Babiychuk E, Vandepoele K, Wissing J, Garcia-Diaz M, De Rycke R, Akbari H, Joubes J, Beeckman T, Jansch L, Frentzen M, Van Montagu MC, Kushnir S (2011) Plastid gene expression and plant development require a plastidic protein of the mitochondrial transcription termination factor family. Proc Natl Acad Sci USA 108: 6674-6679

Barkan A, Rojas M, Fujii S, Yap A, Chong YS, Bond CS, Small I (2012) A Combinatorial Amino Acid Code for RNA Recognition by Pentatricopeptide Repeat Proteins. PLOS Genetics 8

Barkan A, Small I (2014) Pentatricopeptide repeat proteins in plants. Annu Rev Plant Biol 65: 415-442

Belhaj K, Chaparro-Garcia A, Kamoun S, Nekrasov V (2013) Plant genome editing made easy: targeted mutagenesis in model and crop plants using the CRISPR/Cas system. Plant Methods 9: 39

Bock R (2015) Engineering Plastid Genomes: Methods. Tools, and Applications in Basic Research and Biotechnology. Annu Rev Plant Biol 66: 211-241

Brooks C, Nekrasov V, Lippman ZB, Van Eck J (2014) Efficient gene editing in tomato in the first generation using the clustered regularly interspaced short palindromic repeats/CRISPR-associated9 system. Plant Physiol 166: 1292-1297

Carrer H, Staub JM, Maliga P (1991) Gentamycin resistance in Nicotiana conferred by AAC(3)-I, a narrow substrate specificity acetyl transferase. Plant Mol Biol 17: 301-303

Carrillo N, Seyer P, Tyagi A, Herrmann RG (1986) Cytochrome b-559 genes from Oenothera hookeri and Nicotiana tabacum show a remarkably high degree of conservation as compared to spinach. The enigma of cytochrome b-559: highly conserved genes and proteins but no known function. Curr Genet 10: 619-624

Chakrabarti SK, Lutz KA, Lertwirijawong B, Svab Z, Maliga P (2006) Expression of the cry9Aa2 B.t. gene in the tobacco chlroplasts confers resistance to potato tuber moth. Transgenic Res 15: 481-488

Cheng L, Li HP, Qu B, Huang T, Tu JX, Fu TD, Liao YC (2010) Chloroplast transformation of rapeseed (Brassica napus) by particle bombardment of cotyledons. Plant Cell Rep 29: 371-381

Chupeau MC, Granier F, Pichon O, Renou JP, Gaudin V, Chupeau Y (2013) Characterization of the carly events leading to totipotency in an Arabidopsis protoplast liquid culture by temporal transcript profiling. Plant Cell 25: 2444-2463

Clough SJ, Bent AF (1998) Floral dip: a simplified method for Agrobacterium-mediated transformation of Arabidopsis thaliana. Plant J 16: 735-743

Corpet F (1988) Multiple sequence alignment with hierarchical clustering. Nucleic Acids Res 16: 10881-10890

De Jaegert G, Scheffer S, Jacobs A, Zambre M, Zobell O, Goossens A, Depicker A, Angenon G (2002) Boosting heterologous protein production in transgenic dicotyledonous seeds using Phaseolis vulgaris regulatory asequences. Nat Biotechnol 20: 1265-1268

Dufourmantel N, Dubald M, Matringe M, Canard H, Garcon F, Job C, Kay E, Wisniewski JP, Ferullo JM, Pelissier B, Sailland A, Tissot G (2007) Generation and characterization of soybean and marker-free tobacco plastid transformants over-expressing a bacterial 4-hydroxyphenylpyruvate dioxygenase which provides strong herbicide tolerance. Plant Biotechnol J 5: 118-133

Dufourmantel N, Pelissier B, Garcon F, Peltier G, Ferullo JM, Tissot G (2004) Generation of fertile transplastomic soybean. Plant Mol Biol 55: 479-489

Ellerstrom M, Stalberg K, Ezcurra I, Rask L (1996) Functional dissection of a napin gene promoter: Identification of promoter elements required for embryo and endosperm-specific transcription. Plant Mol Biol 32: 1019-1027

Feng Z, Mao Y, Xu N, Zhang B, Wei P, Yang DL, Wang Z, Zhang Z, Zheng R, Yang L, Zeng L, Liu X, Zhu JK (2014) Multigeneration analysis reveals the inheritance. specificity, and patterns of CRISPR/Cas-induced gene modifications in Arabidopsis. Proc Natl Acad Sci U S A 111: 4632-4637

Fisk DG, Walker MB, Barkan A (1999) Molecular cloning of the maize gene erpl reveals similarity between regulators of mitochondrial and chloroplast gene expression. EMBO J 18: 2621-2630

Gimpel JA, Nour-Eldin HH, Scranton MA, Li D, Mayfield SP (2016) Refactoring the Six-Gene Photosystem II Core in the Chloroplast of the Green Algae Chlamydomonas reinhardtii. ACS Synth Biol 5: 589-596

Hammani K, Cook WB, Barkan A (2012) RNA binding and RNA remodeling activities of the half-a-tetratricopeptide (HAT) protein HCF107 underlie its effects on gene expression. Proc Natl Acad Sci USA 109: 5651-5656

Hanson MR, Lin MT, Carmo-Silva AE, Parry MA (2016) Towards engineering carboxysomes into C3 plants. Plant J 87: 38-50

Hou BK, Zhou YH, Wan LH, Zhang ZL, Shen GF, Chen ZH, Hu ZM (2003) Chloroplast transformation in oilseed rape. Transgenic Res 12: 111-114

Howell EC, Kearsey M.J, Jones GH, King GJ, Armstrong S.J (2008) A and C genome distinction and chromosome identification in Brassica napus by sequential fluorescence in situ hybridization and genomic in situ hybridization. Genetics 180: 1849-1857

Kanamoto H, Yamashita A, Asao H, Okumura S, Takase H, Hattori M, Yokota A, Tomizawa K (2006) Efficient and stable transformation of Lactuca sativa L. cv. Cisco (lettuce) plastids. Transgenic Res 15: 205-217

Koornneef M, Meinke D (2010) The development of Arabidopsis as a model plant. Plant J 61: 909-921

Kuroda H, Maliga P (2001) Complementarity of the 16S rRNA penultimate stem with sequences downstream of the AUG destabilizes the plastid mRNAs. Nucleic Acids Res 29: 970-975

Kuroda H, Maliga P (2001) Sequences downstream of the translation initiation codon are important determinants of translation efficiency in chloroplasts. Plant Physiol 125: 430-436

Li JF, Zhang D, Sheen J (2014) Cas9-based genome editing in Arabidopsis and tobacco. Methods Enzymol 546: 459-472

Liu CW, Lin CC, Chen J.J, Tseng M.J (2007) Stable chloroplast transformation in cabbage (Brassica oleracea L. var. capitata L.) by particle bombardment. Plant Cell Rep 26: 1733-1744

Liu CW, Lin CC, Yiu JC, Chen JJ, Tseng M.J (2008) Expression of a Bacillus thuringiensis toxin (crylAb) gene in cabbage (Brassica oleracea L. var. capitata L.) chloroplasts confers high insecticidal efficacy against Plutella xylostella. Theor Appl Genet 117: 75-88

Liu F, Cao M, Yao L, Li Y, Robaglia C, Tourneur C (1998) In planta transformation of pakchoi (Brassica campestris l. ssp. chinensis) by infiltration of adult plants with Agrobacterium. Acta Horticult 467: 187

Liu X, Brost J, Hutcheon C, Guilfoil R, Wilson AK, Leung S, Shewmaker CK, Rooke S, Nguyen T, Kiser J, De Rocher J (2012) Transformation of the oilseed crop Camelina sativa by Agrobacterium-mediated floral dip and simple large-scale screening of transformants. In Vitro Cellular & Developmental Biology—Plant 48: 462-468

Lu CF, Kang JL (2008) Generation of transgenic plants of a potential oilseed crop Camelina sativa by Agrobacterium-mediated transformation. Plant Cell Rep 27: 273-278

Lutz KA, Azhagiri A, Maliga P (2011) Transplastomics in Arabidopsis: Progress towards developing an efficient method. In RP Jarvis, ed. Chloroplast research in Arabidopsis, Vol 774. Springer Science+Business Media, LLC, New York, pp 133-147.

Lutz KA, Knapp JE, Maliga P (2001) Expression of bar in the plastid genome confers herbicide resistance. Plant Physiol 125: 1585-1590

Lutz KA, Martin C, Khairzada S, Maliga P (2015) Steroid-inducible BABY BOOM system for development of fertile Arabidopsis thaliana plants after prolonged tissue culture. Plant Cell Rep 34: 1849-1856

Mali P, Esvelt KM, Church GM (2013) Cas9 as a versatile tool for engineering biology. Nat Methods 10: 957-963

Maliga P (2012) Plastid transformation in flowering plants. In R Bock. V Knoop, eds, Genomics of Chloroplasts and Mitochondria. Vol 35. Springer. pp 393-414

Maliga P, Bock R (2011) Plastid biotechnology: food, fuel and medicine for the 21st century. Plant Physiol 155: 1501-1510

Maliga P, Tungsuchat-Huang T (2014) Plastid transformation in Nicotiana tabacum and Nicotiana sylvestris by biolistic DNA delivery to leaves. In P Maliga, ed. Chloroplast Biotechnology: Methods and Protocols, Vol 1132. Springer Science+Business Media, New York, pp 147-163

Mao Y, Zhang H, Xu N, Zhang B, Gou F, Zhu JK (2013) Application of the CRISPR-Cas system for efficient genome engineering in plants. Mol Plant 6: 2008-2011

Marton L, Browse J (1991) Facile transformation of Arabidopsis. Plant Cell Rep 10: 235-239

Meierhoff K, Felder S, Nakamura T, Bechtold N, Schuster G (2003) HCF152, an Arabidopsis RNA binding pentatricopeptide repeat protein involved in the processing of chloroplast psbB-psbT-psbH-petB-petD RNAs. Plant Cell 15: 1480-1495

Meng BY, Tanaka M, Wakasugi T, Ohme M, Shinozaki K, Sugiura M (1988) Cotranscription of the genes encoding two P700 chlorophyll a apoproteins with the gene for ribosomal protein CS14: determination of the transcriptional initiation site by in vitro capping. Curr Genet 14: 395-400

Motte H, Galuszka P, Spichal L, Tarkowski P, Plihal O, Smehilova M, Jaworek P, Vereecke D, Werbrouck S, Geelen D (2013) Phenyl-adenine, identified in a LIGHT-DEPENDENT SHORT HYPOCOTYLS4-assisted chemical screen, is a potent compound for shoot regeneration through the inhibition of CYTOKININ OXIDASE/DEHYDROGENASE activity. Plant Physiol 161: 1229-1241

Nugent GD, Coyne S, Nguyen TT, Kavanagh TA, Dix P.J (2006) Nuclear and plastid transformation of Brassica oleracea var. botrytis (cauliflower) using PEG-mediated uptake into protoplasts. Plant Sci 170: 135-142

Parker N, Wang Y, Meinke D (2014) Natural variation in sensitivity to a loss of chloroplast translation in Arabidopsis. Plant Physiol 166: 2013-2027

Parker N, Wang Y, Meinke D (2016) Analysis of Arabidopsis Accessions Hypersensitive to a Loss of Chloroplast Translation. Plant Physiol 172: 1862-1875

Qing CM, Fan L, Lei Y, Bouchez D, Tourneur C, Yan L, Robaglia C (2000) Transformation of Pakchoi (Brassica rapa L. ssp chinensis) by Agrobacterium infiltration. Molecular Breeding 6: 67-72

Ruf S, Hermann M, Berger IJ, Carrer H, Bock R (2001) Stable genetic transformation of tomato plastids: foreign protein expression in fruit. Nat Biotechnol 19: 870-875

Ruhlman T, Verma D, Samson N, Daniell H (2010) The role of heterologous chloroplast sequence elements in transgene integration and expression. Plant Physiol 152: 2088-2104

Scharff LB, Bock R (2014) Synthetic biology in plastids. Plant J 78: 783-798

Schneider A, Stelljes C, Adams C, Kirchner S, Burkhard G, Jarzombski S, Broer I, Horn P, Elsayed A, Hagl P, Leister D, Koop HU (2015) Low frequency paternal transmission of plastid genes in Brassicaceae. Transgenic Res 24: 267-277

Schottkowski M, Peters M, Zhan Y, Rifai O, Zhang Y, Zerges W (2012) Biogenic membranes of the chloroplast in Chlamydomonas reinhardtii. Proc Natl Acad Sci USA 109: 19286-19291

Schulte W, Topfer R, Stracke R, Schell J, Martini N (1997) Multi-functional acetyl-CoA carboxylase from Brassica napus is encoded by a multi-gene family: indication for plastidic localization of at least one isoform. Proc Natl Acad Sci U S A 94: 3465-3470

Shao M, Blechl A, Thomson JG (2017) Small serine recombination systems ParA-MRS and CinH-RS2 perform precise excision of plastid DNA. Plant Biotechnol J 15: 1577-1589

Shao M, Kumar S, Thomson JG (2014) Precise excision of plastid DNA by the large serine recombinase Bxb1. Plant Biotechnol J 12: 322-329

Sharwood RE, Ghannoum O, Whitney SM (2016) Prospects for improving CO2 fixation in C3-crops through understanding C4-Rubisco biogenesis and catalytic diversity. Curr Opin Plant Biol 31: 135-142

Shimizu M, Goto M, Hanai M, Shimizu T, Izawa N, Kanamoto H, Tomizawa K, Yokota A, Kobayashi H (2008) Selectable tolerance to herbicides by mutated acetolactate synthase genes integrated into the chloroplast genome of tobacco. Plant Physiol 147: 1976-1983

Sikdar SR, Serino G, Chaudhuri S, Maliga P (1998) Plastid transformation in Arabidopsis thaliana. Plant Cell Rep 18: 20-24

Sinagawa-Garcia SR, Tungsuchat-Huang T, Paredes-Lopez O, Maliga P (2009) Next generation synthetic vectors for transformation of the plastid genome of higher plants. Plant Mol Biol 70: 487-498

Skarjinskaia M, Svab Z, Maliga P (2003) Plastid transformation in Lesquerella fendleri, an oilseed Brassicacea. Transgenic Res 12: 115-122

Sohrabi M, Zebarjadi A, Najaphy A, Kahrizi D (2015) Isolation and sequence analysis of napin seed specific promoter from Iranian Rapeseed (Brassica napus L.). Gene 563: 160-164

Song K, Osborn TC (1992) Polyphyletic Origins of Brassica-Napus—New Evidence Based on Organelle and Nuclear Rflp Analyses. Genome 35: 992-1001

Staub JM, Maliga P (1995) Expression of a chimeric uidA gene indicates that polycistronic mRNAs are efficiently translated in tobacco plastids. Plant J 7: 845-848

Stitt M, Lunn J, Usadel B (2010) Arabidopsis and primary photosynthetic metabolism—more than the icing on the cake. Plant J 61: 1067-1091

Svab Z, Hajdukiewicz P, Maliga P (1990) Stable transformation of plastids in higher plants. Proc Natl Acad Sci USA 87: 8526-8530

Svab Z, Maliga P (1993) High-frequency plastid transformation in tobacco by selection for a chimeric aadA gene. Proc Natl Acad Sci USA 90: 913-917

Tan HL, Yang XH, Zhang FX, Zheng X, Qu CM, Mu JY, Fu FY, Li JA, Guan RZ, Zhang HS, Wang GD, Zuo JR (2011) Enhanced Seed Oil Production in Canola by Conditional Expression of Brassica napus LEAFY COTYLEDONI and LECI-LIKE in Developing Seeds. Plant Physiol 156: 1577-1588

Tungsuchat-Huang T, Maliga P (2012) Visual marker and Agrobacterium-delivered recombinase enable the manipulation of the plastid genome in greenhouse-grown tobacco plants. Plant J 70: 717-725

Valkov VT, Gargano D, Manna C, Formisano G, Dix P.J, Gray JC, Scotti N, Cardi T (2011) High efficiency plastid transformation in potato and regulation of transgene expression in leaves and tubers by alternative 5′ and 3′ regulatory sequences. Transgenic Res 20: 137-151

Verma S, Chinnusamy V, Bansal K (2008) A Simplified Floral Dip Method for Transformation of Brassica napus and B. carinata. J Plant Biochemistry and Biotechnology 17: 197-200

Wallis JG, Browse J (2010) Lipid biochemists salute the genome. Plant J 61: 1092-1106

Wang TW, Wu W, Zhang CG, Nowack LM, Liu ZD, Thompson JE (2005) Antisense suppression of deoxyhypusine synthase by vacuum-infiltration of Agrobacterium enhances growth and seed yield of canola. Physiol Plant 124: 493-503

Wang WC, Menon G, Hansen G (2003) Development of a novel Agrobacterium-mediated transformation method to recover transgenic Brassica napus plants. Plant Cell Rep 22: 274-281

Weigel D, Mott R (2009) The 1001 genomes project for Arabidopsis thaliana. Genome biology 10: 107

Willey DL, Gray JC (1989) Two small open reading frames are co-transcribed with the pea chloroplast genes for the polypeptides of cytochrome b-559. Curr Genet 15: 213-220

Willey DL, Gray JC (1990) An open reading frame encoding a putative haem-binding polypeptide is cotranscribed with the pea chloroplast gene for apocytochrome f. Plant Mol Biol 15: 347-356

Wilson DN (2014) Ribosome-targeting antibiotics and mechanisms of bacterial resistance. Nat Rev Microbiol 12: 35-48

Wirmer J, Westhof E (2006) Molecular contacts between antibiotics and the 30S ribosomal particle. Methods Enzymol 415: 180-202

Xie K, Yang Y (2013) RNA-guided genome editing in plants using a CRISPR-Cas system. Mol Plant 6: 1975-1983

Yamazaki H, Tasaka M, Shikanai T (2004) PPR motifs of the nucleus-encoded factor, PGR3, function in the selective and distinct steps of chloroplast gene expression in Arabidopsis. Plant J 38: 152-163

Yang L, Guell M, Niu D, George H, Lesha E, Grishin D, Aach J, Shrock E, Xu W, Poci J, Cortazio R, Wilkinson RA, Fishman JA, Church G (2015) Genome-wide inactivation of porcine endogenous retroviruses (PERVs). Science 350: 1101-1104

Ye GN, Colburn S, Xu CW, Hajdukiewicz PTJ, Staub JM (2003) Persistance of unselected transgenic DNA during a plastid transformation and segregation approach to herbicide resistance. Plant Physiol 133: 402-410

Zhang J, Khan SA, Hasse C, Ruf S, Heckel DG, Bock R (2015) Pest control. Full crop protection from an insect pest by expression of long double-stranded RNAs in plastids. Science 347: 991-994

Zhao X, Liang G, Li X, Zhang X (2014) Hormones regulate in vitro organ regeneration from leaf-derived explants in Arabidopsis. Am J Plant Sci 5: 3535-3550

Zhao XY, Su YH, Zhang CL, Wang L, Li X, Zhang XS (2013) Differences in capacities of in vitro organ regeneration between two Arabidopsis ecotypes Wassilewskija and Columbia. Plant Cell Tissue Organ Cult. 112: 65-74

Zubko MK, Day A (1998) Stable albinism induced without mutagenesis: a model for ribosome-free plastid inheritance. Plant J 15: 265-271

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for increasing plastid transformation efficiency in plastids of a Brassica ssp. plant, comprising;
a) providing a plant comprising a nonfunctional or defective ACC2 nuclear gene;
b) introducing one or more plastid transformation vectors into the plastids in cells from said plant, said one or more vectors comprising an aadA spectinomycin resistance marker sequence and a nucleic acid sequence encoding a protein of interest;
c) contacting said cells with spectinomycin and selecting plant cells which are resistant to spectinomycin and accumulate said protein of interest in said plastids; and
d) culturing said plant cells under conditions suitable to regenerate a transplastomic plant therefrom.

2. (canceled)

3. The method of claim 1, wherein said protein of interest is green fluorescent protein.

4. The method of claim 1, wherein the plant of step a) is a naturally occurring mutant which encodes non-functional or defective ACC2.

5. The method of claim 1, wherein said ACC2 gene is inactivated in said plant using CRISPR/Cas prior to plastid transformation.

6.-7. (canceled)

8. The method of claim 1, further comprising excising said aadA spectinomycin resistance marker sequence from said plant.

9. The method of claim 5, wherein said protein of interest is selected from the group consisting of a protein conferring herbicide resistance, a protein conferring insect resistance, a vaccine, an antibody, regulatory RNA, dsRNA, siRNA, shRNA and insecticidal proteins.

10. A method for seed-specific plastid expression comprising:
a) introducing a nuclear expression vector encoding a modified PPR10 binding protein driven by a seed-specific promoter and
b) a plastid expression vector encoding a gene of interest linked to an upstream PPR10 binding site, wherein nuclear-expressed PPR10 is imported into plastids and binds said PPR 10 binding site to drive expression of the gene of interest in seed plastids.

11. The method of claim 10, wherein said vector comprises a seed specific promoter selected from a napin or a phaseolin gene promoter.

12. The method of claim 10, wherein said modified PPR10 binding protein is PPR10^{GG} encoded by SEQ ID NO: 265.

13. The method of claim 10, wherein said PPR10 binding site encoded by SEQ ID NO: 261.

14. The method of claim 10, further comprising plastid expression of an aadA spectinomycin resistance gene.

15. The method of claim 10, wherein the plastid expressed gene of interest is linked to an upstream sequence encoding a maize atpH gene and/or tRNA sequence in said plastid vector.

16. A method for increasing plastid transformation efficiency in plastids of a Brassica ssp. plant recalcitrant to plastid transformation, comprising;
a) providing a plant comprising a nonfunctional ACC2 nuclear gene;
b) introducing one or more plastid transformation vectors into the plastids in cells from said plant, said one or more vectors comprising a nucleic acid sequence conferring resistance to said plastid translation inhibitor, and a nucleic acid sequence encoding a protein of interest;
c) contacting said cells with said inhibitor and selecting plant cells which are resistant to said inhibitor and accumulate said protein of interest in said plastids; and
d) culturing said plant cells under conditions suitable to regenerate a transplastomic plant therefrom.

17. The method of claim 16, wherein said plastid translation inhibitor is selected from the group consisting of kanamycin, chloramphenicol, tobramycin and gentamycin.

18. The method of claim 17, wherein inhibitor is kanamycin.

19. The method of claim 17, wherein said inhibitor is chloramphenicol and said nucleic acid encodes chloramphenicol acetyl transferase.

20. The method of claim 17, wherein said inhibitor is tobramycin.

21. The method of claim 17, wherein said inhibitor is gentamycin.
